SEQUENCE LISTING (19 sequences)

SEQ ID NO: 1 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 2 - protein, 30 aa, Homo sapiens
TRCPDGQFCPVACCLDPGGASYSCCRPLLD

SEQ ID NO: 3 - protein, 56 aa, Homo sapiens
GGPCQVDAHCSAGHSCIFTVSGTSSCCPFPEAVACGDGHHCCPRGFHCSADGRSCF

SEQ ID NO: 4 - protein, 57 aa, Homo sapiens
AIQCPDSQFECPDFSTCCVMVDGSWGCCPMPQASCCEDRVHCCPHGAFCDLVHTRCI

SEQ ID NO: 5 - protein, 56 aa, Homo sapiens
VMCPDARSRCPDGSTCCELPSGKYGCCPMPNATCCSDHLHCCPQDTVCDLIQSKCL

SEQ ID NO: 6 - protein, 56 aa, Homo sapiens
DVKCDMEVSCPDGYTCCRLQSGAWGCCPFTQAVCCEDHIHCCPAGFTCDTQKGTCE

SEQ ID NO: 7 - protein, 54 aa, Homo sapiens
VPCDNVSSCPSSDTCCQLTSGEWGCCPIPEAVCCSDHQHCCPQGYTCVAEGQCQ

SEQ ID NO: 8 - protein, 55 aa, Homo sapiens
IGCDQHTSCPVGQTCCPSLGGSWACCQLPHAVCCEDRQHCCPAGYTCNVKARSCE

SEQ ID NO: 9 - protein, 56 aa, Homo sapiens
DVECGEGHFCHDNQTCCRDNRQGWACCPYRQGVCCADRRHCCPAGFRCAARGTKCL

SEQ ID NO: 10 - DNA, 1782 bp, Homo sapiens
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 11 - protein, 317 aa, Artificial Sequence [sequence omitted]

SEQ ID NO: 12 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 13 - protein, 85 aa, Artificial Sequence
  Synthetic Construct
GGGGAGGGGAGGGGAGGGGAGGGPSLCGGELVDTLQFVCGDRGFYFSRPASRVSARSRGIVEECCFRSCDLALLETYCATPAKSE

SEQ ID NO: 14 - protein, 91 aa, Artificial Sequence
  Synthetic Construct
GAPGGGSPAPAPTPAPAPTPAPAGGGPSGAPLCGGELVDTLQFVCGDRGFYFSRPASRVSARSRGIVEECCFRSCDLALLETYCATPAKSE

SEQ ID NO: 15 - protein, 116 aa, Artificial Sequence
  Synthetic Construct
GAPGGSPAGSPTSTEEGTSESATPESGPGTSTEPSEGSAPGSPAGSPTSTGPSGAPLCGGELVDTLQFVCGDRGFYFSRPASRVSARSRGIVEECCFRSCDLALLETYCATPAKSE

SEQ ID NO: 16 - DNA, 260 bp, Artificial Sequence
  Synthetic Construct
ggcggaggcggagctggtggcggcggagcaggcggtggtggtgcaggcggcggaggtgctggcggaggaccatctctttgtggcggagaactggtggacaccctgcagttcgtgtgtggcgacagaggcttctactttagcagacccgccagcagagtgtccgccagatctagaggaatcgtggaagagtgctgcttcagaagctgcgacctggcactgctggaaacctactgtgccacaccagccaagagcgagtgatg

SEQ ID NO: 17 - DNA, 279 bp, Artificial Sequence
  Synthetic Construct
ggagcaccaggcggaggatctccagctcctgctcctacaccagctccagcaccgacgcctgctccagctggcggaggaccttctggtgcacctctttgtggcggagagctggtggataccctgcagttcgtgtgtggcgaccggggcttctactttagcagacctgccagcagagtgtccgccagatctagaggcatcgtggaagagtgctgcttcagaagctgcgacctggcactgctggaaacctactgtgccacaccagccaagagcgagtgatga

SEQ ID NO: 18 - DNA, 354 bp, Artificial Sequence
  Synthetic Construct
ggagcaccaggcggatctccagcaggatctccaacctctaccgaggaaggcacaagcgagtctgccacacctgagtctggacctggcacaagcacagagcctagcgaaggatctgccccaggttctcctgccggctctcctacaagtacaggaccttctggcgctccactgtgtggcggagaactggtggataccctgcagttcgtgtgcggcgacagaggcttctactttagcagacccgccagcagagtgtccgccagatctagaggaatcgtggaagagtgctgcttcagaagctgcgatctggcactgctggaaacctactgtgccacaccagccaagagcgagtgatga

SEQ ID NO: 19 - DNA, 1785 bp, Artificial Sequence
  Synthetic Construct
atgtggactctggtctcatgggtcgctctgacagcaggactggtcgcaggaacaagatgccccgatggacagttttgccccgtcgcttgctgtctggacccaggaggagcaagctactcctgctgtaggccactgctggataagtggcccaccacactgtcccgccacctgggaggaccatgccaggtggacgcacactgttccgccggacactcttgcatcttcacagtgtctggcaccagctcctgctgtccatttcctgaggcagtggcatgcggcgacggacaccactgctgtcccaggggcttccactgtagcgccgatggccggtcctgctttcagagaagcggcaacaattccgtgggcgccatccagtgtcctgacagccagttcgagtgcccagatttttccacctgctgcgtgatggtggatggctcttggggctgctgtccaatgccccaggccagctgctgtgaggacagggtgcactgctgtcctcacggcgccttctgtgatctggtgcacacacgctgcatcacccccacaggcacccaccctctggccaagaagctgccagcacagaggaccaacagggcagtggccctgtctagcagcgtgatgtgccccgacgcccggtctagatgccctgatggcagcacctgctgtgagctgccaagcggcaagtacggctgctgtcctatgccaaacgccacatgctgttccgaccacctgcactgctgtcctcaggacaccgtgtgcgatctgatccagtctaagtgcctgagcaaggagaatgccaccacagacctgctgacaaagctgcctgcccacaccgtgggcgacgtgaagtgtgatatggaggtgtcctgcccagatggctatacatgctgtcggctgcagtctggagcatggggatgctgtcccttcacccaggccgtgtgctgtgaggaccacatccactgctgtcctgccggctttacatgcgatacccagaagggcacatgcgagcagggccctcaccaggtgccatggatggagaaggcaccagcacacctgtccctgcccgaccctcaggccctgaagagagacgtgccttgtgataacgtgtctagctgcccatcctctgatacatgctgtcagctgacctctggcgagtggggctgctgtccaatccccgaggccgtgtgctgtagcgaccaccagcactgctgtcctcagggctatacctgcgtggcagagggacagtgccagaggggctccgagatcgtggcaggactggagaagatgccagcaaggagagcatctctgagccaccccagagacatcggctgtgatcagcacacaagctgcccagtgggacagacctgctgtccatccctgggaggctcttgggcatgctgtcagctgcctcacgccgtgtgctgtgaggatcggcagcactgctgtccagccggctacacatgcaatgtgaaggccagatcctgcgagaaggaggtggtgtctgcccagccagccaccttcctggcaaggagccctcacgtgggagtgaaggacgtggagtgtggcgagggccacttttgccacgacaaccagacatgctgtcgggataatagacagggctgggcctgctgtccatataggcagggcgtgtgctgtgcagataggcgccactgctgtccagcaggctttaggtgcgcagcaaggggaaccaagtgcctgagaagagaagccccccggtgggacgcccccctgagagaccctgccctgagacagctgctgtgatga

(19) United States
(12) Patent Application Publication
(10) Pub. No.: US 2022/0111005 A1
(43) Pub. Date: Apr. 14, 2022

(54) COMPOSITIONS AND METHODS FOR TREATING NEUROCOGNITIVE DISORDERS

(71) Applicant: AvroBio, Inc., Cambridge, MA (US)

(72) Inventors: Chris MASON, Cambridge, MA (US); Oliver COOPER, Jamaica Plain, MA (US); Mark DEANDRADE, Boston, MA (US); Robert PLASSCHAERT, Cambridge, MA (US); Nico Peter (Niek) VAN TIL, Leiden (NL)

(73) Assignees: AvroBio, Inc., Cambridge, MA (US); AvroBio, Inc., Cambridge, MA (US)

(21) Appl. No.: 17/427,298

(22) PCT Filed: Jan. 31, 2020
(86) PCT No.: PCT/US2020/016192

§ 371 Date: Jul. 30, 2021

Related U.S. Application Data
(60) Provisional application No. 62/800,096, filed on Feb. 1, 2019.

(51) Int. Cl. A61K 38/18; A61K 38/17; A61K 35/545; A61K 35/28; A61K 35/15; A61K 31/10; A61K 31/444; A61K 31/663; A61K 48/00; A61P 25/28; C12N 15/86; C12N 15/62

(52) U.S. Cl. CPC A61K 38/18; C12N 2830/008; A61K 35/545; A61K 35/28; A61K 35/15; A61K 31/10; A61K 31/444; A61K 31/663; A61K 48/0066; A61P 25/28; C12N 15/86; C12N 15/625; C12N 2730/10143; C12N 2740/16043; C12N 2740/15043; C12N 2750/14143; C12N 2840/445; A61K 38/1709

(57) ABSTRACT

Described herein are methods for treating a subject having or at risk of developing a neurocognitive disorder, such as frontotemporal lobar degeneration or neuronal ceroid lipofuscinosis, by administering cells that contain a transgene encoding a progranulin (PGRN) or a granulin (GRN) or cells that express the PGRN or the GRN to the subject. Also disclosed are compositions comprising cells containing the transgene encoding the PGRN or the GRN.

### SEQUENCE LISTING

The instant application contains a Sequence Listing which has been submitted electronically in ASCII format and is hereby incorporated by reference in its entirety. Said ASCII copy, created on Jan. 29, 2020 is named “51182-019WO2_Sequence_Listing_1.29.20_ST25” and is 22,275 bytes in size.

### FIELD OF THE INVENTION

The disclosure relates to compositions and methods for treating neurocognitive disorders, such as frontotemporal lobar degeneration and neuronal ceroid lipofuscinosis.

### BACKGROUND

Neurodegeneration is a pathophysiological process that is observed in a number of diseases associated with progressive dementia, such as frontotemporal lobar degeneration and neuronal ceroid lipofuscinosis. A key feature of this process is the neuronal degeneration and death that causes the wholesale destruction of brain tissue and the accompanying gamut of behavioral deficits including cognitive decline, language impairments, among others.

Frontotemporal lobar degeneration (FTLD) is a neurodegenerative disorder characterized by a complex clinical presentation that may include deficits in speech comprehension and production, poor motor planning and coordination, and/or loss of executive function characterized by lack of impulse control and a preference for perseverative behaviors. Clinical descriptions of FTLD separate into three distinct variants: 1) behavioral-frontotemporal dementia, characterized by profound changes in behavior, personality, and pronounced degeneration of the frontal lobe; 2) semantic dementia, characterized by insidious degradation of language and pronounced degeneration in the anterior temporal lobe; and 3) progressive nonfluent aphasia, characterized by deficits in speech and grammar and corresponding to degeneration of left perisylvian cortex. Histological analyses of post-mortem brain tissue from FTLD patients exhibit complex and heterogeneous neuropathological profiles with the common presentation of degeneration of neural tissue in the frontal and temporal lobes of the brain.

Neuronal ceroid lipofuscinosis (NCL) is an umbrella term for a clinically recognized collection of at least eight lysosomal storage disorders that are caused by the accumulations of lipofuscin within cells of the body, such as neuronal, liver, spleen, myocardium, and kidney cells. Lipofuscin is a lipopigment composed of fats and proteins. Patients having NCL exhibit profound neurodegeneration and progressive and irreversible loss of motor and cognitive abilities, although the disease severity and clinical presentation may depend on the particular NCL variant. Known variants of NCL include the infantile variant, also known as Santovuori-Haltia disease (SHD), the late infantile variant known as Jansky-Bielschowsky disease (JBD), the Finnish late infantile variant (FLI), the variant late infantile (VLI), the CLN7 variant (CLN7), the CLN8 variant (CLN8), the Turkish late infantile variant (TLI), the type 9 variant (T9), the CLN10 variant (CLN10), the CLN11 variant (CLN11), the juvenile variant also known as Batten disease (BD), and the adult variant also known as Kuf's disease (KD). SHD is associated with early visual loss that progressively turns to complete retinal blindness by the age of 2, followed by a vegetative state at 3 years, and brain death by year 4. This variant is also associated with the spontaneous occurrence of epileptic seizures. The JBD variant emerges between ages 2 to 4 and is associated with ataxia, epileptic seizures, progressive cognitive decline, and abnormal speech development and typically results in death by age 8. BD typically emerges between 4 and 10 years of age and include symptoms such as vision loss, epileptic seizures, cognitive dysfunction, and premature death. NCL patients having the KD variant generally present with milder symptoms than SHD and BD variants and have a life expectancy of around 40 years.

Existing treatments for FTLD and NCL strive to ameliorate disease symptomology, but therapies targeting the underlying neurodegeneration are lacking, thus underscoring the need for new therapeutic avenues.

### SUMMARY OF THE INVENTION

The present disclosure provides methods for treating a neurocognitive disorder (NCD), such as frontotemporal lobar degeneration (FTLD) or neuronal ceroid lipofuscinosis (NCL), by administering cells, such as pluripotent cells (e.g., embryonic stem cells (ESCs) or induced pluripotent stem cells (ISPCs)), multipotent cells (e.g., CD34+ cells such as, e.g., hematopoietic stem cells (HSCs) or myeloid precursor cells (MPCs)), blood lineage progenitor cells (BLPCS; e.g., monocytes), macrophages, microglial progenitor cells, or microglia containing a transgene encoding a progranulin (PGRN) or a granulin (GRN). The cells may be administered to a subject having an NCD by one or more of a variety of routes, including directly to the central nervous system of the subject (e.g., by intracerebroventricular injection) or systemically (e.g., by intravenous administration), among others. The disclosure also features compositions containing such cells, as well as kits containing these cells for the treatment of an NCD.

In a first aspect, the disclosure provides a method of treating a subject diagnosed as having an NCD (e.g., FTLD or NCL) by administering to the subject a composition containing a population of cells containing a transgene encoding a PGRN or a GRN. In some embodiments, the transgene encoding the PGRN or the GRN is capable of expression in a macrophage or a microglial cell. In some embodiments, the NCD is a major NCD. In some embodiments, the major NCD interferes with the subject's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the subject on a cognitive test that is at least two standard deviations away from the mean score of a reference population. In some embodiments, the NCD is a mild NCD. In some embodiments, the mild NCD does not interfere with the subject's independence and/or normal daily functioning. In some embodiments, the mild NCD is associated with a score obtained by the subject on a cognitive test that is between one to two standard deviations away from the mean score of a reference population. In some embodiments, the cognitive test is selected from the group consisting of Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE). In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the subject's age, medical history, education, socioeconomic status, and lifestyle. In some embodiments, the NCD is a frontotemporal NCD. In some embodiments the frontotemporal NCD is FTLD. In some embodiments, the NCD is due to a lysosomal disease. In some embodiments, the lysosomal disease is NCL.

In some embodiments, the PGRN is full-length PGRN, such as PGRN having an amino acid sequence of SEQ ID NO. 1, or a variant thereof having at least 85% sequence identity thereto (e.g., having at least 85%, 86%, 87%, 88%, 89%, 90%, 91%, 92%, 93%, 94%, 95%, 96%, 97%, 98%, 99%, or more sequence identity to SEQ ID NO. 1). In some embodiments, the PGRN comprises at least 2 (e.g., at least 2, 3, 4, 5, 6, 7, 8 or more) GRN domains having the amino acid sequence of any one of SEQ ID NOs. 2-9 or a variant thereof having at least 85% sequence identity thereto (e.g., at least 85%, 86%, 87%, 88%, 89%, 90%, 91%, 92%, 93%, 94%, 95%, 96%, 97%, 98%, 99%, or more sequence identity to any one of SEQ ID NOs. 2-9). In some embodiments, the PGRN comprises at least 2 (e.g., 2, 3, 4, 5, 6, 7, 8 or more) GRN domains. In some embodiments, the PGRN comprises at least 3 (e.g., at least 3, 4, 5, 6, 7, 8, or more) GRN domains. In some embodiments, the PGRN comprises at least 4 (e.g., at least 4, 5, 6, 7, 8 or more) GRN domains. In some embodiments, the PGRN comprises at least 5 (e.g., at least 5, 6, 7, 8 or more) GRN domains. In some embodiments, the PGRN comprises at least 6 (e.g., at least 6, 7, 8 or more) GRN domains. In some embodiments, the PGRN comprises at least 7 (e.g., at least 7, 8 or more) GRN domains. In some embodiments, the PGRN comprises at least 8 (e.g., at least 8 or more) GRN domains. In some embodiments, the PGRN comprises from 2 to 16 (e.g., 2, 3, 4, 5, 6, 7, 8, 9, 10, 11, 12, 13, 14, 15, or 16) GRN domains. In some embodiments, the PGRN comprises from 2 to 12 (e.g., 2, 3, 4, 5, 6, 7, 8, 9, 10, 11, or 12) GRN domains. In some embodiments, the PGRN comprises from 2 to 8 (e.g., 2, 3, 4, 5, 6, 7, or 8) GRN domains. In some embodiments, the PGRN comprises from 2 to 4 (e.g., 2, 3, or 4) GRN domains. In some embodiments, the PGRN comprises 2 GRN domains.

In some embodiments, the PGRN comprises a para-GRN domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 2. In some embodiments, the para-GRN domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 2. In some embodiments, the para-GRN domain has an amino acid sequence that is at least 95% (e.g., at least 95%, 96%, 97%, 98%, 99%, or more) identical to the amino acid sequence of SEQ ID NO. 2. In some embodiments, the para-GRN domain has an amino acid sequence of SEQ ID NO. 2.

In some embodiments, the PGRN comprises a GRN-1 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 3. In some embodiments, the GRN-1 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 3. In some embodiments, the GRN-1 domain has an amino acid sequence that is at least 95% (e.g., at least 95%, 96%, 97%, 98%, 99%, or more) identical to the amino acid sequence of SEQ ID NO. 3. In some embodiments, the GRN-1 domain has an amino acid sequence of SEQ ID NO. 3.

In some embodiments, the PGRN comprises a GRN-2 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 4. In some embodiments, the GRN-2 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 4. In some embodiments, the GRN-2 domain has an amino acid sequence that is at least 95% (e.g., at least 95%, 96%, 97%, 98%, 99%, or more) identical to the amino acid sequence of SEQ ID NO. 4. In some embodiments, the GRN-2 domain has an amino acid sequence of SEQ ID NO. 4.

In some embodiments, the PGRN comprises a GRN-3 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 5. In some embodiments, the GRN-3 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 5. In some embodiments, the GRN-3 domain has an amino acid sequence that is at least 95% (e.g., at least 95%, 96%, 97%, 98%, 99%, or more) identical to the amino acid sequence of SEQ ID NO. 5. In some embodiments, the GRN-3 domain has an amino acid sequence of SEQ ID NO. 5.

In some embodiments, the PGRN comprises a GRN-4 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 6. In some embodiments, the GRN-4 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 6. In some embodiments, the GRN-4 domain has an amino acid sequence that is at least 95% (e.g., at least 95%, 96%, 97%, 98%, 99%, or more) identical to the amino acid sequence of SEQ ID NO. 6. In some embodiments, the GRN-4 domain has an amino acid sequence of SEQ ID NO. 6.

In some embodiments, the PGRN comprises a GRN-5 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 7. In some embodiments, the GRN-5 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 7. In some embodiments, the GRN-5 domain has an amino acid sequence that is at least 95% (e.g., at least 95%, 96%, 97%, 98%, 99%, or more) identical to the amino acid sequence of SEQ ID NO. 7. In some embodiments, the GRN-5 domain has an amino acid sequence of SEQ ID NO. 7.

In some embodiments, the PGRN comprises a GRN-6 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 8. In some embodiments, the GRN-6 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 8. In some embodiments, the GRN-6 domain has an amino acid sequence that is at least 95% (e.g., at least 95%, 96%, 97%, 98%, 99%, or more) identical to the amino acid sequence of SEQ ID NO. 8. In some embodiments, the GRN-6 domain has an amino acid sequence of SEQ ID NO. 8.

In some embodiments, the PGRN comprises a GRN-7 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 9. In some embodiments, the GRN-7 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 9. In some embodiments, the GRN-7 domain has an amino acid sequence that is at least 95% (e.g., at least 95%, 96%, 97%, 98%, 99%, or more) identical to the amino acid sequence of SEQ ID NO. 9. In some embodiments, the GRN-7 domain has an amino acid sequence of SEQ ID NO. 9.

In some embodiments, the GRN is a full-length GRN, such as a GRN having any one of amino acid sequences of SEQ ID. NO 2-9 or a variant there of having at least 85% sequence identity thereto (e.g., at least 85%, 86%, 87%, 88%, 89%, 90%, 91%, 92%, 93%, 94%, 95%, 96%, 97%, 98%, 99%, or more sequence identity to any one of SEQ ID NOs. 2-9). In some embodiments, the GRN is a para-GRN or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 2. In some embodiments, the para-GRN has at least 90% (e.g., at least 90%, 91%, 92%, 93%, 94%, 95%, 96%, 97%, 98%, 99%, or more) sequence identity to SEQ ID NO. 2. In some embodiments, the para-GRN has at least 95% (e.g., at least 95%, 96%, 97%, 98%, 99%, or more) sequence identity to SEQ ID NO. 2. In some embodiments, the para-GRN has the amino acid sequence of SEQ ID NO. 2.

In some embodiments, the GRN is a GRN-1 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 3. In some embodiments, the GRN-1 has at least 90% (e.g., at least 90%, 91%, 92%, 93%, 94%, 95%, 96%, 97%, 98%, 99%, or more) sequence identity to SEQ ID NO. 3. In some embodiments, the GRN-1 has at least 95% (e.g., at least 95%, 96%, 97%, 98%, 99%, or more) sequence identity to SEQ ID NO. 3. In some embodiments, the GRN-1 has the amino acid sequence of SEQ ID NO. 3.

In some embodiments, the GRN is a GRN-2 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 4. In some embodiments, the GRN-2 has at least 90% (e.g., at least 90%, 91%, 92%, 93%, 94%, 95%, 96%, 97%, 98%, 99%, or more) sequence identity to SEQ ID NO. 4. In some embodiments, the GRN-2 has at least 95% (e.g., at least 95%, 96%, 97%, 98%, 99%, or more) sequence identity to SEQ ID NO. 4. In some embodiments, the GRN-2 has the amino acid sequence of SEQ ID NO. 4.

In some embodiments, the GRN is a GRN-3 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 5. In some embodiments, the GRN-3 has at least 90% (e.g., at least 90%, 91%, 92%, 93%, 94%, 95%, 96%, 97%, 98%, 99%, or more) sequence identity to SEQ ID NO. 5. In some embodiments, the GRN-3 has at least 95% (e.g., at least 95%, 96%, 97%, 98%, 99%, or more) sequence identity to SEQ ID NO. 5. In some embodiments, the GRN-3 has the amino acid sequence of SEQ ID NO. 5.

In some embodiments, the GRN is a GRN-4 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 6. In some embodiments, the GRN-4 has at least 90% (e.g., at least 90%, 91%, 92%, 93%, 94%, 95%, 96%, 97%, 98%, 99%, or more) sequence identity to SEQ ID NO. 6. In some embodiments, the GRN-4 has at least 95% (e.g., at least 95%, 96%, 97%, 98%, 99%, or more) sequence identity to SEQ ID NO. 6. In some embodiments, the GRN-4 has the amino acid sequence of SEQ ID NO. 6.

In some embodiments, the GRN is a GRN-5 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 7. In some embodiments, the GRN-5 has at least 90% (e.g., at least 90%, 91%, 92%, 93%, 94%, 95%, 96%, 97%, 98%, 99%, or more) sequence identity to SEQ ID NO. 7. In some embodiments, the GRN-5 has at least 95% (e.g., at least 95%, 96%, 97%, 98%, 99%, or more) sequence identity to SEQ ID NO. 7. In some embodiments, the GRN-5 has the amino acid sequence of SEQ ID NO. 7.

In some embodiments, the GRN is a GRN-6 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 8. In some embodiments, the GRN-6 has at least 90% (e.g., at least 90%, 91%, 92%, 93%, 94%, 95%, 96%, 97%, 98%, 99%, or more) sequence identity to SEQ ID NO. 8. In some embodiments, the GRN-6 has at least 95% (e.g., at least 95%, 96%, 97%, 98%, 99%, or more) sequence identity to SEQ ID NO. 8. In some embodiments, the GRN-6 has the amino acid sequence of SEQ ID NO. 8.

In some embodiments, the GRN is a GRN-7 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 9. In some embodiments, the GRN-7 has at least 90% (e.g., at least 90%, 91%, 92%, 93%, 94%, 95%, 96%, 97%, 98%, 99%, or more) sequence identity to SEQ ID NO. 9. In some embodiments, the GRN-7 has at least 95% (e.g., at least 95%, 96%, 97%, 98%, 99%, or more) sequence identity to SEQ ID NO. 9. In some embodiments, the GRN-7 has the amino acid sequence of SEQ ID NO. 9.

In some embodiments, the order of the GRN domains within the PGRN polypeptide occurs in the same order as observed in wild-type human PGRN. In some embodiments, the order of the GRN domains within the PGRN polypeptide occurs in an order different from the order found in wild-type human PGRN.

In some embodiments, the transgene encoding the PGRN or the GRN has been codon-optimized. In some embodiments, the codon-optimized transgene encoding the PGRN or the GRN contains a polynucleotide having at least 85% sequence identity (e.g., at least 85%, 90%, 95%, 96%, 97%, 98%, 99%, or more, sequence identity) to the nucleic acid sequence of SEQ ID NO. 19. In some embodiments, the transgene encoding the PGRN or the GRN includes a secretory signal peptide (e.g., a PGRN secretory signal peptide).

In some embodiments, the PGRN or the GRN is a PGRN or a GRN fusion protein. In some embodiments, the PGRN or the GRN fusion protein contains a low-density lipoprotein receptor family (LDLRf) binding (Rb) domain of apolipoprotein E (ApoE), or a fragment, variant, or oligomer thereof. In some embodiments, the Rb domain of ApoE, or a fragment, variant, or oligomer thereof, is operably linked to the N-terminus of the PGRN or the GRN. In some embodiments, the Rb domain of ApoE, or a fragment, variant, or oligomer thereof is operably linked to the C-terminus of the PGRN or the GRN. In some embodiments, the PGRN or the GRN fusion protein contains at least 1 (e.g., at least 1, 2, 3, 4, 5, 6, 7, 8, 9, 10, or more) oligomers of the Rb domain of ApoE. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 25-185 of SEQ ID NO. 11. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 50-180 of SEQ ID NO. 11. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 75-175 of SEQ ID NO. 11. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 100-170 of SEQ ID NO. 11. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 125-160 of SEQ ID NO. 11. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 130-150 of SEQ ID NO. 11. In some embodiments, the Rb domain contains a region of ApoE having at least 70% sequence identity (e.g., at least 70%, 75%, 80%, 85%, 90%, 95%, 96%, 97%, 98%, 99%, or greater, sequence identity) to residues 148-173 or a portion thereof containing residues 159-167 of SEQ ID NO. 11, or a variant having at least 70% sequence identity (e.g., at least 70%, 75%, 80%, 85%, 90%, 95%, 96%, 97%, 98%, 99%, or greater, sequence identity) to residues 159-167 of SEQ ID NO. 11). In some embodiments, the Rb domain contains a region having at least 70% sequence identity (e.g., at least 70%, 75%, 80%, 85%, 90%, 95%, 96%, 97%, 98%, 99%, or greater, sequence identity) to the amino acid sequence of residues 159-167 of SEQ ID NO. 11.

In some embodiments, the PGRN or GRN fusion protein contains PGRN or GRN and a glycosylation independent lysosomal targeting (GILT) tag. In some embodiments, the GILT tag is operably linked to the N-terminus of the PGRN or GRN. In some embodiments, the GILT tag is operably linked to the C-terminus of the PGRN or GRN. In some embodiments, the GILT tag contains a human IGF-II mutein having an amino acid sequence at least 70% identical to the amino acid sequence of mature human IGF-II (SEQ ID NO. 12). The mutein may have diminished binding affinity for the insulin receptor relative to the affinity of naturally-occurring human IGF-II for the insulin receptor, and/or may be resistant to furin cleavage. The mutein may bind to the human cation-independent mannose-6-phosphate receptor in a mannose-6-phosphate-independent manner. In some embodiments, the IGF-II mutein contains a mutation within a region corresponding to amino acids 30-40 of SEQ ID NO. 12, and wherein the mutation abolishes at least one furin protease cleavage site. In some embodiments, the mutation is an amino acid substitution, deletion, and/or insertion. In some embodiments, the mutation is a Lys or Ala amino acid substitution at a position corresponding to Arg37 or Arg40 of SEQ ID NO. 12. In some embodiments, the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO. 12, and combinations thereof. In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 13. In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 14. In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 15. In some embodiments, the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 16. In some embodiments, the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 17. In some embodiments, the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 18.

In some embodiments, the transgene encoding the PGRN or the GRN further comprises a micro RNA (miRNA) targeting sequence (e.g., a miR-126 targeting sequence). In some embodiments, the miRNA targeting sequence is located within the 3′-untranslated region (UTR) of the transgene.

In some embodiments, the PGRN or the GRN penetrates the blood-brain barrier (BBB) in the subject.

In some embodiments, the NCD is PGRN-associated NCD. In some embodiments, the FTLD or NCL is PGRN-associated FTLD or NCL.

In some embodiments, the subject suffering from PGRN-associated FTLD or NCL carries a mutation in the PGRN gene. The mutation in the PGRN gene may be a frameshift mutation. In some embodiments, the frameshift mutation is the p.C31LfsX35 mutation, the p.C31LfsX35 mutation, the p.S82VfsX174 mutation, the p.L271LfsX174 mutation, or the p.T382NfsX32 mutation.

Additionally or alternatively, the mutation in the PGRN gene may be, for example, a missense mutation. For example, the subject may carry the p.C521Y mutation, the p.A9D mutation, the p.P248L mutation, the p.R432C mutation, the p.C139R mutation, the p.C521Y mutation, or the p.C139R mutation.

Additionally or alternatively, the mutation in the PGRN gene may be, for example, a nonsense mutation. For example, the subject may carry the p.Q125X mutation. In some embodiments, the subject may carry the p.R493X mutation.

Additionally or alternatively, the mutation in the PGRN gene may be, for example, an insertion mutation. For example, the subject may carry the c.1145insA mutation.

Additionally or alternatively, the mutation in the PGRN gene may be, for example, a transversion mutation. For example, the subject may carry the p.0(IVS1+5G>C) mutation.

In some embodiments, the subject suffering from PGRN-associated FTLD or NCL may carry any other pathogenic mutation in the PGRN gene known to have a causative role in FTLD or NCL. For example, subjects having pathogenic mutations in the PGRN gene and that may be treated using the compositions and methods described herein include those that have any one of the mutations discussed in Gijselinck et al., Human Mutation 29(12), 1373-1386, (2012), the disclosure of which is incorporated herein by reference as it pertains to human PGRN mutations.

In some embodiments, the subject suffering from PGRN-associated FTLD may have the behavioral-variant frontotemporal dementia (BVFTD) variant of FTLD. In some embodiments, the subject suffering from PGRN-associated FTLD may have the semantic dementia (SD) variant of FTLD. In some embodiments, the subject suffering from PGRN-associated FTLD may have the progressive nonfluent aphasia (PNA) variant of FTLD.

In some embodiments, the subject suffering from PGRN-associated NCL may have the Santavuori-Haltia disease variant. In some embodiments, the subject suffering from PGRN-associated NCL may have the Batten disease variant. In some embodiments, the subject suffering from PGRN-associated NCL may have the Kuf's disease variant. In some embodiments, the subject suffering from PGRN-associated NCL may have the Jansky-Bielschowsky disease variant. In some embodiments, the subject suffering from PGRN-associated NCL may have the Finnish late infantile variant. In some embodiments, the subject suffering from PGRN-associated NCL may have the variant late infantile variant. In some embodiments, the subject suffering from PGRN-associated NCL may have the CLN7 variant. In some embodiments, the subject suffering from PGRN-associated NCL may have the CLN8 variant. In some embodiments, the subject suffering from PGRN-associated NCL may have the CLN10 variant. In some embodiments, the subject suffering from PG RN-associated NCL may have the CLN11 variant. In some embodiments, the subject suffering from PGRN-associated NCL may have the Turkish late infantile variant. In some embodiments, the subject suffering from PGRN-associated NCL may have the type 9 variant.

In some embodiments, the transgene encoding the PGRN or the GRN encodes a wild-type human PGRN or GRN (e.g., any one of SEQ ID NO. 1-9). In some embodiments, the transgene encoding the PGRN includes a polynucleotide encoding a polypeptide having at least 2 GRN domains (e.g., 2, 3, 4, 5, 6, 7, 8, or more GRN domains), such as the GRN domains having the amino acid sequence of any one of SEQ ID NOs. 2-9. In some embodiments, the transgene encoding the PGRN includes a polynucleotide encoding a polypeptide containing at least 2 GRN domains (e.g., 2, 3, 4, 5, 6, 7, 8, or more GRN domains) arranged in the same order as observed in the wild-type human PGRN. In some embodiments, the transgene encoding the PGRN includes a polynucleotide encoding a polypeptide containing at least 2 GRN domains (e.g., 2, 3, 4, 5, 6, 7, 8, or more GRN domains) arranged in an order distinct from the wild-type human PGRN. In some embodiments, the transgene encoding the PGRN or the GRN includes a polynucleotide encoding a polypeptide that contains at least 1 amino acid substitution, such as one or more conservative amino acid substitutions, relative to any of the polypeptides having the sequence of any one of SEQ ID NO. 1-8 (e.g., 1, 2, 3, 4, 5, 6, 7, 8, 9, or 10 or more amino acid substitutions, such as 1, 2, 3, 4, 5, 6, 7, 8, 9, or 10 or more conservative amino acid substitutions).

In some embodiments, the cells are pluripotent cells. In some embodiments, the pluripotent cells are ESCs. In some embodiments, the pluripotent cells are iPSCs. In some embodiments, the cells are CD34+ cells. In some embodiments, the cells are multipotent cells. In some embodiments, the multipotent cells are CD34+ cells. In some embodiments, the CD34+ cells are hematopoietic stem cells. In some embodiments, the CD34+ cells are myeloid progenitor cells. In some embodiments, the cells are blood line progenitor cells (BLPCs). In some embodiments, the BLPCs are monocytes. In some embodiments the cells are macrophages. In some embodiments, the cells are microglial progenitor cells. In some embodiments, the cells are microglia.

In some embodiments, a population of endogenous microglia in the subject has been ablated prior to administration of the composition to the subject. In some embodiments, the method includes ablating a population of endogenous microglia in the subject prior to administering the composition to the subject. In some embodiments, the microglia are ablated using an agent selected from the group consisting of busulfan, PLX3397, PLX647, PLX5622, treosulfan, and clodronate liposomes, by radiation therapy, or a combination thereof.

In some embodiments, the composition is administered systemically to the subject. In some embodiments, the composition is administered to the subject by way of intravenous injection. In some embodiments, the composition is administered directly to the central nervous system of the subject. In some embodiments, the composition is administered to the subject directly to the cerebrospinal fluid. For example, the composition may be administered to the subject by way of intracerebroventricular injection, intrathecal injection, stereotactic injection, or a combination thereof. In some embodiments, the composition may be administered to the subject by way of intraparenchymal injection.

In some embodiments, the composition is administered directly to the bone marrow of the subject, such as by way of intraosseous injection.

In some embodiments, the composition is administered to the subject by way of a bone marrow transplant.

In some embodiments, the composition is administered to the subject by way of intracerebroventricular injection. In some embodiments, the composition is administered to the subject by way of intravenous injection.

In some embodiments, the composition is administered to the subject by direct administration to the central nervous system of the subject and by systemic administration. In some embodiments, the composition is administered to the subject by way of intracerebroventricular injection and intravenous injection. In some embodiments, the composition is administered to the subject by way of intrathecal injection and intravenous injection. In some embodiments, the composition is administered to the subject by way intraparenchymal injection and intravenous injection.

In some embodiments, the method includes administering to the subject a population of cells. In some embodiments, the population of cells is administered to the subject prior to administration of the composition. In some embodiments, the population of cells is administered to the subject following administration of the composition. In some embodiments, the cells are selected from the group consisting of embryonic stem cells, induced pluripotent stem cells, hematopoietic stem cells, and myeloid progenitor cells. In some embodiments, the cells are not modified to express a transgene encoding the PGRN or the GRN. In some embodiments, the cells are administered to the subject systemically. In some embodiments, the cells are administered to the subject by way of intravenous injection.

In some embodiments, endogenous PGRN or GRN is disrupted in the cells prior to administration of the composition to the subject.

In some embodiments, the endogenous PGRN or GRN is disrupted by contacting the cells with a nuclease that catalyzes cleavage of the endogenous PGRN or GRN nucleic acid in the cells. In some embodiments, the nuclease is a CRISPR-associated protein. In some embodiments, the CRISPR-associated protein is CRISPR-associated protein 9. In some embodiments, the CRISPR-associated protein is CRISPR-associated protein 12a. In some embodiments, the nuclease is a transcription activator-like effector nuclease, a meganuclease, or a zinc finger nuclease.

In some embodiments, the endogenous PGRN or GRN is disrupted by contacting the cells with an inhibitory RNA molecule, e.g., for a time and in a quantity sufficient to disrupt expression of the endogenous PGRN or GRN. In some embodiments, the inhibitory RNA molecule is a short interfering RNA (siRNA), a short hairpin RNA (shRNA), or a miRNA.

In some embodiments, the endogenous PGRN or GRN is disrupted in the subject prior to administration of the composition to the subject. In some embodiments, the endogenous PGRN or GRN is disrupted by administering to the subject an inhibitory RNA molecule. In some embodiments, the inhibitory RNA molecule is a siRNA, a shRNA, or a miRNA. In some embodiments, the endogenous PGRN or GRN is disrupted in a population of neurons in the subject prior to administration of the composition to the subject. In some embodiments, the endogenous PGRN or GRN is disrupted in a population of neurons by contacting the population of neurons with an inhibitory RNA molecule, e.g., for a time and in a quantity sufficient to disrupt expression of the endogenous PGRN or GRN. In some embodiments, the inhibitory RNA molecule is a siRNA, a shRNA, or a miRNA.

In some embodiments, the cells are autologous cells. In some embodiments, the cells are allogeneic cells.

In some embodiments, the cells are transduced ex vivo to express the PGRN or the GRN.

In some embodiments, the cells are transduced with a viral vector selected from the group including an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

In some embodiments, the viral vector is a Retroviridae family viral vector. In some embodiments, the Retroviridae family viral vector is a lentiviral vector. In some embodiments, the Retroviridae family viral vector is an alpharetroviral vector. In some embodiments, the Retroviridae family viral vector is a gammaretroviral vector. In some embodiments, the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is an AAV selected from the group including AAV1, AAV2, AAV3, AAV4, AAV5, AAV6, AAV7, AAV8, AAV9, AAV10, and AAVrh74.

In some embodiments, the viral vector is a pseudotyped viral vector. In some embodiments, the viral vector is a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments, the cells are transfected ex vivo to express the PGRN or the GRN.

In some embodiments, the cells are transfected using an agent selected from the group including a cationic polymer, diethylaminoethyl-dextran, polyethylenimine, a cationic lipid, a liposome, calcium phosphate, an activated dendrimer, and a magnetic bead; or a technique selected from the group including electroporation, Nucleofection, squeeze-poration, sonoporation, optical transfection, Magnetofection, and impalefection.

In some embodiments, expression of the PGRN or the GRN in the cells is mediated using a ubiquitous promoter. Exemplary ubiquitous promoters are the elongation factor 1-alpha promoter and the phosphoglycerate kinase 1 promoter. In some embodiments, expression of the PGRN in the cells is mediated using a cell lineage-specific promoter. Exemplary cell lineage-specific promoters are the PGRN promoter, CD11 b promoter, CD68 promoter, C-X3-C motif chemokine receptor 1 promoter, allograft inflammatory factor 1 promoter, purinergic receptor P2Y12 promoter, transmembrane protein 119 promoter, and colony stimulating factor 1 receptor promoter.

In some embodiments, the composition is administered to the subject in an amount sufficient to increase the quantity of M2 microglia in the brain of the subject relative to the quantity of M1 microglia in the brain of the subject, decrease the level of one or more pro-inflammatory cytokines in the brain of the subject, increase the level of one or more anti-inflammatory cytokines in the brain of the subject, improve the cognitive performance of the subject, improve the motor function of the subject, reduce neuronal loss in the subject, and/or reduce levels of α-synuclein protein, tau protein, TAR DNA-binding protein 43 (TDP-43)-positive inclusions, fused in sarcoma (FUS)-positive inclusions, and/or ubiquitin-positive inclusions and inclusions, or aggregation thereof, in the subject.

In some embodiments, the subject is a human.

In another aspect, the disclosure provides a pharmaceutical composition containing a population of cells containing a transgene encoding a PGRN or a GRN.

In some embodiments of the preceding aspect, the cells are pluripotent cells. In some embodiments, the pluripotent cells are ESCs. In some embodiments, the pluripotent cells are iPSCs. In some embodiments, the cells are CD34+ cells. In some embodiments, the cells are multipotent cells. In some embodiments, the multipotent cells are CD34+ cells. In some embodiments, the CD34+ cells are hematopoietic stem cells. In some embodiments, the CD34+ cells are myeloid progenitor cells. In some embodiments, the cells are blood line progenitor cells (BLPCs). In some embodiments, the BLPCs are monocytes. In some embodiments the cells are macrophages. In some embodiments, the cells are microglial progenitor cells. In some embodiments, the cells are microglia.

In some embodiments, the cells are transduced ex vivo to express the PGRN or the GRN. In some embodiments, the cells are transfected ex vivo to express the PGRN or the GRN.

In some embodiments, the PGRN is a full-length PGRN, such as a PGRN having an amino acid sequence of SEQ ID NO. 1 or a variant thereof having at least 85% sequence identity thereto (e.g., having at least 85%, 86%, 87%, 88%, 89%, 90%, 91%, 92%, 93%, 94%, 95%, 96%, 97%, 98%, 99%, or more sequence identity to SEQ ID NO. 1). In some embodiments, the PGRN comprises at least 2 (e.g., at least 2, 3, 4, 5, 6, 7, 8 or more) GRN peptides having the amino acid sequence of any one of SEQ ID NOs. 2-9 or a variant thereof having at least 85% sequence identity thereto (e.g., at least 85%, 86%, 87%, 88%, 89%, 90%, 91%, 92%, 93%, 94%, 95%, 96%, 97%, 98%, 99%, or more sequence identity to any one of SEQ ID NOs. 2-9). In some embodiments, the PGRN comprises at least 2 (e.g., at least 2, 3, 4, 5, 6, 7, 8 or more) GRN domains. In some embodiments, the PGRN comprises at least 3 (e.g., at least 3, 4, 5, 6, 7, 8 or more) GRN domains. In some embodiments, the PGRN comprises at least 4 (e.g., at least 4, 5, 6, 7, 8 or more) GRN domains. In some embodiments, the PGRN comprises at least 5 (e.g., at least 5, 6, 7, 8 or more) GRN domains. In some embodiments, the PGRN comprises at least 6 (e.g., at least 6, 7, 8 or more) GRN domains. In some embodiments, the PGRN comprises at least 7 (e.g., at least 7, 8 or more) GRN domains. In some embodiments, the PGRN comprises at least 8 (e.g., at least 8 or more) GRN domains. In some embodiments, the PGRN comprises from 2 to 16 (e.g., 2, 3, 4, 5, 6, 7, 8, 9, 10, 11, 12, 13, 14, 15, or 16) GRN domains. In some embodiments, the PGRN comprises from 2 to 12 (e.g., 2, 3, 4, 5, 6, 7, 8, 9, 10, 11, or 12) GRN domains. In some embodiments, the PGRN comprises from 2 to 8 (e.g., 2, 3, 4, 5, 6, 7, or 8) GRN domains. In some embodiments, the PGRN comprises from 2 to 4 (e.g., 2, 3, or 4) GRN domains. In some embodiments, the PGRN comprises 2 GRN domains.

In some embodiments, the PGRN comprises a para-GRN domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 2. In some embodiments, the para-GRN domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 2. In some embodiments, the para-GRN domain has an amino acid sequence that is at least 95% (e.g., at least 95%, 96%, 97%, 98%, 99%, or more) identical to the amino acid sequence of SEQ ID NO. 2. In some embodiments, the para-GRN domain has an amino acid sequence of SEQ ID NO. 2.

In some embodiments, the PGRN comprises a GRN-1 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 3. In some embodiments, the GRN-1 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 3. In some embodiments, the GRN-1 domain has an amino acid sequence that is at least 95% (e.g., at least 95%, 96%, 97%, 98%, 99%, or more) identical to the amino acid sequence of SEQ ID NO. 3. In some embodiments, the GRN-1 domain has an amino acid sequence of SEQ ID NO. 3.

In some embodiments, the PGRN comprises a GRN-2 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 4. In some embodiments, the GRN-2 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 4. In some embodiments, the GRN-2 domain has an amino acid sequence that is at least 95% (e.g., at least 95%, 96%, 97%, 98%, 99%, or more) identical to the amino acid sequence of SEQ ID NO. 4. In some embodiments, the GRN-2 domain has an amino acid sequence of SEQ ID NO. 4.

In some embodiments, the PGRN comprises a GRN-3 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 5. In some embodiments, the GRN-3 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 5. In some embodiments, the GRN-3 domain has an amino acid sequence that is at least 95% (e.g., at least 95%, 96%, 97%, 98%, 99%, or more) identical to the amino acid sequence of SEQ ID NO. 5. In some embodiments, the GRN-3 domain has an amino acid sequence of SEQ ID NO. 5.

In some embodiments, the PGRN comprises a GRN-4 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 6. In some embodiments, the GRN-4 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 6. In some embodiments, the GRN-4 domain has an amino acid sequence that is at least 95% (e.g., at least 95%, 96%, 97%, 98%, 99%, or more) identical to the amino acid sequence of SEQ ID NO. 6. In some embodiments, the GRN-4 domain has an amino acid sequence of SEQ ID NO. 6.

In some embodiments, the PGRN comprises a GRN-5 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 7. In some embodiments, the GRN-5 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 7. In some embodiments, the GRN-5 domain has an amino acid sequence that is at least 95% (e.g., at least 95%, 96%, 97%, 98%, 99%, or more) identical to the amino acid sequence of SEQ ID NO. 7. In some embodiments, the GRN-5 domain has an amino acid sequence of SEQ ID NO. 7.

In some embodiments, the PGRN comprises a GRN-6 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 8. In some embodiments, the GRN-6 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 8. In some embodiments, the GRN-6 domain has an amino acid sequence that is at least 95% (e.g., at least 95%, 96%, 97%, 98%, 99%, or more) identical to the amino acid sequence of SEQ ID NO. 8. In some embodiments, the GRN-6 domain has an amino acid sequence of SEQ ID NO. 8.

In some embodiments, the PGRN comprises a GRN-7 domain having an amino acid sequence that is at least 85% (e.g., at least 85%, 86%, 87%, 88%, 89%, 90%, 91%, 92%, 93%, 94%, 95%, 96%, 97%, 98%, 99%, or more) identical to the amino acid sequence of SEQ ID NO. 9. In some embodiments, the GRN-7 domain has an amino acid sequence that is at least 90% (e.g., at least 90%, 91%, 92%, 93%, 94%, 95%, 96%, 97%, 98%, 99%, or more) identical to the amino acid sequence of SEQ ID NO. 9. In some embodiments, the GRN-7 domain has an amino acid sequence that is at least 95% (e.g., at least 95%, 96%, 97%, 98%, 99%, or more) identical to the amino acid sequence of SEQ ID NO. 9. In some embodiments, the GRN-7 domain has an amino acid sequence of SEQ ID NO. 9.

In some embodiments, the GRN is a full-length GRN, such as a GRN having any one of amino acid sequences of SEQ ID. NO 2-9 or a variant there of having at least 85% sequence identity thereto (e.g., at least 85%, 86%, 87%, 88%, 89%, 90%, 91%, 92%, 93%, 94%, 95%, 96%, 97%, 98%, 99%, or more sequence identity to any one of SEQ ID NOs. 2-9). In some embodiments, the GRN is a para-GRN or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 2. In some embodiments, the para-GRN has at least 90% (e.g., at least 90%, 91%, 92%, 93%, 94%, 95%, 96%, 97%, 98%, 99%, or more) sequence identity to SEQ ID NO. 2. In some embodiments, the para-GRN has at least 95% (e.g., at least 95%, 96%, 97%, 98%, 99%, or more) sequence identity to SEQ ID NO. 2. In some embodiments, the para-GRN has the amino acid sequence of SEQ ID NO. 2.

In some embodiments, the GRN is a GRN-1 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 3. In some embodiments, the GRN-1 has at least 90% (e.g., at least 90%, 91%, 92%, 93%, 94%, 95%, 96%, 97%, 98%, 99%, or more) sequence identity to SEQ ID NO. 3. In some embodiments, the GRN-1 has at least 95% (e.g., at least 95%, 96%, 97%, 98%, 99%, or more) sequence identity to SEQ ID NO. 3. In some embodiments, the GRN-1 has the amino acid sequence of SEQ ID NO. 3.

In some embodiments, the GRN is a GRN-2 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 4. In some embodiments, the GRN-2 has at least 90% (e.g., at least 90%, 91%, 92%, 93%, 94%, 95%, 96%, 97%, 98%, 99%, or more) sequence identity to SEQ ID NO. 4. In some embodiments, the GRN-2 has at least 95% (e.g., at least 95%, 96%, 97%, 98%, 99%, or more) sequence identity to SEQ ID NO. 4. In some embodiments, the GRN-2 has the amino acid sequence of SEQ ID NO. 4.

In some embodiments, the GRN is a GRN-3 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 5. In some embodiments, the GRN-3 has at least 90% (e.g., at least 90%, 91%, 92%, 93%, 94%, 95%, 96%, 97%, 98%, 99%, or more) sequence identity to SEQ ID NO. 5. In some embodiments, the GRN-3 has at least 95% (e.g., at least 95%, 96%, 97%, 98%, 99%, or more) sequence identity to SEQ ID NO. 5. In some embodiments, the GRN-3 has the amino acid sequence of SEQ ID NO. 5.

In some embodiments, the GRN is a GRN-4 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 6. In some embodiments, the GRN-4 has at least 90% (e.g., at least 90%, 91%, 92%, 93%, 94%, 95%, 96%, 97%, 98%, 99%, or more) sequence identity to SEQ ID NO. 6. In some embodiments, the GRN-4 has at least 95% (e.g., at least 95%, 96%, 97%, 98%, 99%, or more) sequence identity to SEQ ID NO. 6. In some embodiments, the GRN-4 has the amino acid sequence of SEQ ID NO. 6.

In some embodiments, the GRN is a GRN-5 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 7. In some embodiments, the GRN-5 has at least 90% (e.g., at least 90%, 91%, 92%, 93%, 94%, 95%, 96%, 97%, 98%, 99%, or more) sequence identity to SEQ ID NO. 7. In some embodiments, the GRN-5 has at least 95% (e.g., at least 95%, 96%, 97%, 98%, 99%, or more) sequence identity to SEQ ID NO. 7. In some embodiments, the GRN-5 has the amino acid sequence of SEQ ID NO. 7.

In some embodiments, the GRN is a GRN-6 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 8. In some embodiments, the GRN-6 has at least 90% (e.g., at least 90%, 91%, 92%, 93%, 94%, 95%, 96%, 97%, 98%, 99%, or more) sequence identity to SEQ ID NO. 8. In some embodiments, the GRN-6 has at least 95% (e.g., at least 95%, 96%, 97%, 98%, 99%, or more) sequence identity to SEQ ID NO. 8. In some embodiments, the GRN-6 has the amino acid sequence of SEQ ID NO. 8.

In some embodiments, the GRN is a GRN-7 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO. 9. In some embodiments, the GRN-7 has at least 90% (e.g., at least 90%, 91%, 92%, 93%, 94%, 95%, 96%, 97%, 98%, 99%, or more) sequence identity to SEQ ID NO. 9. In some embodiments, the GRN-7 has at least 95% (e.g., at least 95%, 96%, 97%, 98%, 99%, or more) sequence identity to SEQ ID NO. 9. In some embodiments, the GRN-7 has the amino acid sequence of SEQ ID NO. 9.

In some embodiments, the order of the GRN domains within the PGRN polypeptide occurs in the same order as observed in the wild-type human PGRN. In some embodiments, the order of the GRN domains within the PGRN polypeptide occurs in an order different from the order found in the wild-type human PGRN.

In some embodiments, the PGRN or the GRN comprises a secretory signal peptide. In some embodiments, the secretory signal peptide is a PGRN secretory signal peptide.

In some embodiments, the PGRN or the GRN is a PGRN or a GRN fusion protein. In some embodiments, the PGRN or the GRN fusion protein comprises an Rb domain of ApoE. In some embodiments, the Rb domain comprises a portion of ApoE having the amino acid sequence of residues 25-185, 50-180, 75-175, 100-170, 125-160, or 130-150 of SEQ ID NO. 11. In some embodiments, the Rb domain comprises a region having at least 70% sequence identity to the amino acid sequence of residues 159-167 of SEQ ID NO. 11.

In some embodiments, the transgene encoding the PGRN or the GRN further comprises a miRNA targeting sequence in the 3′-UTR. In some embodiments, the miRNA targeting sequence is a miR-126 targeting sequence.

In some embodiments, the endogenous PGRN or GRN is disrupted in the cells.

In some embodiments, the pharmaceutical composition is formulated for systemic administration to a subject. In some embodiments, the pharmaceutical composition is formulated for administration to a subject by way of intravenous injection. In some embodiments, the pharmaceutical composition is formulated for administration to the central nervous system of the subject. In some embodiments, the pharmaceutical composition is formulated for administration to the subject to the cerebrospinal fluid. In some embodiments, the pharmaceutical composition is formulated for administration to a subject by way of intracerebroventricular injection, intrathecal injection, stereotactic injection, or a combination thereof. In some embodiments, the pharmaceutical composition is formulated for administration to a subject by way of intraparenchymal injection. In some embodiments, the pharmaceutical composition is formulated for administration directly to the bone marrow of a subject. In some embodiments, the pharmaceutical composition is formulated for administration to a subject by way of intraosseous injection. In some embodiments, the pharmaceutical composition is formulated for administration to a subject by way of bone marrow transplant comprising the pharmaceutical composition. In some embodiments, the pharmaceutical composition is formulated for administration to a subject by way of intracerebroventricular injection and intravenous injection.

In some embodiments, the subject (e.g., a human) is diagnosed with an NCD. In some embodiments, the NCD is a major NCD. In some embodiments, the major NCD interferes with the subject's independence and/or normal daily functioning (e.g., social, occupational, or academic functioning, personal hygiene, grooming, dressing, toilet hygiene, functional mobility (e.g., ability to walk, get in and out of bed), and self-feeding). In some embodiments, the major NCD is associated with a score obtained by the subject on a cognitive test that is at least two standard deviations away from the mean score of a reference population. In some embodiments, the NCD is a mild NCD. In some embodiments, the mild NCD does not interfere with the subject's independence and/or normal daily functioning. In some embodiments, the mild NCD is associated with a score obtained by the subject on a cognitive test that is between one to two standard deviations away from the mean score of a reference population. In some embodiments, the cognitive test is selected from the group consisting of ADB, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression).

In some embodiments, the reference population is a general population. In some embodiments, the reference population is selected on the basis of the subject's age, medical history, education, socioeconomic status, and lifestyle. In some embodiments, the NCD is a frontotemporal NCD. In some embodiments, the frontotemporal NCD is FTLD. In some embodiments, the NCD is due to a lysosomal disease. In some embodiments, the lysosomal disease is NCL.

In an additional aspect, the disclosure provides kits containing compositions according to any of the above aspects and embodiments and a package insert. In some embodiments, the package insert instructs a user of the kit to perform a method according to any of the above aspects and embodiments.

Additional embodiments of the present invention are provided in the enumerated paragraphs below.

E1. A method of treating a subject diagnosed as having a neurocognitive disorder (NCD), the method comprising administering to the subject a composition comprising a population of cells containing a transgene encoding a progranulin (PGRN) or a granulin (GRN).
E2. The method of E1, wherein the NCD is a major NCD.
E3. The method of E2, wherein the major NCD interferes with the subject's independence and/or normal daily functioning.
E4. The method of E2 or E3, wherein the major NCD is associated with a score obtained by the subject on a cognitive test that is at least two standard deviations away from the mean score of a reference population.
E5. The method of E1, wherein the NCD is a mild NCD.
E6. The method of E5, wherein the mild NCD does not interfere with the subject's independence and/or normal daily functioning.
E7. The method of E5 or E6, wherein the mild NCD is associated with a score obtained by the subject on a cognitive test that is between one to two standard deviations away from the mean score of a reference population.
E8. The method of E4 or E7, wherein the reference population is a general population.
E9. The method of E4, E7, or E8, wherein the cognitive test is selected from the group consisting of Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE).
E10. The method of any one of E1-E9, wherein the NCD is associated with impairment in one or more of complex attention, executive function, learning and memory, language, perceptual-motor function, and social cognition.
E11. The method of any one of E1-E10, wherein the NCD is not due to delirium or other mental disorder.
E12. The method of any one of E1-E11, wherein the NCD is a frontotemporal NCD.
E13. The method of E12, wherein the frontotemporal NCD is frontotemporal lobar degeneration (FTLD).
E14. The method of any one of E1-E11, wherein the NCD is due to a lysosomal disease.
E15. The method of E14, wherein the lysosomal disease is neuronal ceroid lipofuscinosis (NCL).
E16. The method of any one of E1-E15, wherein the PGRN or the GRN comprises a secretory signal peptide.
E17. The method of E16, wherein the secretory signal peptide is a PGRN secretory signal peptide.
E18. The method of any one of E1-E17, wherein the cells contain a transgene encoding the PGRN.
E19. The method of E18, wherein the PGRN comprises at least 2 GRN domains.
E20. The method of E19, wherein the PGRN comprises at least 3 GRN domains.
E21. The method of E20, wherein the PGRN comprises at least 4 GRN domains.
E22. The method of E21, wherein the PGRN comprises at least 5 GRN domains.
E23. The method of E22, wherein the PGRN comprises at least 6 GRN domains.
E24. The method of E23, wherein the PGRN comprises at least 7 GRN domains.
E25. The method of E24, wherein the PGRN comprises at least 8 GRN domains.
E26. The method of any one of E1-E25, wherein the PGRN comprises from 2 to 16 GRN domains.
E27. The method of E26, wherein the PGRN comprises from 2 to 12 GRN domains.
E28. The method of E27, wherein the PGRN comprises from 2 to 8 GRN domains.
E29. The method of E28, wherein the PGRN comprises from 2 to 4 GRN domains.
E30. The method of E29, wherein the PGRN comprises 2 GRN domains.
E31. The method of any one of E1-E30, wherein the PGRN comprises a para-GRN domain having an amino acid sequence that is at least 85% identical to the amino acid sequence of SEQ ID NO. 2.
E32. The method of E31, wherein the para-GRN domain has an amino acid sequence that is at least 90% identical to the amino acid sequence of SEQ ID NO. 2.
E33. The method of E32, wherein the para-GRN domain has an amino acid sequence that is at least 95% identical to the amino acid sequence of SEQ ID NO. 2.
E34. The method of E33, wherein the para-GRN domain has an amino acid sequence of SEQ ID NO. 2.
E35. The method of any one of E1-E34, wherein the PGRN comprises a GRN-1 domain having an amino acid sequence that is at least 85% identical to the amino acid sequence of SEQ ID NO. 3.
E36. The method of E35, wherein the GRN-1 domain has an amino acid sequence that is at least 90% identical to the amino acid sequence of SEQ ID NO. 3.
E37. The method of E36, wherein the GRN-1 domain has an amino acid sequence that is at least 95% identical to the amino acid sequence of SEQ ID NO. 3.
E38. The method of E37, wherein the GRN-1 domain has the amino acid sequence of SEQ ID NO. 3.
E39. The method of any one of E1-E38, wherein the PGRN comprises a GRN-2 domain having an amino acid sequence that is at least 85% identical to the amino acid sequence of SEQ ID NO. 4.
E40. The method of E39, wherein the GRN-2 domain has an amino acid sequence that is at least 90% identical to the amino acid sequence of SEQ ID NO. 4.
E41. The method of E40, wherein the GRN-2 domain has an amino acid sequence that is at least 95% identical to the amino acid sequence of SEQ ID NO. 4.
E42. The method of E41, wherein the GRN-2 domain has an amino acid sequence of SEQ ID NO. 4.
E43. The method of any one of E1-E42, wherein the PGRN comprises a GRN-3 domain having an amino acid sequence that is at least 85% identical to the amino acid sequence of SEQ ID NO. 5.
E44. The method of E43, wherein the GRN-3 domain has an amino acid sequence that is at least 90% identical to the amino acid sequence of SEQ ID NO. 5.
E45. The method of E44, wherein the GRN-3 domain has an amino acid sequence that is at least 95% identical to the amino acid sequence of SEQ ID NO. 5.
E46. The method of E45, wherein the GRN-3 domain has an amino acid sequence of SEQ ID NO. 5.
E47. The method of any one of E1-E46, wherein the PGRN comprises a GRN-4 domain having an amino acid sequence that is at least 85% identical to the amino acid sequence of SEQ ID NO. 6.
E48. The method of E47, wherein the GRN-4 domain has an amino acid sequence that is at least 90% identical to the amino acid sequence of SEQ ID NO. 6.
E49. The method of E48, wherein the GRN-4 domain has an amino acid sequence that is at least 95% identical to the amino acid sequence of SEQ ID NO. 6.
E50. The method of E49, wherein the GRN-4 domain has an amino acid sequence of SEQ ID NO. 6.
E51. The method of any one of E1-E50, wherein the PGRN comprises a GRN-5 domain having an amino acid sequence that is at least 85% identical to the amino acid sequence of SEQ ID NO. 7.
E52. The method of E51, wherein the GRN-5 domain has an amino acid sequence that is at least 90% identical to the amino acid sequence of SEQ ID NO. 7.
E53. The method of E52, wherein the GRN-5 domain has an amino acid sequence that is at least 95% identical to the amino acid sequence of SEQ ID NO. 7.
E54. The method of E53, wherein the GRN-5 domain has an amino acid sequence of SEQ ID NO. 7.
E55. The method of any one of E1-E54, wherein the PGRN comprises a GRN-6 domain having an amino acid sequence that is at least 85% identical to the amino acid sequence of SEQ ID NO. 8.
E56. The method of E55, wherein the GRN-6 domain has an amino acid sequence that is at least 90% identical to the amino acid sequence of SEQ ID NO. 8.
E57. The method of E56, wherein the GRN-6 domain has an amino acid sequence that is at least 95% identical to the amino acid sequence of SEQ ID NO. 8.
E58. The method of E49, wherein the GRN-6 domain has an amino acid sequence of SEQ ID NO. 8.
E59. The method of any one of E1-E58, wherein the PGRN comprises a GRN-7 domain having an amino acid sequence that is at least 85% identical to the amino acid sequence of SEQ ID NO. 9.
E60. The method of E59, wherein the GRN-7 domain has an amino acid sequence that is at least 90% identical to the amino acid sequence of SEQ ID NO. 9.
E61. The method of E60, wherein the GRN-7 domain has an amino acid sequence that is at least 95% identical to the amino acid sequence of SEQ ID NO. 9.
E62. The method of E61, wherein the GRN-7 domain has an amino acid sequence of SEQ ID NO. 9.
E63. The method of any one of E1-E62, wherein the PGRN has an amino acid sequence that is at least 85% identical to the amino acid sequence of SEQ ID NO. 1.
E64. The method of E63, wherein the PGRN has an amino acid sequence that is at least 90% identical to the amino acid sequence of SEQ ID NO. 1.
E65. The method of E64, wherein the PGRN has an amino acid sequence that is at least 95% identical to the amino acid sequence of SEQ ID NO. 1.
E66. The method of E65, wherein the PGRN has an amino acid sequence of SEQ ID NO. 1.
E67. The method of any one of E1-E66, wherein the PGRN is a full-length PGRN.
E68. The method of any one of E1-E67, wherein the cells contain a transgene encoding the GRN.
E69. The method of any one of E1-E68, wherein the GRN is a para-GRN domain having an amino acid sequence that is at least 85% identical to the amino acid sequence of SEQ ID NO. 2.
E70. The method of E69, wherein the para-GRN domain has an amino acid sequence that is at least 90% identical to the amino acid sequence of SEQ ID NO. 2.
E71. The method of E70, wherein the para-GRN domain has an amino acid sequence that is at least 95% identical to the amino acid sequence of SEQ ID NO. 2.
E72. The method of E71, wherein the para-GRN domain has an amino acid sequence of SEQ ID NO. 2.
E73. The method of any one of E1-E72, wherein the GRN is a GRN-1 domain having an amino acid sequence that is at least 85% identical to the amino acid sequence of SEQ ID NO. 3.
E74. The method of E73, wherein the GRN-1 domain has an amino acid sequence that is at least 90% identical to the amino acid sequence of SEQ ID NO. 3.
E75. The method of E74, wherein the GRN-1 domain has an amino acid sequence that is at least 95% identical to the amino acid sequence of SEQ ID NO. 3.
E76. The method of E75, wherein the GRN-1 domain has an amino acid sequence of SEQ ID NO. 3.
E77. The method of any one of E1-E76, wherein the GRN is a GRN-2 domain having an amino acid sequence that is at least 85% identical to the amino acid sequence of SEQ ID NO. 4.
E78. The method of E77, wherein the GRN-2 domain has an amino acid sequence that is at least 90% identical to the amino acid sequence of SEQ ID NO. 4.
E79. The method of E78, wherein the GRN-2 domain has an amino acid sequence that is at least 95% identical to the amino acid sequence of SEQ ID NO. 4.
E80. The method of E79, wherein the GRN-2 domain has an amino acid sequence of SEQ ID NO. 4.
E81. The method of any one of E1-E80, wherein the GRN is a GRN-3 domain having an amino acid sequence that is at least 85% identical to the amino acid sequence of SEQ ID NO. 5.
E82. The method of E81, wherein the GRN-3 domain has an amino acid sequence that is at least 90% identical to the amino acid sequence of SEQ ID NO. 5.
E83. The method of E82, wherein the GRN-3 domain has an amino acid sequence that is at least 95% identical to the amino acid sequence of SEQ ID NO. 5.
E84. The method of E83, wherein the GRN-3 domain has an amino acid sequence of SEQ ID NO. 5.
E85. The method of any one of E1-E84, wherein the GRN is a GRN-4 domain having an amino acid sequence that is at least 85% identical to the amino acid sequence of SEQ ID NO. 6.
E86. The method of E85, wherein the GRN-4 domain has an amino acid sequence that is at least 90% identical to the amino acid sequence of SEQ ID NO. 6.
E87. The method of E86, wherein the GRN-4 domain has an amino acid sequence that is at least 95% identical to the amino acid sequence of SEQ ID NO. 6.
E88. The method of E87, wherein the GRN-4 domain has an amino acid sequence of SEQ ID NO. 6.
E89. The method of any one of E1-E88, wherein the GRN is a GRN-5 domain having an amino acid sequence that is at least 85% identical to the amino acid sequence of SEQ ID NO. 7.
E90. The method of E89, wherein the GRN-5 domain has an amino acid sequence that is at least 90% identical to the amino acid sequence of SEQ ID NO. 7.
E91. The method of E90, wherein the GRN-5 domain has an amino acid sequence that is at least 95% identical to the amino acid sequence of SEQ ID NO. 7.
E92. The method of E91, wherein the GRN-5 domain has an amino acid sequence of SEQ ID NO. 7.
E93. The method of any one of E1-E92, wherein the GRN is a GRN-6 domain having an amino acid sequence that is at least 85% identical to the amino acid sequence of SEQ ID NO. 8.
E94. The method of E93, wherein the GRN-6 domain has an amino acid sequence that is at least 90% identical to the amino acid sequence of SEQ ID NO. 8.
E95. The method of E94, wherein the GRN-6 domain has an amino acid sequence that is at least 95% identical to the amino acid sequence of SEQ ID NO. 8.
E96. The method of E95, wherein the GRN-6 domain has an amino acid sequence of SEQ ID NO. 8.
E97. The method of any one of E1-E96, wherein the GRN is a GRN-7 domain having an amino acid sequence that is at least 85% identical to the amino acid sequence of SEQ ID NO. 9.
E98. The method of E97, wherein the GRN-7 domain has an amino acid sequence that is at least 90% identical to the amino acid sequence of SEQ ID NO. 9.
E99. The method of E98, wherein the GRN-7 domain has an amino acid sequence that is at least 95% identical to the amino acid sequence of SEQ ID NO. 9.
E100. The method of E99, wherein the GRN-7 domain has an amino acid sequence of SEQ ID NO. 9.
E101. The method of any one of E1-E100, wherein the GRN comprises a full-length GRN.
E102. The method of any one of E1-E101, wherein the cells contain a PGRN transgene having at least 85% sequence identity to the nucleic acid sequence of SEQ ID NO. 10.
E103. The method of E102, wherein the cells contain a PGRN transgene having at least 90% sequence identity to the nucleic acid sequence of SEQ ID NO. 10.
E104. The method of E103, wherein the cells contain a PGRN transgene having at least 95% sequence identity to the nucleic acid sequence of SEQ ID NO. 10.
E105. The method of E104, wherein the cells contain a PGRN transgene having the nucleic acid sequence of SEQ ID NO. 10.
E106. The method of any one of E1-E105, wherein the PGRN or the GRN is a PGRN or a GRN fusion protein.
E107. The method of E106, wherein the PGRN or the GRN fusion protein comprises a receptor-binding (Rb) domain of apolipoprotein E (ApoE).
E108. The method of E107, wherein the Rb domain comprises a portion of ApoE having the amino acid sequence of residues 25-185, 50-180, 75-175, 100-170, 125-160, or 130-150 of SEQ ID NO. 11.
E109. The method of E107 or E108, wherein the Rb domain comprises a region having at least 70% sequence identity to the amino acid sequence of residues 159-167 of SEQ ID NO. 11.
E110. The method of any one of E1-E109, wherein the transgene encoding the PGRN or the GRN further comprises a micro RNA (miRNA) targeting sequence in the 3′-UTR.
E111. The method of E110, wherein the miRNA targeting sequence is a miR-126 targeting sequence.
E112. The method of any one of E1-E111, wherein upon administration of the composition to the subject, the PGRN or the GRN penetrates the blood-brain barrier in the subject.
E113. The method of any one of E13-E112, wherein the FTLD or NCL is PGRN-associated FTLD or NCL.
E114. The method of E113, wherein the PGRN-associated FTLD is the behavioral-variant frontotemporal dementia variant of FTLD.
E115. The method of E113, wherein the PGRN-associated FTLD is the semantic dementia variant of FTLD.
E116. The method of E113, wherein the PGRN-associated FTLD is the progressive nonfluent aphasia variant of FTLD.
E117. The method of E113, wherein the PGRN-associated NCL is Batten disease.
E118. The method of any one of E1-E117, wherein the cells are ESCs.
E119. The method of any one of E1-E117, wherein the cells are iPSCs) E120. The method of any one of E1-E117, wherein the cells are CD34+ cells.
E121. The method of E120, wherein the CD34+ cells are HSCs.
E122. The method of E120, wherein the CD34+ cells are MPCs.
E123. The method of any one of E1-E122, wherein a population of endogenous microglia in the subject has been ablated prior to administration of the composition.
E124. The method of any one of E1-E122, the method comprising ablating a population of endogenous microglia in the subject prior to administering the composition to the subject.
E125. The method of E123 or E124 wherein the microglia are ablated using an agent selected from the group consisting of busulfan, PLX3397, PLX647, PLX5622, treosulfan, and clodronate liposomes, by radiation therapy, or a combination thereof.
E126. The method of any one of E1-E125, wherein the composition is administered systemically to the subject.
E127. The method of E126, wherein the composition is administered to the subject by way of intravenous injection.
E128. The method of any one of E1-E125, wherein the composition is administered directly to the central nervous system of the subject.
E129. The method of E128, wherein the composition is administered to the subject by way of direct administration to the cerebrospinal fluid.
E130. The method of E128 or E129, wherein the composition is administered to the subject by way of intracerebroventricular injection, intrathecal injection, stereotactic injection, or a combination thereof.
E131. The method of E128, wherein the composition is administered to the subject by way of intraparenchymal injection.
E132. The method of any one of E1-E125, wherein the composition is administered directly to the bone marrow of the subject.
E133. The method of E132, wherein the composition is administered to the subject by way of intraosseous injection.
E134. The method of any one of E1-E125, wherein the composition is administered to the subject by way of a bone marrow transplant comprising the composition.
E135. The method of any one of E1-E125, wherein the composition is administered to the subject by way of intracerebroventricular injection.
E136. The method of any one of E1-E125, wherein the composition is administered to the subject by way of intrathecal injection.
E137. The method of any one of E1-E125, wherein the composition is administered to the subject by way of intraparenchymal injection.
E138. The method of any one of E1-E125, wherein the composition is administered to the subject by way of intravenous injection.
E139. The method of any one of E1-E125, wherein the composition is administered to the subject by direct administration to the central nervous system of the subject and by systemic administration.
E140. The method of E139, wherein the composition is administered to the subject by way of intracerebroventricular injection and intravenous injection.
E141. The method of E139, wherein the composition is administered to the subject by way of intrathecal injection and intravenous injection.
E142. The method of E139, wherein the composition is administered to the subject by way of intraparenchymal injection and intravenous injection.
E143. The method of any one of E1-E142, the method further comprising administering to the subject a population of cells.
E144. The method of E143, wherein the population of cells is administered to the subject prior to administration of the composition.
E145. The method of E143, wherein the population of cells is administered to the subject following administration of the composition.
E146. The method of any one of E143-E145, wherein the cells are selected from the group consisting of pluripotent cells, ESCs, IPSCs, multipotent cells, CD34+ cells, HSCs, MPCs, BLPCs, monocytes, macrophages, microglial progenitor cells, and microglia.
E147. The method of any one of E143-E146, wherein the cells are not modified to express a transgene encoding the PGRN or the GRN.
E148. The method of any one of E143-E147, wherein the cells are administered to the subject systemically.
E149. The method of E148, wherein the cells are administered to the subject by way of intravenous injection.
E150. The method of any one of E1-E149, wherein, prior to administration of the composition to the subject, endogenous PGRN or GRN is disrupted in the cells.
E151. The method of any one of E1-E150, wherein, prior to administration of the composition to the subject, the endogenous PGRN or GRN is disrupted in the subject.
E152. The method of E151, wherein, prior to the administration of the composition to the subject, the endogenous PGRN or GRN is disrupted in a population of neurons in the subject.
E153. The method of E150, wherein the endogenous PGRN or GRN is disrupted by contacting the cells with a nuclease that catalyzes cleavage of an endogenous PGRN or GRN nucleic acid in the cells.
E154. The method of E153, wherein the nuclease is a clustered regularly interspaced short palindromic repeats (CRISPR)-associated protein.
E155. The method of E154, wherein the CRISPR-associated protein is CRISPR-associated protein 9 (Cas9).
E156. The method of E154, wherein the CRISPR-associated protein is CRISPR-associated protein 12a (Cas12a) E157. The method of E153, wherein the nuclease is a transcription activator-like effector nuclease, a meganuclease, or a zinc finger nuclease.
E158. The method of any one of E150-E152, wherein the endogenous PGRN or GRN is disrupted by administering an inhibitory RNA molecule to the cells, the subject, or the population of neurons.
E159. The method of E158, wherein the inhibitory RNA molecule is a short interfering RNA, a short hairpin RNA, or a miRNA.
E160. The method of any one of E1-159, wherein the cells are autologous cells.
E161. The method of any one of E1-159, wherein the cells are allogeneic cells.
E162. The method of any one of E1-161, wherein the cells are transduced ex vivo to express the PGRN or the GRN.
E163. The method of E162, wherein the cells are transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.
E164. The method of E163, wherein the viral vector is a Retroviridae family viral vector.
E165. The method of E164, wherein the Retroviridae family viral vector is a lentiviral vector.
E166. The method of E164, wherein the Retroviridae family viral vector is an alpharetroviral vector.
E167. The method of E164, wherein the Retroviridae family viral vector is a gammaretroviral vector.
E168. The method of any one of E164-E167, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.
E169. The method of E163, wherein the viral vector is an AAV selected from the group consisting of AAV1, AAV2, AAV3, AAV4, AAV5, AAV6, AAV7, AAV8, AAV9, AAV10, and AAVrh74.
E170. The method of any one of E163-E169, wherein the viral vector is a pseudotyped viral vector.
E171. The method of E170, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.
E172. The method of any one of E1-E161, wherein the cells are transfected ex vivo to express the PGRN or the GRN.
E173. The method of E172, wherein the cells are transfected using: a) an agent selected from the group consisting of a cationic polymer, diethylaminoethyldextran, polyethylenimine, a cationic lipid, a liposome, calcium phosphate, an activated dendrimer, and a magnetic bead; or b) a technique selected from the group consisting of electroporation, Nucleofection, squeeze-poration, sonoporation, optical transfection, Magnetofection, and impalefection.
E174. The method of any one of E1-E173, wherein expression of the PGRN or the GRN in the cells is mediated by a ubiquitous promoter.
E175. The method of E174, wherein the ubiquitous promoter is selected from the group consisting of an elongation factor 1-alpha promoter and a phosphoglycerate kinase 1 promoter.
E176. The method of any one of E1-E173, wherein expression of the PGRN or the GRN is mediated by a cell lineage-specific promoter.
E177. The method of E176, wherein the cell lineage-specific promoter is selected from the group consisting of a PGRN promoter, CD11 b promoter, CD68 promoter, a C-X3-C motif chemokine receptor 1 promoter, an allograft inflammatory factor 1 promoter, a purinergic receptor P2Y12 promoter, a transmembrane protein 119 promoter, and a colony stimulating factor 1 receptor promoter.
E178. The method of any one of E1-E173, wherein expression of the PGRN or the GRN in the cells is mediated by a synthetic promoter.
E179. The method of any one of E1-E178, wherein the composition is administered to the subject in an amount sufficient to: a) increase the quantity of M2 microglia in the brain of the subject relative to the quantity of M1 microglia in the brain of the subject; b) decrease the level of one or more pro-inflammatory cytokines in the brain of the subject; c) increase the level of one or more anti-inflammatory cytokines in the brain of the subject; d) improve the cognitive performance of the subject; e) improve the motor function of the subject; f) reduce neuron loss in the subject; and/or g) reduce levels of α-synuclein protein, tau-positive neuronal inclusions, TAR DNA-binding protein 43 (TDP-43)-positive inclusions, fused in sarcoma (FUS)-positive inclusions, and/or ubiquitin-positive inclusions or aggregation thereof in the subject.
E180. The method of any one of E1-E179, wherein the subject is a human.
E181. A pharmaceutical composition comprising a population of cells containing a transgene encoding a PGRN or a GRN, the pharmaceutical composition further comprising one or more pharmaceutically acceptable carriers, diluent, or excipients.
E182. The pharmaceutical composition of E181, wherein the PGRN or the GRN comprises a secretory signal peptide.
E183. The pharmaceutical composition of E182, wherein the secretory signal peptide is a PGRN secretory signal peptide.
E184. The pharmaceutical composition of any one of E181-E183, wherein the cells contain a transgene encoding the PG RN.
E185. The pharmaceutical composition of E184, wherein the PGRN comprises at least 2 GRN domains.
E186. The pharmaceutical composition of E185, wherein the PG RN comprises at least 3 GRN domains.
E187. The pharmaceutical composition of E186, wherein the PG RN comprises at least 4 GRN domains.
E188. The pharmaceutical composition of E187, wherein the PG RN comprises at least 5 GRN domains.
E189. The pharmaceutical composition of E188, wherein the PG RN comprises at least 6 GRN domains.
E190. The pharmaceutical composition of E189, wherein the PG RN comprises at least 7 GRN domains.
E191. The pharmaceutical composition of E190, wherein the PGRN comprises at least 8 GRN domains.
E192. The pharmaceutical composition of any one of E181-E191, wherein the PGRN comprises from 2 to 16 GRN domains.
E193. The pharmaceutical composition of E192, wherein the PG RN comprises from 2 to 12 GRN domains.
E194. The pharmaceutical composition of E193, wherein the PG RN comprises from 2 to 8 GRN domains.
E195. The pharmaceutical composition of E194, wherein the PG RN comprises from 2 to 4 GRN domains.
E196. The pharmaceutical composition of E195, wherein the PG RN comprises 2 GRN domains.
E197. The pharmaceutical composition of any one of E181-E196, wherein the PGRN comprises a para-GRN domain having an amino acid sequence that is at least 85% identical to the amino acid sequence of SEQ ID NO. 2.
E198. The pharmaceutical composition of E197, wherein the para-GRN domain has an amino acid sequence that is at least 90% identical to the amino acid sequence of SEQ ID NO. 2.
E199. The pharmaceutical composition of E198, wherein the para-GRN domain has an amino acid sequence that is at least 95% identical to the amino acid sequence of SEQ ID NO. 2.
E200. The pharmaceutical composition of E199, wherein the para-GRN domain has an amino acid sequence of SEQ ID NO. 2.
E201. The pharmaceutical composition of any one of E181-E200, wherein the PGRN comprises a GRN-1 domain having an amino acid sequence that is at least 85% identical to the amino acid sequence of SEQ ID NO. 3.
E202. The pharmaceutical composition of E201, wherein the GRN-1 domain has an amino acid sequence that is at least 90% identical to the amino acid sequence of SEQ ID NO. 3.
E203. The pharmaceutical composition of E202, wherein the GRN-1 domain has an amino acid sequence that is at least 95% identical to the amino acid sequence of SEQ ID NO. 3.
E204. The pharmaceutical composition of E203, wherein the GRN-1 domain has the amino acid sequence of SEQ ID NO. 3.
E205. The pharmaceutical composition of any one of E181-E204, wherein the PGRN comprises a GRN-2 domain having an amino acid sequence that is at least 85% identical to the amino acid sequence of SEQ ID NO. 4.
E206. The pharmaceutical composition of E205, wherein the GRN-2 domain has an amino acid sequence that is at least 90% identical to the amino acid sequence of SEQ ID NO. 4.
E207. The pharmaceutical composition of E206, wherein the GRN-2 domain has an amino acid sequence that is at least 95% identical to the amino acid sequence of SEQ ID NO. 4.
E208. The pharmaceutical composition of E207, wherein the GRN-2 domain has an amino acid sequence of SEQ ID NO. 4.
E209. The pharmaceutical composition of any one of E181-E208, wherein the PGRN comprises a GRN-3 domain having an amino acid sequence that is at least 85% identical to the amino acid sequence of SEQ ID NO. 5.
E210. The pharmaceutical composition of E209, wherein the GRN-3 domain has an amino acid sequence that is at least 90% identical to the amino acid sequence of SEQ ID NO. 5.
E211. The pharmaceutical composition of E210, wherein the GRN-3 domain has an amino acid sequence that is at least 95% identical to the amino acid sequence of SEQ ID NO. 5.
E212. The pharmaceutical composition of E211, wherein the GRN-3 domain has an amino acid sequence of SEQ ID NO. 5.
E213. The pharmaceutical composition of any one of E181-E212, wherein the PGRN comprises a GRN-4 domain having an amino acid sequence that is at least 85% identical to the amino acid sequence of SEQ ID NO. 6.
E214. The pharmaceutical composition of E213, wherein the GRN-4 domain has an amino acid sequence that is at least 90% identical to the amino acid sequence of SEQ ID NO. 6.
E215. The pharmaceutical composition of E214, wherein the GRN-4 domain has an amino acid sequence that is at least 95% identical to the amino acid sequence of SEQ ID NO. 6.
E216. The pharmaceutical composition of E215, wherein the GRN-4 domain has an amino acid sequence of SEQ ID NO. 6.
E217. The pharmaceutical composition of any one of E181-E216, wherein the PGRN comprises a GRN-5 domain having an amino acid sequence that is at least 85% identical to the amino acid sequence of SEQ ID NO. 7.
E218. The pharmaceutical composition of E217, wherein the GRN-5 domain has an amino acid sequence that is at least 90% identical to the amino acid sequence of SEQ ID NO. 7.
E219. The pharmaceutical composition of E218, wherein the GRN-5 domain has an amino acid sequence that is at least 95% identical to the amino acid sequence of SEQ ID NO. 7.
E220. The pharmaceutical composition of E219, wherein the GRN-5 domain has an amino acid sequence of SEQ ID NO. 7.
E221. The pharmaceutical composition of any one of E181-E220, wherein the PGRN comprises a GRN-6 domain having an amino acid sequence that is at least 85% identical to the amino acid sequence of SEQ ID NO. 8.
E222. The pharmaceutical composition of E221, wherein the GRN-6 domain has an amino acid sequence that is at least 90% identical to the amino acid sequence of SEQ ID NO. 8.
E223. The pharmaceutical composition of E222, wherein the GRN-6 domain has an amino acid sequence that is at least 95% identical to the amino acid sequence of SEQ ID NO. 8.
E224. The pharmaceutical composition of E223, wherein the GRN-6 domain has an amino acid sequence of SEQ ID NO. 8.
E225. The pharmaceutical composition of any one of E181-E224, wherein the PGRN comprises a GRN-7 domain having an amino acid sequence that is at least 85% identical to the amino acid sequence of SEQ ID NO. 9.
E226. The pharmaceutical composition of E225, wherein the GRN-7 domain has an amino acid sequence that is at least 90% identical to the amino acid sequence of SEQ ID NO. 9.
E227. The pharmaceutical composition of E226, wherein the GRN-7 domain has an amino acid sequence that is at least 95% identical to the amino acid sequence of SEQ ID NO. 9.
E228. The pharmaceutical composition of E226, wherein the GRN-7 domain has an amino acid sequence of SEQ ID NO. 9.
E229. The pharmaceutical composition of any one of E181-E228, wherein the PGRN is a full-length PGRN.
E230. The pharmaceutical composition of E181-E229, wherein the PG RN has an amino acid sequence that is at least 85% identical to the amino acid sequence of SEQ ID NO. 1.
E231. The pharmaceutical composition of E230, wherein the PG RN has an amino acid sequence that is at least 90% identical to the amino acid sequence of SEQ ID NO. 1.
E232. The pharmaceutical composition of E231, wherein the PG RN has an amino acid sequence that is at least 95% identical to the amino acid sequence of SEQ ID NO. 1.
E233. The pharmaceutical composition of E232, wherein the PG RN has an amino acid sequence of SEQ ID NO. 1.
E234. The pharmaceutical composition of any one of E181-E233, wherein the cells contain a transgene encoding the GRN.
E235. The pharmaceutical composition of any one of E181-E234, wherein the GRN is a para-GRN domain having an amino acid sequence that is at least 85% identical to the amino acid sequence of SEQ ID NO. 2.
E236. The pharmaceutical composition of E235, wherein the para-GRN domain has an amino acid sequence that is at least 90% identical to the amino acid sequence of SEQ ID NO. 2.
E237. The pharmaceutical composition of E236, wherein the para-GRN domain has an amino acid sequence that is at least 95% identical to the amino acid sequence of SEQ ID NO. 2.
E238. The pharmaceutical composition of E237, wherein the para-GRN domain has an amino acid sequence of SEQ ID NO. 2.
E239. The pharmaceutical composition of any one of E181-E238, wherein the GRN is a GRN-1 domain having an amino acid sequence that is at least 85% identical to the amino acid sequence of SEQ ID NO. 3.
E240. The pharmaceutical composition of E239, wherein the GRN-1 domain has an amino acid sequence that is at least 90% identical to the amino acid sequence of SEQ ID NO. 3.
E241. The pharmaceutical composition of E240, wherein the GRN-1 domain has an amino acid sequence that is at least 95% identical to the amino acid sequence of SEQ ID NO. 3.
E242. The pharmaceutical composition of E241, wherein the GRN-1 domain has an amino acid sequence of SEQ ID NO. 3.
E243. The pharmaceutical composition of any one of E181-E242, wherein the GRN is a GRN-2 domain having an amino acid sequence that is at least 85% identical to the amino acid sequence of SEQ ID NO. 4.
E244. The pharmaceutical composition of E243, wherein the GRN-2 domain has an amino acid sequence that is at least 90% identical to the amino acid sequence of SEQ ID NO. 4.
E245. The pharmaceutical composition of E244, wherein the GRN-2 domain has an amino acid sequence that is at least 95% identical to the amino acid sequence of SEQ ID NO. 4.
E246. The pharmaceutical composition of E245, wherein the GRN-2 domain has an amino acid sequence of SEQ ID NO. 4.
E247. The pharmaceutical composition of any one of E181-E246, wherein the GRN is a GRN-3 domain having an amino acid sequence that is at least 85% identical to the amino acid sequence of SEQ ID NO. 5.
E248. The pharmaceutical composition of E247, wherein the GRN-3 domain has an amino acid sequence that is at least 90% identical to the amino acid sequence of SEQ ID NO. 5.
E249. The pharmaceutical composition of E248, wherein the GRN-3 domain has an amino acid sequence that is at least 95% identical to the amino acid sequence of SEQ ID NO. 5.
E250. The pharmaceutical composition of E249, wherein the GRN-3 domain has an amino acid sequence of SEQ ID NO. 5.
E251. The pharmaceutical composition of any one of E181-E250, wherein the GRN-4 domain has an amino acid sequence that is at least 85% identical to the amino acid sequence of SEQ ID NO. 6.
E252. The pharmaceutical composition of E251, wherein the GRN-4 domain has an amino acid sequence that is at least 90% identical to the amino acid sequence of SEQ ID NO. 6.
E253. The pharmaceutical composition of E252, wherein the GRN-4 domain has an amino acid sequence that is at least 95% identical to the amino acid sequence of SEQ ID NO. 6.
E254. The pharmaceutical composition of E253, wherein the GRN-4 domain has an amino acid sequence of SEQ ID NO. 6.
E255. The pharmaceutical composition of any one of E181-E254, wherein the GRN is a GRN-5 domain having an amino acid sequence that is at least 85% identical to the amino acid sequence of SEQ ID NO. 7.
E256. The pharmaceutical composition of E255, wherein the GRN-5 domain has an amino acid sequence that is at least 90% identical to the amino acid sequence of SEQ ID NO. 7.
E257. The pharmaceutical composition of E256, wherein the GRN-5 domain has an amino acid sequence that is at least 95% identical to the amino acid sequence of SEQ ID NO. 7.
E258. The pharmaceutical composition of E257, wherein the GRN-5 domain has an amino acid sequence of SEQ ID NO. 7.
E259. The pharmaceutical composition of any one of E181-E258, wherein the GRN is a GRN-6 domain having an amino acid sequence that is at least 85% identical to the amino acid sequence of SEQ ID NO. 8.
E260. The pharmaceutical composition of E259, wherein the GRN-6 domain has an amino acid sequence that is at least 90% identical to the amino acid sequence of SEQ ID NO. 8.
E261. The pharmaceutical composition of E260, wherein the GRN-6 domain has an amino acid sequence that is at least 95% identical to the amino acid sequence of SEQ ID NO. 8.
E262. The pharmaceutical composition of E261, wherein the GRN-6 domain has an amino acid sequence of SEQ ID NO. 8.
E263. The pharmaceutical composition of any one of E181-E262, wherein the GRN is a GRN-7 domain having an amino acid sequence that is at least 85% identical to the amino acid sequence of SEQ ID NO. 9.
E264. The pharmaceutical composition of E263, wherein the GRN-7 domain has an amino acid sequence that is at least 90% identical to the amino acid sequence of SEQ ID NO. 9.
E265. The pharmaceutical composition of E264, wherein the GRN-7 domain has an amino acid sequence that is at least 95% identical to the amino acid sequence of SEQ ID NO. 9.
E266. The pharmaceutical composition of E265, wherein the GRN-7 domain has an amino acid sequence of SEQ ID NO. 9.
E267. The pharmaceutical composition of any one of E181-E266, wherein the GRN is a full-length GRN.
E268. The pharmaceutical composition of any one of E181-E267, wherein the cells contain a PG RN transgene having at least 85% sequence identity to the nucleic acid sequence of SEQ ID NO. 10.
E269. The pharmaceutical composition of E268, wherein the PG RN transgene has at least 90% sequence identity to the nucleic acid sequence of SEQ ID NO. 10.
E270. The pharmaceutical composition of E269, wherein the PG RN transgene has at least 95% sequence identity to the nucleic acid sequence of SEQ ID NO. 10.
E271. The pharmaceutical composition of E270, wherein the PG RN transgene has the nucleic acid sequence of SEQ ID NO. 10.
E272. The pharmaceutical composition of any one of E181-E271, wherein the PGRN or the GRN is a PGRN or a GRN fusion protein.
E273. The pharmaceutical composition of E272, wherein the PG RN or the GRN fusion protein comprises a Rb domain of ApoE.
E274. The pharmaceutical composition of E273, wherein the Rb domain comprises a portion of ApoE having the amino acid sequence of residues 25-185, 50-180, 75-175, 100-170, 125-160, or 130-150 of SEQ ID NO. 11.
E275. The pharmaceutical composition of E273 or E274, wherein the Rb domain comprises a region having at least 70% sequence identity to the amino acid sequence of residues 159-167 of SEQ ID NO.
E276. The pharmaceutical composition of any one of E181-E275, wherein the transgene encoding PGRN or GRN further comprises a miRNA targeting sequence in the 3′-UTR.
E277. The pharmaceutical composition of E276, wherein the miRNA targeting sequence is a miR-126 targeting sequence.
E278. The pharmaceutical composition of any one of E181-E277, wherein the cells are ESCs.
E279. The pharmaceutical composition of any one of E181-E277, wherein the cells are iPSCs.
E280. The pharmaceutical composition of any one of E181-E277, wherein the cells are CD34+ cells.
E281. The pharmaceutical composition of E280, wherein the CD34+ cells are HSCs.
E282. The pharmaceutical composition of E280, wherein the CD34+ cells are MPCs.
E283. The pharmaceutical composition of any one of E181-E282, wherein the cells are transfected ex vivo to express the PG RN or the GRN.
E284. The pharmaceutical composition of any one of E181-E282, wherein the cells are transduced ex vivo to express the PG RN or the GRN.
E285. The pharmaceutical composition of any one of E181-E284, wherein the pharmaceutical composition is formulated for systemic administration to a human subject.
E286. The pharmaceutical composition of E285, wherein the pharmaceutical composition is formulated for administration to a human subject by way of intravenous injection.
E287. The pharmaceutical composition of any one of E181-E284, wherein the pharmaceutical composition is formulated for administration to a human subject directly to the nervous system of the subject.
E288. The pharmaceutical composition of E287, wherein the pharmaceutical composition is formulated for administration to a human subject to the cerebrospinal fluid.
E289. The pharmaceutical composition of E287 or E288, wherein the pharmaceutical composition is formulated for administration to a human subject by way of intracerebroventricular injection, intrathecal injection, stereotactic injection, or a combination thereof.
E290. The pharmaceutical composition of E287, wherein the pharmaceutical composition is formulated for administration to a human subject by way of intraparenchymal injection.
E291. The pharmaceutical composition of any one of E181-E284, wherein the pharmaceutical composition is formulated for administration directly to the bone marrow of a human subject.
E292. The pharmaceutical composition of E291, wherein the pharmaceutical composition is formulated for administration to a human subject by way of intraosseous injection.
E293. The pharmaceutical composition of any one of E181-E284, wherein the pharmaceutical composition is formulated for administration to a human subject by way of a bone marrow transplant comprising the composition.
E294. The pharmaceutical composition of any one of E181-E284, wherein the pharmaceutical composition is formulated for systemic administration to a human subject and for administration directly the central nervous system of a human subject.
E295. The pharmaceutical composition of E294, wherein the pharmaceutical composition is formulated for administration by way of intracerebroventricular injection and intravenous injection.
E296. The pharmaceutical composition of E294, wherein the pharmaceutical composition is formulated for administration by way of intrathecal injection and intravenous injection.
E297. The pharmaceutical composition of E294, wherein the pharmaceutical composition is formulated for administration by way of intraparenchymal injection and intravenous injection.
E298. The pharmaceutical composition of any one of E285-E297, wherein the human subject is diagnosed with an NCD.
E299. The pharmaceutical composition of E298, wherein the NCD is a major NCD.
E300. The pharmaceutical composition of E299, wherein the major NCD interferes with the subject's independence and/or normal daily functioning.
E301. The pharmaceutical composition of E299 or E300, wherein the major NCD is associated with a score obtained by the subject on a cognitive test that is at least two standard deviations away from the mean score of a reference population.
E302. The pharmaceutical composition of E298, wherein the NCD is a mild NCD.
E303. The pharmaceutical composition of E302, wherein the mild NCD does not interfere with the subject's independence and/or normal daily functioning.
E304. The pharmaceutical composition of E302 or E303, wherein the mild NCD is associated with a score obtained by the subject on a cognitive test that is between one to two standard deviations away from the mean score of a reference population.
E305. The pharmaceutical composition of E301 or E304, wherein the reference population is a general population.
E306. The pharmaceutical composition of E301, E304, or E305, wherein the cognitive test is selected from the group consisting of AD8, AWV, GPCOG, HRA, MIS, MMSE, MoCA, SLUMS, and Short IQCODE.
E307. The pharmaceutical composition of any one of E298-E306, wherein the NCD is associated with impairment in one or more of complex attention, executive function, learning and memory, language, perceptual-motor function, and social cognition.
E308. The pharmaceutical composition of any one of E298-E307, wherein the NCD is not due to delirium or other mental disorder.
E309. The pharmaceutical composition of any one of E298-E308, wherein the NCD is a frontotemporal NCD.
E310. The pharmaceutical composition of E309, wherein the frontotemporal NCD is FTLD.
E311. The pharmaceutical composition of any one of E298-E308, wherein the NCD is due to a lysosomal disease.
E312. The pharmaceutical composition of E311, wherein the lysosomal disease is NCL.
E313. A kit comprising the pharmaceutical composition of any one of E181-E312 and a package insert.
E314. The kit of E313, wherein the package insert instructs a user of the kit to perform the method of any one of E1-E180.
E315. The method of any one of E1-E180, wherein the PGRN or GRN fusion protein comprises PGRN or GRN and a GILT tag.
E316. The method of E315, wherein the GILT tag is operably linked to the N-terminus of the PGRN or GRN.
E317. The method of E315, wherein the GILT tag is operably linked to the C-terminus of the PGRN or GRN.
E318. The method of any one of E315-317, wherein the GILT tag contains a human IGF-II mutein having an amino acid sequence at least 70% identical to the amino acid sequence of mature human IGF-II (SEQ ID NO. 12).
E319. The method of any one of E315-318, wherein the IGF-II mutein contains a mutation within a region corresponding to amino acids 30-40 of SEQ ID NO. 12, and wherein the mutation abolishes at least one furin protease cleavage site.
E320. The method of E319, wherein the mutation is an amino acid substitution, deletion, and/or insertion.
E321. The method of E320, wherein the mutation is a Lys or Ala amino acid substitution at a position corresponding to Arg37 or Arg40 of SEQ ID NO. 12.
E322. The method of E320, wherein the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO. 12, and combinations thereof.
E323. The method of any one of E315-E322, wherein the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 13.
E324. The method of any one of E315-E322, wherein the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 14
E325. The method of any one of E315-E322, wherein the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 15.
E326. The method of any one of E315-E322, wherein the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 16.
E327. The method of any one of E315-E322, wherein the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 17.
E328. The method of any one of E315-E322, wherein the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 18.
E329. The method of any one of E1-E180 or E315-E328, wherein the cells are pluripotent cells (e.g., ESCs, iPSCs), multipotent cells (e.g., CD34+ cells, such as, e.g., HSCs or MPCs), BLPCs, monocytes, macrophages, microglial progenitor cells, or microglia.
E330. The method of any one of E1-E180 or E315-E329, wherein the transgene is capable of expression in a macrophage or a microglial cell.
E331. The method of any one of E1-E180 or E315-E330, wherein the transgene is a codon-optimized transgene.
E332. The method of E331, wherein the codon-optimized transgene includes a polynucleotide having at least 85% sequence identity to the nucleic acid sequence of SEQ ID NO. 19.
E333. The composition of any one of E181-E312, wherein the PGRN or GRN fusion protein comprises PGRN or GRN and a GILT tag.
E334. The composition of E333, wherein the GILT tag is operably linked to the N-terminus of the PGRN or GRN.
E335. The composition of E333, wherein the GILT tag is operably linked to the C-terminus of the PGRN or GRN.
E336. The composition of any one of E333-E335, wherein the GILT tag contains a human IGF-II mutein having an amino acid sequence at least 70% identical to the amino acid sequence of mature human IGF-II (SEQ ID NO. 12).
E337. The composition of any one of E333-E336, wherein the IGF-II mutein contains a mutation within a region corresponding to amino acids 30-40 of SEQ ID NO. 12, and wherein the mutation abolishes at least one furin protease cleavage site.
E338. The composition of E337, wherein the mutation is an amino acid substitution, deletion, and/or insertion.
E339. The composition of E338, wherein the mutation is a Lys or Ala amino acid substitution at a position corresponding to Arg37 or Arg40 of SEQ ID NO. 12.
E340. The composition of E338, wherein the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO. 12, and combinations thereof.
E341. The composition of any one of E333-E340, wherein the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 13.
E342. The composition of any one of E333-E340, wherein the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 14
E343. The composition of any one of E333-E340, wherein the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 15.
E344. The composition of any one of E333-E340, wherein the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 16.
E345. The composition of any one of E333-E340, wherein the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 17.
E346. The composition of any one of E333-E340, wherein the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 18.
E347. The composition of any one of E181-E312 or E333-E346, wherein the cells are pluripotent cells (e.g., ESCs, iPSCs), multipotent cells (e.g., CD34+ cells, such as, e.g., HSCs or MPCs), BLPCs, monocytes, macrophages, microglial progenitor cells, or microglia.
E348. The composition of any one of E181-E312 or E333-E347, wherein the transgene is capable of expression in a macrophage or a microglial cell.
E349. The composition of any one of E1-E180 or E333-E348, wherein the transgene is a codon-optimized transgene.
E350. The composition of E349, wherein the codon-optimized transgene includes a polynucleotide having at least 85% sequence identity to the nucleic acid sequence of SEQ ID NO. 19.

### BRIEF DESCRIPTION OF THE DRAWINGS

FIGS. 1A-1B are a series of plots showing transduction of human cells with a lentiviral vector containing a transgene encoding the human progranulin (PGRN) protein. Cell lysates were generated from human 239T cells transduced with a lentiviral vector encoding PGRN (MND.GRN) or green fluorescent protein (GFP; MND.GFP) at a multiplicity of infection (MOI) of 10, 50, 100, or 200. A separate set of control cells were not transduced (NTC). Densitometry was used to quantify PGRN levels over actin (FIG. 1A). Western blots using an antibody raised against human PGRN indicate stable PGRN expression in 239T cells, with highest expression observed at MOI 200 (FIG. 1B). All groups were showed statistically significant differences, except for the NTC cells and MOI 10 GFP cells. Statistical analysis was performed using ANOVA.

FIG. 2 is a Western blot showing expression of human PGRN in murine lineage negative (Lin−) cells transduced with a lentiviral vector containing a transgene encoding human PGRN (i.e., a MND.GRN vector). Conditioned media generated from Lin− mouse cells non-transduced (−) or transduced with MND.GRN lentiviral vector (+) were analyzed using Western blot with an antibody raised against human PGRN, showing release of human PGRN protein into the growth media by the transduced cells (FIG. 2).

FIG. 3 is a Western blot showing immortalized cell lines transduced with a lentiviral vector containing a transgene encoding human PGRN is N-linked glycosylated. Cell lysates were generated from human 239 T cell lines non-transduced (NT1, NT2, NT3, and NT4) or transduced with a lentiviral vector encoding human PGRN (MND.GRN-1, MND.GRN-2, MND.GRN-3, and MND.GRN-4) were generated in four independent rounds of transduction. Cell lysates were enzymatically digested with either EndoH (E.) or PNGase (P.) enzymes, or heated (H.) and analyzed using Western blot with an antibody raised against human progranulin. Enzymatic digestion by EndoH and PNGase indicate that the human PGRN protein produced by the transduced cells is N-linked glycosylated (FIG. 3).

### DEFINITIONS

As used herein, the terms “ablate,” “ablating,” “ablation,” and the like refer to the depletion of one or more cells in a population of cells in vivo or ex vivo. In some embodiments of the present disclosure, it may be desirable to ablate endogenous cells within a subject (e.g., a subject undergoing treatment for a disease described herein, such as a neurocognitive disorder (NCD; e.g., frontotemporal lobar degeneration (FTLD) or neuronal ceroid lipofuscinosis (NCL)) before administering a therapeutic population of cells to the subject. This can be beneficial, for example, in order to provide the newly-administered cells with an environment within which the cells may engraft. Ablation of a population of cells can be performed in a manner that selectively targets a specific cell type, for example, using antibody-drug conjugates that bind to an antigen expressed on the target cell and subsequently engender the killing of the target cell. Additionally or alternatively, ablation may be performed in a non-specific manner using cytotoxins that do not localize to a particular cell type but are instead capable of exerting their cytotoxic effects on a variety of different cells. Exemplary agents that may be used to ablate a population of endogenous cells in a subject, such as a population of endogenous microglia or microglial precursor cells in a subject undergoing therapy, e.g., for the treatment of an NCD, are busulfan, PLX3397, PLX647, PLX5622, treosulfan, clodronate liposomes, and combinations thereof. Examples of ablation include depletion of at least 5% of cells (e.g., at least 5%, 10%, 15%, 20%, 25%, 30%, 35%, 40%, 45%, 50%, or more) in a population of cells in vivo or in vitro. Quantifying cell counts within a sample of cells can be performed using a variety of cell-counting techniques, such as through the use of a counting chamber, a Coulter counter, flow cytometry, or other cell-counting methods known in the art.

As used herein, “administration” refers to providing or giving a subject a therapeutic agent (e.g., cells, such as pluripotent cells (e.g., embryonic stem cells (ESCs) or induced pluripotent stem cells (ISPCs)), multipotent cells (e.g., CD34+ cells such as, e.g., hematopoietic stem cells (HSCs) or myeloid precursor cells (MPCs)), blood lineage progenitor cells (BLPCS; e.g., monocytes), macrophages, microglial progenitor cells, or microglia that contain a transgene (e.g., a transgene capable of expression in macrophages or microglia) encoding a progranulin (PGRN) or a granulin (GRN), by any effective route. Exemplary routes of administration are described herein and below (e.g. intracerebroventricular (ICV) injection, intravenous (IV) injection, intrathecal (IT) injection, intraparenchymal (IP) injection, and stereotactic injection).

As used herein, “allogeneic” means cells, tissue, DNA, or factors taken or derived from a different subject of the same species. For example, in the context of transduced, PGRN-expressing or GRN-expressing cells that are administered to a subject for the treatment of an NCD, allogeneic cells may be cells that are obtained from a subject that is not the subject and are then transduced or transfected with a vector that directs the expression of the PGRN or the GRN. The phrase “directs expression” refers to the polynucleotide containing a sequence that encodes the molecule to be expressed. The polynucleotide may contain additional sequence that enhances expression of the molecule in question.

As used herein, “autologous” refers to cells, tissue, DNA, or factors taken or derived from an individual's own tissues, cells, or DNA. For example, in the context of transduced, PGRN-expressing or GRN-expressing cells that are administered to a subject for the treatment of an NCD (e.g., FTLD or NCL), the autologous cells may be cells obtained from the subject that are then transduced or transfected with a vector that directs the expression of PGRN or GRN.

As used herein, the term “ApoE” refers to apolipoprotein E, a member of a class of proteins involved in lipid transport. Apolipoprotein E is a fat-binding protein (apolipoprotein) that is part of the chylomicron and intermediate-density lipoprotein (IDLs). These are essential for the normal processing (catabolism) of triglyceride-rich lipoproteins. ApoE is encoded by the APOE gene. The term “ApoE” also refers to variants of the wild-type ApoE protein, such as proteins having at least 85% identity (e.g., at least 85%, 86%, 87%, 88%, 89%, 90%, 91%, 92%, 93%, 94%, 95%, 96%, 97%, 98%, 99%, or 99.9% identity, or more) to the amino acid sequence of wild-type ApoE, which is set forth in SEQ ID NO. 11.

As used herein, the term “blood lineage progenitor cell” or “BLPC” refers to any cell (e.g., a mammalian cell) capable of differentiating into one or more (e.g., 2, 3, 4, 5 or more) types of hematopoietic (i.e., blood) cells. A BLPC may differentiate into erythrocytes, leukocytes (e.g., such as granulocytes (e.g., basophils, eosinophils, neutrophils, and mast cells) or agranulocytes (e.g., lymphocytes and monocytes)), or thrombocytes. A BLPC may also include a differentiated blood cell (e.g., a monocyte) that can further differentiate into another blood cell type (e.g., a macrophage).

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15:8125-8148 (1989), incorporated herein by reference in its entirety. Multiple stop codons can be incorporated.

As used herein, the term “cognitive test” refers to a test that can be performed by a skilled practitioner in order to assess the cognitive capabilities of humans and other animals. A cognitive test may be used to assess inductive reasoning skills, intelligence quotient, cognitive development, memory, knowledge organization, metacognition, thought, mental chronometry. A cognitive test may be used to assess the performance of a subject across several cognitive domains, including, but not limited to executive function, learning and memory, language, perceptual-motor function, and social cognition. Examples of cognitive tests include, but are not limited to Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE). A skilled practitioner will recognize that other cognitive tests well-known in the art may also be used to assess cognitive function in a subject.

As used herein, the term “complex attention” refers to a cognitive function that describes a subject's (e.g., a human subject's) ability to maintain information in their mind for a short time and to perform an operation on that information (e.g., mental arithmetic). Impairment in complex attention may result in difficulty with focusing on conversations, difficulty filtering out unwanted information, problems with prospective memory (e.g., remembering to remember something later on), and inefficient memory for new information.

As used herein, the terms “condition” and “conditioning” refer to processes by which a subject is prepared for receipt of a transplant containing cells. Such procedures promote the engraftment of a cell transplant, for example, by selectively depleting endogenous microglia or hematopoietic stem cells, thereby creating a vacancy filled by an exogenous cell transplant. According to the methods described herein, a subject may be conditioned for cell transplant therapy by administration to the subject of one or more agents capable of ablating endogenous microglia and/or hematopoietic stem or progenitor cells (e.g., busulfan, treosulfan, PLX3397, PLX647, PLX5622, and clodronate liposomes), radiation therapy, or a combination thereof. Conditioning may be myeloablative or non-myeloablative. Other cell-ablating agents and methods well known in the art (e.g., antibody-drug conjugates) may also be used.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties of naturally occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the phrase “delirium or other mental disorder” refers to a condition such as delirium (i.e., a syndrome encompassing impaired attention, consciousness, and cognition that develops over a short period of time (e.g., hours to days)) or another disorder of the mind (e.g., schizophrenia, bipolar disorder, and major depression) that is distinct from a neurocognitive disorder and does not exhibit cognitive impairment as a core symptom. For example, a condition such as delirium or another mental disorder may differ from an NCD in that cognitive impairment may by a symptom that is associated with the disease but is not a central feature of said disease. Delirium or another mental disorder may differ from an NCD with respect to time to onset (e.g., hours to days in delirium versus months to years for an NCD), etiology (e.g., substance-induced delirium), symptom length (e.g., delirium may last hours to days whereas an NCD can last for years), and resolution (e.g., delirium may resolve completely, whereas an NCD does not resolve in most cases).

As used herein, the term “disrupt”, with respect to a gene, refers to preventing the formation of a functional gene product. A gene product is functional if it fulfills its normal (wild-type) functions. Disruption of the gene prevents expression of a functional factor encoded by the gene and contains an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods for genetically modifying cells so as to disrupt the expression of one or more genes are detailed in U.S. Pat. Nos. 8,518,701; 9,499,808; and US 2012/0222143, the disclosures of each of which are incorporated herein by reference in their entirety (in case of conflict, the instant specification is controlling).

As used herein, the terms “effective amount,” “therapeutically effective amount,” and a “sufficient amount” of composition, vector construct, viral vector, or cell described herein refer to a quantity sufficient to, when administered to the subject, including a mammal, for example a human, effect beneficial or desired results, including clinical results. As such, an “effective amount” or synonym thereof depends upon the context in which it is being applied. For example, in the context of treating an NCD (e.g., FTLD or NCL), it is an amount of the composition, vector construct, viral vector, or cell sufficient to achieve a treatment response as compared to the response obtained without administration of the composition, vector construct, viral vector or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, viral vector, or cell of the present disclosure is an amount which results in a beneficial or desired result in a subject as compared to a control. As defined herein, a therapeutically effective amount of a composition, vector construct, viral vector, or cell of the present disclosure may be readily determined by one of ordinary skill by routine methods known in the art. Dosage regime may be adjusted to provide the optimum therapeutic response.

As used herein, the terms “embryonic stem cell” and “ES cell” refer to an embryo-derived totipotent or pluripotent stem cell, derived from the inner cell mass of a blastocyst that can be maintained in an in vitro culture under suitable conditions. ES cells are capable of differentiating into cells of any of the three vertebrate germ layers, e.g., the endoderm, the ectoderm, or the mesoderm. ES cells are also characterized by their ability propagate indefinitely under suitable in vitro culture conditions. See, for example, Thomson et al., Science 282:1145 (1998).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “engraft” and “engraftment” refer to the process by which hematopoietic stem cells and progenitor cells, whether such cells are produced endogenously within the body or transplanted using any of the administration methods described herein (e.g. intravenous injection, intracerebroventricular injection, intraosseous injection, and/or bone marrow transplant), repopulate a tissue. The term encompasses all events surrounding or leading up to engraftment, such as tissue homing of cells and colonization of cells within the tissue of interest.

As used herein, the term “executive function” refers to a set of cognitive functions that facilitate cognitive control of behavior in a subject (e.g., a human). Executive function encompasses, e.g., selection and monitoring goal-directed behaviors, attentional control, cognitive inhibition, inhibitory control, working memory, and cognitive flexibility. An individual normally acquires or perfects executive functions across the lifespan, although this process may be derailed by the development of an NCD in the subject, which may adversely impact executive function.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a subject can manifest, for example, by detecting: an increase in the quantity or concentration of mRNA encoding a corresponding protein (as assessed, e.g., using RNA detection procedures described herein or known in the art, such as quantitative polymerase chain reaction (qPCR) and RNA seq techniques), an increase in the quantity or concentration of a corresponding protein (as assessed, e.g., using protein detection methods described herein or known in the art, such as enzyme-linked immunosorbent assays (ELISA), among others), and/or an increase in the activity of a corresponding protein (e.g., in the case of an enzyme, as assessed using an enzymatic activity assay described herein or known in the art) in a sample obtained from the subject.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “functional potential” as it pertains to a stem cell, such as a hematopoietic stem cell, refers to the functional properties of stem cells which include: 1) multi-potency (which refers to the ability to differentiate into multiple different blood lineages including, but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells); 2) self-renewal (which refers to the ability of stem cells to give rise to daughter cells that have equivalent potential as the mother cell, and further that this ability can repeatedly occur throughout the lifetime of an individual without exhaustion); and 3) the ability of stem cells or progeny thereof to be reintroduced into a transplant recipient whereupon they home to the stem cell niche and re-establish productive and sustained cell growth and differentiation.

As used herein, the term “furin-resistant IGF-II mutein” refers to an insulin-like growth factor II (IGF-II)-based peptide containing an altered amino acid sequence relative to wild-type IGF-II (SEQ ID NO. 12) that abolishes at least one native furin protease cleavage site or changes a sequence close or adjacent to a native furin protease cleavage site such that the furin cleavage is prevented, inhibited, reduced, or slowed down as compared to a wild-type human IGF-II peptide. As used herein, a furin-resistant IGF-II mutein is also referred to as an IGF-II mutein that is resistant to furin. Exemplary furin-resistant IGF-II muteins contain amino acid substitutions at positions corresponding to Arg37 and/or Arg40 of SEQ ID NO. 12.

As used herein, the term “furin protease cleavage site” (also referred to as “furin cleavage site” or “furin cleavage sequence”) refers to the amino acid sequence of a peptide or protein that serves as a recognition sequence for enzymatic protease cleavage by furin or furin-like proteases. Typically, a furin protease cleavage site has a consensus sequence Arg-X-X-Arg, where X is any amino acid. The cleavage site is positioned after the carboxy-terminal arginine (Arg) residue in the sequence. In some embodiments, a furin cleavage site has a consensus sequence Lys/Arg-X-X-X-Lys/Arg-Arg, where X is any amino acid. The cleavage site is positioned after the carboxy-terminal arginine (Arg) residue in the sequence.

As used herein, the term “furin” refers to any protease that can recognize and cleave the furin protease cleavage site as defined herein, including furin or furin-like protease. Furin is also known as paired basic amino acid cleaving enzyme (PACE). Furin belongs to the subtilisin-like proprotein convertase family. The gene encoding furin is known as FUR (FES Upstream Region).

As used herein, the term “glycosylation independent lysosomal targeting” or “GILT” refers to lysosomal targeting that is mannose-6-phosphate (M6P)-independent. A GILT tag may be used to target a protein (e.g., GBA) expressed as a GILT-tagged fusion protein (e.g., a GBA fusion protein coupled to an IGF-II mutein), to the lysosome.

As used interchangeably herein, the terms “cation-independent mannose-6-phosphate receptor (CI-MPR),” “M6P/IGF-II receptor,” “CI-MPR/IGF-II receptor,” “IGF-II receptor” or “IGF2 Receptor,” or abbreviations thereof, refer to the cellular receptor which binds both M6P and IGF-II.

As used herein, the terms “frontotemporal lobar degeneration” and “FTLD” refer to a complex clinical syndrome characterized by degeneration of brain tissue within the frontal and temporal lobes of the cerebral cortex. The terms “frontotemporal lobar degeneration” and “FTLD” may refer to any one of three clinically distinct variants of FTLD including: 1) behavioral-variant frontotemporal dementia (BVFTD), characterized by changes in behavior and personality, apathy, social withdrawal, perseverative behaviors, attentional deficits, disinhibition, and a pronounced degeneration of the frontal lobe. Additionally, BVFTD has a strong association with amyotrophic lateral sclerosis; 2) semantic dementia (SD) is characterized by fluent, anomic aphasia, progressive loss of semantic knowledge of words, objects, and concepts and a pronounced degeneration of the anterior temporal lobes. Furthermore, SD variant of FTLD exhibit a flat affect, social deficits, perseverative behaviors, and disinhibition; 3) progressive nonfluent aphasia (PNA) is characterized by motor deficits in speech production, reduced language expression, and pronounced degeneration of the perisylvian cortex. Histopathological profiles of FTLD patients generally fall into one of three broad phenotypes including those that exhibit aggregation and deposition of (i) microtubule-associated tau protein inclusions; (ii) tau-negative, ubiquitin and TAR DNA-binding protein 43 (TDP-43)-positive protein inclusions, or (iii) ubiquitin and fused in sarcoma (FUS)-positive protein inclusions. For a comprehensive description of the clinical presentation and histopathology of FTLD, see Rabinovici and Miller, CNS Drugs 24:375-398 (2010), the disclosure of which is herein incorporated by reference in its entirety.

As used herein, the term “general population” refers to an entire population of individuals having a particular characteristic of interest (e.g., age, medical history, education, socioeconomic status, or lifestyle, among others). Alternatively, the term “general population” may refer to a subset of the entire population of individuals having a particular characteristic of interest, such as, e.g., a random sample having a defined sample size. According to the methods disclosed herein, the general population may serve as a practical referent (e.g., a reference population) to which a measured variable can be compared. For example, a subject diagnosed with an NCD may have their cognition assessed using a cognitive test disclosed herein and the score obtained by the subject on the test may be compared against performance of individuals in the general population (e.g., the entire general population or a random sample of the general population) on the same test. The size of the random sample of the general population may be determined by a skilled practitioner using methods well-known in the art. For example, a skilled practitioner may perform a power analysis prior to collecting data (e.g., prior to conducting a cognitive test on a subject) to determine the smallest sample that is needed to detect a statistically significant effect with a desired level of confidence.

As used herein, the terms “granulin” and “GRN” refer to the peptide products resulting from cleavage of the precursor protein PGRN. GRN peptides are involved in a variety of biological functions including development, immunity, cell survival and proliferation, and tumorigenesis. Full-length wild-type human PGRN peptide has 7.5 GRN domains (e.g., 7 GRN domains, each approximately 60 amino acids in length, and a 30 amino acid paragranulin (para-GRN) domain, that can be individually cleaved by proteases. The terms “granulin” and “GRN” also refer to variants of wild-type human granulin peptides and nucleic acids encoding the same, such as variant proteins having at least 85% sequence identity (e.g., at least 85%, 86%, 87%, 88%, 89%, 90%, 91%, 92%, 93%, 94%, 95%, 96%, 97%, 98%, 99%, or 99.9% identity, or more) to any of the amino acid sequences of a wild-type GRN peptides (e.g., any one of SEQ ID NOs. 2-9), provided that the GRN variant encoded retains the therapeutic function of the wild-type GRN. The terms “granulin” and “GRN” may also refer to a GRN protein in which the natural secretory signal peptide is present. Additionally, the terms “granulin” and “GRN” may refer to a “GRN fusion protein,” which is a protein in which the GRN is operably linked to another polypeptide, half-life-modifying agent, or therapeutic agent, such as an ApoE receptor-binding (Rb) domain (such as a Rb domain having the amino acid sequence of residues 25-185, 50-180, 75-175, 100-170, 125-160, or 130-150 of SEQ ID NO. 11). As used herein, the term “GRN” may refer to the peptide or the gene encoding this protein, depending upon the context, as will be appreciated by one of skill in the art.

As used herein, the terms “hematopoietic stem cells” and “HSCs” refer to immature blood cells having the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSCs are CD34-. In addition, HSCs also refer to long term repopulating HSC (LT-HSC) and short-term repopulating HSC (ST-HSC). LT-HSC and ST-HSC are differentiated, based on functional potential and on cell surface marker expression. For example, human HSC are a CD34+, CD38−, CD45RA−, CD90+, CD49F+, and lin− (negative for mature lineage markers including CO2, CD3, CD4, CD7, CD8, CD10, CD11B, CD19, CD20, CD56, CD235A). In mice, bone marrow LT-HSC are CD34−, SCA-1+, C-kit+, CD135−, Slamf1/CD150+, CD48−, and lin− (negative for mature lineage markers including Ter119, CD11b, Gr1, CD3, CD4, CD8, B220, IL-7ra), whereas ST-HS Care CD34+, SCA-1+, C-kit+, CD135−, Slamf1/CD150+, and lin− (negative for mature lineage markers including Ter119, CD11b, Gr1, CD3, CD4, CD8, B220, IL-7ra). In addition, ST-HSC are less quiescent (i.e., more active) and more proliferative than LT-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSCs can be used in any of the methods described herein. Optionally, ST-HSCs are useful because they are highly proliferative and thus, can more quickly give rise to differentiated progeny.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign and are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the phrase “independence or normal daily functioning” refers to the ability of a subject (e.g., a human) to successfully perform everyday activities without assistance from a caretaker or a social worker. Non-limiting examples of activities that enable an individual to independently carry out daily functions include, e.g., social, occupational, or academic functioning, personal hygiene, grooming, dressing, toilet hygiene, functional mobility (e.g., ability to walk, get in and out of bed), and self-feeding. A subject diagnosed with a major NCD, may have difficulty independently performing normal daily functions, whereas a subject diagnosed with mild NCD may not have difficulty independently performing daily tasks.

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-pluripotent cell that can include, for example, Oct3/4, Sox family transcription factors (e.g., Sox1, Sox2, Sox3, Soxl5), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. See, for example, Takahashi and Yamanaka, Cell 126:663 (2006).

As used herein, the term “IRES” refers to an internal ribosomal entry site. In general, an IRES sequence is a feature that allows eukaryotic ribosomes to bind an mRNA transcript and begin translation without binding to a 5′ capped end. An mRNA containing an IRES sequence produces two translation products, one initiating form the 5′ end of the mRNA and the other from an internal translation mechanism mediated by the IRES.

As used herein, the term “language” refers to a cognitive ability of a subject to learn and use systems of complex communication, or to describe the rules that govern these systems, or the collection of utterances that may be generated from such rules. Language ability may be impaired in a subject with an NCD if the subject exhibits, e.g., limited vocabulary, inability to produce complex grammar, frequent lexical errors, or aphasia, among others.

As used herein, the phrase “learning and memory” refer to a cognitive ability that encompasses the acquisition of skills or knowledge and expression of acquired skills or knowledge (e.g., learning to say a new word and uttering the new word, respectively). “Learning and memory” may refer to two independent processes of 1) acquiring new skills or knowledge (i.e., learning); and 2) processing, storing, and recalling the learned skill or knowledge (i.e., memory), which may differ by timescales (learning is generally slower and more effortful than recalling a memory or performing a learned skill) and neurobiological basis. A subject diagnosed with an NCD may have impaired learning and memory relative to a healthy subject.

As used herein, the term “lysosomal disease” refers to a large set of about 50 genetic metabolic disorders that are caused by abnormal lysosomal function. Defects in genes involved in metabolism of lipids (e.g., the progranulin gene), glycoproteins, or mucopolysaccharides are common causes for lysosomal disease. Accumulation of these molecules in the cell eventually leads to cell death. Common symptoms of lysosomal disease are highly variable and dependent on the particular disease, but may include developmental delay, movement disorders, seizures, dementia, hearing and/or vision impairment, enlarged liver or spleen, pulmonary and cardiac problems, and abnormal bone development. Non-limiting examples of lysosomal disease include neuronal ceroid lipofuscinosis, sphingolipidoses, galactosialidosis, gangliosidoses, Farber disease, Krabbe disease, Gaucher disease, lysosomal acid lipase deficiency, Niemann-Pick disease, sulfatidosis, mucopolysaccharidoses, mucolipidosis, lipodoses, alpha-mannosidosis, beta-mannosidosis, aspartylglucosaminuria, fucosidosis, lysosomal transport diseases, glycogen storage diseases, and cholesteryl ester storage disease.

As used herein, the term “macrophage” refers to a type of white blood cell that engulfs and digests cellular debris, foreign substances, microbes, cancer cells, and anything else that does not have 15 the types of proteins specific to healthy body cells on its surface in a process called phagocytosis. Macrophages are found in essentially all tissues, where they patrol for potential pathogens by amoeboid movement. They take various forms (with various names) throughout the body (e.g., histiocytes, Kupffer cells, alveolar macrophages, microglia, and others), but all are part of the mononuclear phagocyte system. Besides phagocytosis, they play a critical role in non-specific defense (innate immunity) and also 20 help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines.

As used herein, the terms “microglia” or “microglial cell” refer to a type of neuroglial cell found in the brain and spinal cord that function as resident macrophage cells and the principal line of immune defense in the central nervous system. Primary functions of microglial cells include immune surveillance, phagocytosis, extracellular signaling (e.g., production and release of cytokines, chemokines, prostaglandins, and reactive oxygen species), antigen presentation, and promotion of tissue repair and regeneration.

As used herein, the term “microglial progenitor cell” refers to a precursor cell that gives rise to microglial cells. Microglial precursor cells originate in the yolk sac during a limited period of embryonic development, infiltrate the brain mesenchyme, and perpetually renew themselves throughout life.

As used herein, the term “miRNA targeting sequence” refers to a nucleotide sequence located in the 3′-UTR of a target mRNA molecule which is complementary to a specific miRNA molecule (e.g. miR-126) such that they may hybridize and promote RNA-induced silencing complex-dependent and Dicer-dependent mRNA destabilization and/or cleavage, thereby preventing the expression of an mRNA transcript.

As used herein, the term “monocyte” refers to a type of white blood cell (i.e., a leukocyte) that is capable of differentiating into macrophages and myeloid lineage dendritic cells. Monocytes constitute an important component of the vertebrate adaptive immune response. Three different types of monocytes are known to exist, including classical monocytes characterized by strong expression of the CD14 cell surface receptor and no CD16 expression (i.e., CD14++CD16-), non-classical monocytes exhibiting low levels of CD14 expression and co-expression of C16 (CD14+CD16++), and intermediate monocytes exhibiting high levels of CD14 expression and low levels of C16 expression (CD14++CD16+). Monocytes perform a variety of functions that serve the immune system, including phagocytosis, antigen presentation, and cytokine secretion.

As used herein, the term “multipotent cell” refers to a cell that possesses the ability to develop into multiple (e.g., 2, 3, 4, 5, or more) but not all differentiated cell types. Non-limiting examples of multipotent cells include cells of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of multipotent cells are CD34+ cells.

As used herein, the term “mutation” refers to a change in the nucleotide sequence of a gene. Mutations in a gene may occur naturally as a result of, for example, errors in DNA replication, DNA repair, irradiation, and exposure to carcinogens or mutations may be induced as a result of administration of a transgene expressing a mutant gene. Examples of mutations may include frameshift, nonsense, missense, insertion, deletion, and transversion mutations. Frameshift mutations may refer to a change in the nucleotide sequence such that the position of the ribosomal reading frame on the mRNA is shifted, resulting in inappropriate translation of the RNA. Nonsense mutations may refer to a change in a single nucleotide of a gene that results in a premature stop codon within the transcript. The premature stop codon may result in the translation of a truncated protein product or in nonsense-mediated decay of the transcript. Missense mutations may refer to a single nucleotide change within the gene that results in a codon that codes for a different amino acid, which may alter the physicochemical properties of the protein product and/or render it nonfunctional. Insertion mutations may refer to the introduction of one or more nucleotide into the coding region of a gene, which can result in a frameshift and typically the generation of a premature stop codon. Deletion mutations may refer to the removal of one or more nucleotides from the DNA sequence of a gene that can result in a frameshift and commonly a premature stop codon. Transversion mutations may refer to the change of a single purine nucleotide (e.g. adenine, guanine) to a single pyrimidine nucleotide (e.g. cytosine, thymine). Transversion mutations may result in no change to the translated protein product (e.g. silent mutations) or may change the amino acid identity within a single codon, thereby altering the physicochemical properties and function of the translated protein product. The nomenclature for describing mutations and sequence variations uses the format “reference sequence.code,” where the reference sequence may be “c” designating coding DNA, “g” designating genomic DNA, “m” designating mitochondrial DNA, “r” designating RNA, or “p” designating protein and the code may contain symbols including “>” designating substitution, “_” designating range, “;” designating more change in one allele, “,” designating more transcripts/mosaicisms, “( )” designating uncertain change, “[ ]” designating allele, “del” designating deletion, “dup” designating duplication, “ins” designating insertion, “inv” designating inversion, “cony” designating conversion, “ext” designating extension, “X” designating a stop codon, “fsX” designating a frameshift resulting in a stop codon, “o” designating opposite strand, and “t” designating translocation. For example, a p.T382NfsX32 mutation in the PGRN gene corresponds to a change in the protein at amino acid 382 where a threonine is substituted for asparagine as a result of a frameshift mutation, where the length of the frameshift is 32 nucleotide base pairs including the stop codon.

As used herein, the term “myeloablative” or “myeloablation” refers to a conditioning regiment that substantially impairs or destroys the hematopoietic system, typically by exposure to a cytotoxic agent (e.g., busulfan) or radiation. Myeloablation encompasses complete myeloablation brought on by high doses of cytotoxic agent or total body irradiation that destroys the hematopoietic system.

As used herein, the terms “neurocognitive disorder” or “NCD” refer to a set of clinical disorders or syndromes in which the primary clinical deficit is cognitive function, such as a deficit in, e.g., complex attention, executive function, learning and memory, language, perceptual-motor function, and social cognition. NCD is characterized as an acquired condition, rather than a developmental one. For example, an NCD is a condition in which disrupted cognition was not evident since birth or very early life, therefore requiring that cognitive function in NCD declined from a previously acquired level. NCD is distinguished from other disorders in which patients present with cognitive impairment in that NCD includes only disorders in which the core deficits are cognitive. NCD may be “major NCD” or “mild NCD.” Major NCD is characterized by significant cognitive decline that interferes with personal independence and normal daily functioning and is not due to delirium or other mental disorder. Mild NCD is characterized by moderate cognitive decline that does not interfere with personal independence and normal daily functioning and is not due to delirium or other mental disorder. Major and mild NCD may also be differentiated on the basis of quantitative cognitive testing across any one of the specific cognitive functions described above. For example, major NCD can be characterized by a score obtained on a cognitive test by a subject identified as having or at risk of developing NCD that is more than two standard deviations away from the mean score of a reference population (e.g., the mean score of a general population) or a score that is in the third percentile of the distribution of scores of the reference population. Mild NCD can be characterized by a score obtained on a cognitive test by a subject identified as having or at risk of developing NCD that is between one to two standard deviations away from the mean score of a reference population (e.g., the mean score of a general population) or a score that is between the 3^{rd} and 16^{th} percentile of the distribution of scores of the reference population. Non-limiting examples of cognitive tests that can be used to categorize an NCD patient as having either major or mild NCD include ADB, AWV, GPCOG, HRA, MIS, MMSE, MoCA, SLUMS, and Short IQCODE. Furthermore, NCD (e.g., major or mild NCD) includes syndrome subtypes that designate the particular etiological origin of the NCD, such as, e.g., frontotemporal lobar degeneration (FTLD) or a lysosomal disease (e.g., neuronal ceroid lipofuscinosis (NCL)). As used herein, the terms “frontotemporal NCD” and “NCD due to a lysosomal disease” correspond to NCD caused by frontotemporal lobar degeneration and a lysosomal disease (e.g., NCL), respectively.

As used herein, the terms “neuronal ceroid lipofuscinosis” and “NCL” refer to a collection of at least eight clinically recognized lysosomal storage disorders that are caused by the accumulations of lipofuscin within cells of the body, such as neuronal, liver, spleen, myocardium, and kidney cells. NCL clinically presents with profound neurodegeneration and progressive and irreversible loss of motor and cognitive abilities, although the disease severity and clinical presentation may depend on the particular NCL variant. Known variants of NCL include the infantile variant, also known as Santovuori-Haltia disease (SHD), the late infantile variant known as Jansky-Bielschowsky disease (JBD), the Finnish late infantile variant (FLI), the variant late infantile (VLI), the CLN7 variant (CLN7), the CLN8 variant (CLN8), the Turkish late infantile variant (TLI), the type 9 variant (T9), the CLN10 variant (CLN10), the juvenile variant also known as Batten disease (BD), and the adult variant also known as Kuf's disease (KD). SHD is associated with early visual loss that progressively turns to complete retinal blindness by the age of 2, followed by a vegetative state at 3 years, and brain death by year 4. This variant is also associated with the spontaneous occurrence of epileptic seizures. The JBD variant emerges between ages 2 to 4 and is associated with ataxia, epileptic seizures, progressive cognitive decline, and abnormal speech development and typically results in death by age 8. BD typically emerges between 4 and 10 years of age and include symptoms such as vision loss, epileptic seizures, cognitive dysfunction, and premature death. NCL patients having the KD variant generally present with milder symptoms than SHD and BD variants and have a life expectancy of around 40 years. For a comprehensive description of NCL, see Mink et al., Journal of Child Neurology 28:1101-5 (2013), Nita et al., Epileptic Disorders 18:73-88 (2016), and Mole & Cotman, Biochimica et Biophysica Acta 1852:2237-41 (2015), the disclosures of which are herein incorporated by reference in their entirety.

As used herein, the term “non-myeloablative” or “myelosuppressive” refers to a conditioning regiment that does not eliminate substantially all hematopoietic cells of host origin.

As used herein, the term “pluripotent cell” refers to a cell that possesses the ability to develop into more than one differentiated cell type, such as a cell type of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of cells are ESCs and iPSCs.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the terms “progranulin” and “PGRN” refer to the secreted trophic factor and precursor peptide for granulin. The gene is located on chromosome 17q21.31 and is also known as granulin precursor, proepithelin, PEPI, PC cell-derived growth factor, granulin-epithelin, CLN11, PCDFGF, GP88, GEP, granulins, acrogranin. The terms “progranulin” and “PGRN” also refer to variants of wild-type human PGRN peptides and nucleic acids encoding the same, such as variant proteins having at least 85% sequence identity (e.g., at least 85%, 86%, 87%, 88%, 89%, 90%, 91%, 92%, 93%, 94%, 95%, 96%, 97%, 98%, 99%, or 99.9% identity, or more) to the amino acid sequence of the wild-type PGRN peptide (e.g., SEQ ID NO. 1) or polynucleotides having at least 85% sequence identity (e.g., at least 85%, 86%, 87%, 88%, 89%, 90%, 91%, 92%, 93%, 94%, 95%, 96%, 97%, 98%, 99%, or 99.9% identity, or more) to the nucleic acid sequence of the wild-type PGRN gene (e.g., SEQ ID NO. 2), provided that the PGRN variant encoded retains the therapeutic function of the wild-type PGRN. The terms “progranulin” and “PGRN” may also refer to variants of PGRN having 2 or more (e.g., 2, 3, 4, 5, 6, 7, 8, or more) granulin (GRN) domains having the amino acid sequences of any one of SEQ ID NOs. 2-9. The terms “progranulin” and “PGRN” may also refer to variants of PGRN having from 2 to 16 (e.g., 2, 3, 4, 5, 6, 7, 8, 9, 10, 11, 12, 13, 14, 15, 16) GRN domains having the amino acid sequences of any one of SEQ ID NOs. 2-9. The terms “progranulin” and “PGRN” may also refer to a PGRN protein in which the natural secretory signal peptide is present. Additionally, the terms “progranulin” and “PGRN” may refer to a “PGRN fusion protein,” which is a protein in which the PGRN is operably linked to another polypeptide, half-life-modifying agent, or therapeutic agent, such as an ApoE receptor-binding (Rb) domain (such as a Rb domain having the amino acid sequence of residues 25-185, 50-180, 75-175, 100-170, 125-160, or 130-150 of SEQ ID NO. 11). As used herein, the term “PGRN” may refer to the peptide or the gene encoding this protein, depending upon the context, as will be appreciated by one of skill in the art.

As used herein, patients suffering from “progranulin-associated FTLD or NCL” and “PGRN-associated FTLD or NCL” are those patients that have been diagnosed as having FTLD or NCL and also contain a deleterious mutation in the PGRN gene. Over 70 pathogenic mutations have been reported in the PGRN gene, the majority of which result in a premature stop codon and nonsense-mediated decay of truncated PGRN mRNA. PGRN mutations are described in Gijselinck et al., Human Mutation 29:1373-1386 (2012) and Pottier et al., Journal of Neurochemistry. 138:32-53 (2016), the disclosures of which are incorporated herein by reference as they pertain to human PGRN mutations.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, a potent “receptor-binding peptide (Rb) derived from ApoE” has the ability to translocate proteins across the BBB into the brain when engineered as fusion proteins. This method can therefore function to selectively open the BBB for therapeutic agents (e.g., soluble PGRN or GRN) when engineered as a fusion protein. This peptide can be readily attached to diagnostic or therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Exemplary Rb domains can be found in the N-terminus of ApoE. For example, Rb domains useful in conjunction with the compositions and methods described herein are polypeptides having the amino acid sequence of residues 1 to 191 of SEQ ID NO. 11, residues 25 to 185 of SEQ ID NO. 11, residues 50 to 180 of SEQ ID NO. 11, residues 75 to 175 of SEQ ID NO. 11, residues 100 to 170 of SEQ ID NO. 11, or residues 125 to 165 of SEQ ID NO. 11, as well as variants thereof, such as polypeptides having at least 85% sequence identity (e.g., at least 85%, 86%, 87%, 88%, 89%, 90%, 91%, 92%, 93%, 94%, 95%, 96%, 97%, 98%, 99%, or greater, sequence identity) with respect to any of these sequences. An exemplary Rb domain is the region of ApoE having the amino acid sequence of residues 159 to 167 of SEQ ID NO. 11.

As used herein, the term “regulatory sequence” includes promoters, enhancers and other expression control elements (e.g., polyadenylation signals) that control the transcription or translation of the antibody chain genes. Such regulatory sequences are described, for example, in Perdew et al., Regulation of Gene Expression (Humana Press, New York, N.Y., (2014)); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the term “secretory signal peptide” refers to a short (usually between 16-60 amino acids) peptide region within the precursor protein that directs secretion of the precursor protein from the cytoplasm of the host into the periplasmic space or into the extracellular space. Such secretory signal peptides are generally located at the amino terminus of the precursor protein. In some embodiments, the secretory signal peptide is linked to the amino terminus. Typically, secretory signal peptides are cleaved during transit through the cellular secretion pathway. Cleavage is not essential as long as the secreted protein retains its desired activity. Exemplary secretory signal peptide includes the PGRN secretory signal peptide.

As used herein, the term “social cognition” refers to a cognitive function that encompasses a set of skills that govern how subjects (e.g., humans) process, store, and apply information about other conspecific subjects (e.g., other humans) and social situations. Non-limiting examples of social cognition include, e.g., emotional responses to social stimuli, performance on theory of mind tasks, ability to recognize faces, impulse control in social contexts, and joint attention. A subject diagnosed with an NCD may exhibit impaired social cognition relative to a healthy subject.

As used herein, the terms “stem cell” and “undifferentiated cell” refer to a cell in an undifferentiated or partially differentiated state that has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the term “transgene” refers to a recombinant nucleic acid (e.g., DNA or cDNA) encoding a gene product (e.g., PGRN or GRN). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s) and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “subject” and “patient” refer to an animal (e.g., a mammal, such as a human). A subject to be treated according to the methods described herein may be one who has been diagnosed with an NCD, or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a viral vector construct or a part thereof into a cell, and subsequent expression of a transgene encoded by the vector construct or part thereof in the cell.

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of the PGRN or the GRN as described herein include plasmids that contain regulatory sequences, such as promoter and enhancer regions, which direct gene transcription. Other useful vectors for expression of the PGRN or the GRN contain polynucleotide sequences that enhance the rate of translation of these genes or improve the stability or nuclear export of the mRNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

### DETAILED DESCRIPTION

Described herein are compositions and methods for the treatment of a neurocognitive disorder (NCD), such as, e.g., frontotemporal lobar degeneration (FTLD) or neuronal ceroid lipofuscinosis (NCL), in a subject (such as a mammalian subject, for example, a human). Using the compositions and methods described herein, one can treat an NCD (e.g., FTLD or NCL (e.g., progranulin (PGRN)-associated FTLD or NCL)) in a subject (e.g., a human subject) by administering cells, such as pluripotent cells, embryonic stem cells (ESCs), induced pluripotent stem cells (iPSCs), multipotent cells, CD34+ cells, hematopoietic stem cells (HSCs), myeloid progenitor cells (MPCs), blood line progenitor cells (BLPCs), monocytes, macrophages, microglial progenitor cells, or microglia containing a transgene (e.g., a transgene capable of expression in a macrophage or a microglial cell) encoding a PGRN or a granulin (GRN). For example, described herein are compositions containing cells that have been modified ex-vivo to express the PGRN or the GRN. The sections that follow describe the compositions and methods useful for the treatment of an NCD in further detail.

### Neurocognitive Disorders

Neurocognitive disorders (NCDs) are defined as a collection of disorders that feature cognitive impairment as a core symptom and that show cognitive decline relative to a previously higher level of cognition (e.g., acquired impairment), rather than a developmental impairment. NCDs are broadly divided into major or mild syndromes (e.g., major NCD and mild NCD) based on the degree of impairment diagnosed in the subject. Furthermore, NCDs can be categorized on the basis of their etiological origin. For example, non-limiting examples of NCD may include frontotemporal NCD, NCD due to a lysosomal disease (e.g., NCL), NCD due to AD, vascular NCD, NCD with Lewy bodies, NCD due to Parkinson disease, frontotemporal NCD, NCD due to traumatic brain injury, NCD due to HIV infection, substance/medication-induced NCD, NCD due to Huntington's disease, NCD due to prion disease, NCD due to another medical condition, NCD due to multiple etiologies, and unspecified NCD. The compositions and methods disclosed herein are useful for the treatment of NCDs (e.g., FTLD or NCL).

### Frontotemporal Lobar Degeneration

FTLD is a clinical syndrome characterized by progressive neurodegeneration in the frontal and temporal lobes of the cerebral cortex. The manifestation of FTLD is complex and heterogeneous, and may present as one of three clinically distinct variants including: 1) behavioral-variant frontotemporal dementia (BVFTD), characterized by changes in behavior and personality, apathy, social withdrawal, perseverative behaviors, attentional deficits, disinhibition, and a pronounced degeneration of the frontal lobe; 2) semantic dementia (SD), characterized by fluent, anomic aphasia, progressive loss of semantic knowledge of words, objects, and concepts and a pronounced degeneration of the anterior temporal lobes. Furthermore, SD variant of FTLD exhibit a flat affect, social deficits, perseverative behaviors, and disinhibition; or 3) progressive nonfluent aphasia (PNA); characterized by motor deficits in speech production, reduced language expression, and pronounced degeneration of the perisylvian cortex. Neuronal loss in brains of FTLD patients is associated with one of three distinct neuropathologies: 1) the presence of tau-positive neuronal and glial inclusions; 2) ubiquitin (ub)-positive and TAR DNA-binding protein 43 (TDP43)-positive, but tau-negative inclusions; or 3) ub and fused in sarcoma (FUS)-positive, but tau and TDP-43-negative inclusions. These neuropathologies are considered to be important in the etiology of FTLD.

Nearly half of FTLD patients have a first-degree family member with dementia, ALS, or Parkinson's disease, suggesting a strong genetic link to the cause of the disease. A number of mutations in chromosome 17q21 have been linked to FTLD presentation.

### Neuronal Ceroid Lipofuscinosis

NCL is an umbrella term that relates to a collection of at least eight clinically recognized lysosomal storage disorders caused by the accumulations of lipofuscin within cells of the body, such as neuronal, liver, spleen, myocardium, and kidney cells. NCL clinically presents with profound neurodegeneration and progressive and irreversible loss of motor and cognitive abilities, although the disease severity and clinical presentation may depend on the particular NCL variant.

Known variants of NCL include the infantile variant, also known as Santovuori-Haltia disease (SHD), the late infantile variant known as Jansky-Bielschowsky disease (JBD), the Finnish late infantile variant (FLI), the variant late infantile (VLI), the CLN7 variant (CLN7), the CLN8 variant (CLN8), the Turkish late infantile variant (TLI), the type 9 variant (T9), the CLN10 variant (CLN10), the juvenile variant also known as Batten disease (BD), and the adult variant also known as Kuf's disease (KD). SHD is associated with early visual loss that progressively turns to complete retinal blindness by the age of 2, followed by a vegetative state at 3 years, and brain death by year 4. This variant is also associated with the spontaneous occurrence of epileptic seizures. The JBD variant emerges between ages 2 to 4 and is associated with ataxia, epileptic seizures, progressive cognitive decline, and abnormal speech development and typically results in death by age 8. BD typically emerges between 4 and 10 years of age and include symptoms such as vision loss, epileptic seizures, cognitive dysfunction, and premature death. NCL patients having the KD variant generally present with milder symptoms than SHD and BD variants and have a life expectancy of around 40 years.

### Progranulin-Associated Frontotemporal Dementia and Neuronal Ceroid Lipofuscinosis

Studies investigating the link between chromosome 17q21 and FTLD have found a number of FTLD-related mutations in the PGRN gene. These mutations often result in aggregation and accumulation of ub-positive, TDP43-positive, tau-negative neuropathological inclusions in brains of FTLD patients. PGRN is a secreted precursor peptide to a number of mature GRN proteins and is thought to function primarily as a neurotrophic growth factor, promoting neuronal differentiation and survival. PGRN has also been demonstrated to serve anti-inflammatory and neuroprotective functions. PGRN is expressed ubiquitously, but as a result of its association with FTLD, significant attention has been directed to the central nervous system (CNS) where it is expressed in multiple cell types including neuronal, glial, and endothelial cells. Over 70 loss-of-function mutations in the PGRN gene have been identified in FTLD, the vast majority of which result in haploinsufficiency and a reduction in serum PGRN levels by more than a 50%. PGRN mutations are described in Gijselinck et al., Human Mutation 29:1373-86 (2008), the disclosure of which is incorporated herein by reference as they relate to human PGRN mutations. The effects of PGRN mutations are dose dependent as homozygous patients completely lacking functional PGRN protein develop NCL, suggesting an additional role for this protein in normal lysosomal function. For a comprehensive description of NCL and related genetic mutations, see Mink et al. Journal of Child Neurology 28:1101-5 (2013), Nita et al. Epileptic Disorders 18:73-88 (2016), and Mole & Cotman, Biochimica et Biophysica Acta 1852:2237-41 (2015), and Ward et al., Science Translational Medicine 9(385):eaah5642, the disclosures of which are herein incorporated by reference in their entirety.

Clinical management of FTLD has primarily employed selective serotonin reuptake inhibitors (SSRIs) and antipsychotics to manage the changes in affect and behavior that accompany FTLD. Similarly, the majority of interventional treatments targeted at NCL pathology aim to reduce the severity of disease symptoms, such as epileptic seizures, motor deficits, enhanced immune response, and pain management. This strategy, however, is targeted at ameliorating the symptoms of the disease without addressing its development and progression. Unlike these treatments, the compositions and methods described herein provide the benefit of treating a different biochemical phenomenon that can underlie the development of an NCD such as FTLD or NCL. As such, the compositions and methods described herein target the physiological cause of the disease, representing a potential curative therapy.

The compositions and methods described herein can be used to treat an NCD by administering cells (e.g., pluripotent cells, ESCs, iPSCs, multipotent cells, CD34+ cells, HSCs, MPCs, BLPCs, monocytes, macrophages, microglial progenitor cells, or microglia) containing a transgene (such as a transgene capable of expression in macrophages or microglial cells) encoding PGRN or GRN. These compositions and methods can be used to treat an NCD with any etiology, e.g., genetic mutation, environmental toxin, or sporadic. These compositions and methods can also be used to treat subjects with PGRN-associated FTLD or NCL. The compositions and methods described herein can be used to treat subjects with normal PGRN or GRN activity, reduced PGRN or GRN activity, and subjects whose PGRN mutational status and/or PGRN or GRN activity level is unknown. The compositions and methods described herein may also be administered as a preventative treatment to subjects at risk of developing an NCD, e.g., subjects with a PGRN mutation, subjects with reduced PGRN or GRN activity, and subjects with a mutation in one or more of the genes associated with an NCD (e.g., FTLD or NCL).

### Progranulin and Granulin Constructs

Transgene-containing constructs that may be used in conjunction with the compositions and methods described herein include polynucleotides that encode the wild-type human PGRN (the amino acid sequence of which is shown as SEQ ID NO. 1, below), or a variant thereof, such as a polynucleotide that encodes a protein having at least 85% sequence identity (e.g., at least 85%, 90%, 95%, 96%, 97%, 98%, 99%, or more, sequence identity) to the amino acid sequence of SEQ ID NO. 1. In some embodiments, the PGRN comprises at least 2 GRN domains (e.g., at least 2, 3, 4, 5, 6, 7, 8, or more GRN domains) having the amino acid sequence of any one of SEQ ID NOs. 2-9. In some embodiments, the PGRN comprises from 2 to 16 GRN domains (e.g., 2, 3, 4, 5, 6, 7, 8, 9, 10, 11, 12, 13, 14, 15, or 16 GRN domains).

In some embodiments, the polynucleotide encoding the wild-type PGRN or GRN may be a codon-optimized polynucleotide to confer resistance against degradation by nucleases and inhibitory RNAs directed to the endogenous PGRN or GRN, as described in detail below. In some embodiments, the codon-optimized transgene encoding the PGRN or the GRN contains a polynucleotide having at least 85% sequence identity (e.g., at least 85%, 90%, 95%, 96%, 97%, 98%, 99%, or more, sequence identity) to the nucleic acid sequence of SEQ ID NO. 19.

The wild-type human PGRN protein has the amino acid sequence of (Gen Bank accession number: NP_002078.1):

(SEQ ID NO. 1)
MWTLVSWVALTAGLVAGTRCPDGQFCPVACCLDPGG
ASYSCCRPLLDKWPTTLSRHLGGPCQVDAHCSAGH
SCIFTVSGTSSCCPFPEAVACGDGHHCCPRGFHCS
ADGRSCFQRSGNNSVGAIQCPDSQFECPDFSTCCV
MVDGSWGCCPMPQASCCEDRVHCCPHGAFCDLVHT
RCITPTGTHPLAKKLPAQRTNRAVALSSSVMCPDA
RSRCPDGSTCCELPSGKYGCCPMPNATCCSDHLHC
CPQDTVCDLIQSKCLSKENATTDLLTKLPAHTVGD
VKCDMEVSCPDGYTCCRLQSGAWGCCPFTQAVCCE
DHIHCCPAGFTCDTQKGTCEQGPHQVPWMEKAPAH
LSLPDPQALKRDVPCDNVSSCPSSDTCCQLTSGEW
GCCPIPEAVCCSDHQHCCPQGYTCVAEGQCQRGSE
IVAGLEKMPARRASLSHPRDIGCDQHTSCPVGQTC
CPSLGGSWACCQLPHAVCCEDRQHCCPAGYTCNVK
ARSCEKEVVSAQPATFLARSPHVGVKDVECGEGHF
CHDNQTCCRDNRQGWACCPYRQGVCCADRRHCCPA
GFRCAARGTKCLRREAPRWDAPLRDPALRQLL

The wild-type human paragranulin (para-GRN) has the amino acid sequence of:

(SEQ ID NO. 2)
TRCPDGQFCPVACCLDPGGASYSCCRPLLD

The wild-type human granulin-1 (GRN-1) peptide has the amino acid sequence of:

(SEQ ID NO. 3)
GGPCQVDAHCSAGHSCIFTVSGTSSCCPFPEAVACGD
GHHCCPRGFHCSADGRSCF

The wild-type human granulin-2 (GRN-2) peptide has the amino acid sequence of:

(SEQ ID NO. 4)
AIQCPDSQFECPDFSTCCVMVDGSWGCCPMPQASCCE
DRVHCCPHGAFCDLVHTRCI

The wild-type human granulin-3 (GRN-3) peptide has the amino acid sequence of:

(SEQ ID NO. 5)
VMCPDARSRCPDGSTCCELPSGKYGCCPMPNATCCSDHLHCCPQDTVCDLI
QSKCL

The wild-type human granulin-4 (GRN-4) peptide has the amino acid sequence of:

(SEQ ID NO. 6)
DVKCDMEVSCPDGYTCCRLQSGAWGCCPFTQAVCCEDHIHCCPAGFTCDTQ
KGTCE

The wild-type human granulin-5 (GRN-5) peptide has the amino acid sequence of:

(SEQ ID NO. 7)
VPCDNVSSCPSSDTCCQLTSGEWGCCPIPEAVCCSDHQHCCPQGYTCVAEG
QCQ

The wild-type human granulin-6 (GRN-6) peptide has the amino acid sequence of:

(SEQ ID NO. 8)
IGCDQHTSCPVGQTCCPSLGGSWACCQLPHAVCCEDRQHCCPAGYTCNVKA
RSCE

The wild-type human granulin-7 (GRN-7) peptide has the amino acid sequence of:

(SEQ ID NO. 9)
DVECGEGHFCHDNQTCCRDNRQGWACCPYRQGVCCADRRHCCPAGFRCAAR
GTKCL

The wild-type human PGRN (CODS identifier: 11483.1) has the nucleic acid sequence of:

(SEQ ID NO. 10)
ATGTGGACCCTGGTGAGCTGGGTGGCCTTAACAGCAGGGCTGGTGGCTGGA
ACGCGGTGCCCAGATGGTCAGTTCTGCCCTGTGGCCTGCTGCCTGGACCCC
GGAGGAGCCAGCTACAGCTGCTGCCGTCCCCTTCTGGACAAATGGCCCACA
ACACTGAGCAGGCATCTGGGTGGCCCCTGCCAGGTTGATGCCCACTGCTCT
GCCGGCCACTCCTGCATCTTTACCGTCTCAGGGACTTCCAGTTGCTGCCCC
TTCCCAGAGGCCGTGGCATGCGGGGATGGCCATCACTGCTGCCCACGGGGC
TTCCACTGCAGTGCAGACGGGCGATCCTGCTTCCAAAGATCAGGTAACAAC
TCCGTGGGTGCCATCCAGTGCCCTGATAGTCAGTTCGAATGCCCGGACTTC
TCCACGTGCTGTGTTATGGTCGATGGCTCCTGGGGGTGCTGCCCCATGCCC
CAGGCTTCCTGCTGTGAAGACAGGGTGCACTGCTGTCCGCACGGTGCCTTC
TGCGACCTGGTTCACACCCGCTGCATCACACCCACGGGCACCCACCCCCTG
GCAAAGAAGCTCCCTGCCCAGAGGACTAACAGGGCAGTGGCCTTGTCCAGC
TCGGTCATGTGTCCGGACGCACGGTCCCGGTGCCCTGATGGTTCTACCTGC
TGTGAGCTGCCCAGTGGGAAGTATGGCTGCTGCCCAATGCCCAACGCCACC
TGCTGCTCCGATCACCTGCACTGCTGCCCCCAAGACACTGTGTGTGACCTG
ATCCAGAGTAAGTGCCTCTCCAAGGAGAACGCTACCACGGACCTCCTCACT
AAGCTGCCTGCGCACACAGTGGGGGATGTGAAATGTGACATGGAGGTGAGC
TGCCCAGATGGCTATACCTGCTGCCGTCTACAGTCGGGGGCCTGGGGCTGC
TGCCCTTTTACCCAGGCTGTGTGCTGTGAGGACCACATACACTGCTGTCCC
GCGGGGTTTACGTGTGACACGCAGAAGGGTACCTGTGAACAGGGGCCCCAC
CAGGTGCCCTGGATGGAGAAGGCCCCAGCTCACCTCAGCCTGCCAGACCCA
CAAGCCTTGAAGAGAGATGTCCCCTGTGATAATGTCAGCAGCTGTCCCTCC
TCCGATACCTGCTGCCAACTCACGTCTGGGGAGTGGGGCTGCTGTCCAATC
CCAGAGGCTGTCTGCTGCTCGGACCACCAGCACTGCTGCCCCCAGGGCTAC
ACGTGTGTAGCTGAGGGGCAGTGTCAGCGAGGAAGCGAGATCGTGGCTGGA
CTGGAGAAGATGCCTGCCCGCCGGGCTTCCTTATCCCACCCCAGAGACATC
GGCTGTGACCAGCACACCAGCTGCCCGGTGGGGCAGACCTGCTGCCCGAGC
CTGGGTGGGAGCTGGGCCTGCTGCCAGTTGCCCCATGCTGTGTGCTGCGAG
GATCGCCAGCACTGCTGCCCGGCTGGCTACACCTGCAACGTGAAGGCTCGA
TCCTGCGAGAAGGAAGTGGTCTCTGCCCAGCCTGCCACCTTCCTGGCCCGT
AGCCCTCACGTGGGTGTGAAGGACGTGGAGTGTGGGGAAGGACACTTCTGC
CATGATAACCAGACCTGCTGCCGAGACAACCGACAGGGCTGGGCCTGCTGT
CCCTACCGCCAGGGCGTCTGTTGTGCTGATCGGCGCCACTGCTGTCCTGCT
GGCTTCCGCTGCGCAGCCAGGGGTACCAAGTGTTTGCGCAGGGAGGCCCCG
CGCTGGGACGCCCCTTTGAGGGACCCAGCCTTGAGACAGCTGCTGTGA

The codon-optimized human PGRN has the nucleic acid sequence of:

(SEQ ID NO. 19)
ATGTGGACTCTGGTCTCATGGGTCGCTCTGACAGCAGGACTGGTCGCAGGA
ACAAGATGCCCCGATGGACAGTTTTGCCCCGTCGCTTGCTGTCTGGACCCA
GGAGGAGCAAGCTACTCCTGCTGTAGGCCACTGCTGGATAAGTGGCCCACC
ACACTGTCCCGCCACCTGGGAGGACCATGCCAGGTGGACGCACACTGTTCC
GCCGGACACTCTTGCATCTTCACAGTGTCTGGCACCAGCTCCTGCTGTCCA
TTTCCTGAGGCAGTGGCATGCGGCGACGGACACCACTGCTGTCCCAGGGGC
TTCCACTGTAGCGCCGATGGCCGGTCCTGCTTTCAGAGAAGCGGCAACAAT
TCCGTGGGCGCCATCCAGTGTCCTGACAGCCAGTTCGAGTGCCCAGATTTT
TCCACCTGCTGCGTGATGGTGGATGGCTCTTGGGGCTGCTGTCCAATGCCC
CAGGCCAGCTGCTGTGAGGACAGGGTGCACTGCTGTCCTCACGGCGCCTTC
TGTGATCTGGTGCACACACGCTGCATCACCCCCACAGGCACCCACCCTCTG
GCCAAGAAGCTGCCAGCACAGAGGACCAACAGGGCAGTGGCCCTGTCTAGC
AGCGTGATGTGCCCCGACGCCCGGTCTAGATGCCCTGATGGCAGCACCTGC
TGTGAGCTGCCAAGCGGCAAGTACGGCTGCTGTCCTATGCCAAACGCCACA
TGCTGTTCCGACCACCTGCACTGCTGTCCTCAGGACACCGTGTGCGATCTG
ATCCAGTCTAAGTGCCTGAGCAAGGAGAATGCCACCACAGACCTGCTGACA
AAGCTGCCTGCCCACACCGTGGGCGACGTGAAGTGTGATATGGAGGTGTCC
TGCCCAGATGGCTATACATGCTGTCGGCTGCAGTCTGGAGCATGGGGATGC
TGTCCCTTCACCCAGGCCGTGTGCTGTGAGGACCACATCCACTGCTGTCCT
GCCGGCTTTACATGCGATACCCAGAAGGGCACATGCGAGCAGGGCCCTCAC
CAGGTGCCATGGATGGAGAAGGCACCAGCACACCTGTCCCTGCCCGACCCT
CAGGCCCTGAAGAGAGACGTGCCTTGTGATAACGTGTCTAGCTGCCCATCC
TCTGATACATGCTGTCAGCTGACCTCTGGCGAGTGGGGCTGCTGTCCAATC
CCCGAGGCCGTGTGCTGTAGCGACCACCAGCACTGCTGTCCTCAGGGCTAT
ACCTGCGTGGCAGAGGGACAGTGCCAGAGGGGCTCCGAGATCGTGGCAGGA
CTGGAGAAGATGCCAGCAAGGAGAGCATCTCTGAGCCACCCCAGAGACATC
GGCTGTGATCAGCACACAAGCTGCCCAGTGGGACAGACCTGCTGTCCATCC
CTGGGAGGCTCTTGGGCATGCTGTCAGCTGCCTCACGCCGTGTGCTGTGAG
GATCGGCAGCACTGCTGTCCAGCCGGCTACACATGCAATGTGAAGGCCAGA
TCCTGCGAGAAGGAGGTGGTGTCTGCCCAGCCAGCCACCTTCCTGGCAAGG
AGCCCTCACGTGGGAGTGAAGGACGTGGAGTGTGGCGAGGGCCACTTTTGC
CACGACAACCAGACATGCTGTCGGGATAATAGACAGGGCTGGGCCTGCTGT
CCATATAGGCAGGGCGTGTGCTGTGCAGATAGGCGCCACTGCTGTCCAGCA
GGCTTTAGGTGCGCAGCAAGGGGAACCAAGTGCCTGAGAAGAGAAGCCCCC
CGGTGGGACGCCCCCCTGAGAGACCCTGCCCTGAGACAGCTGCTGTGATGA

According to the methods described herein, a subject can be administered a cell (e.g., pluripotent cells, ESCs, iPSCs, multipotent cells, CD34+ cells, HSCs, MPCs, BLPCs, monocytes, macrophages, microglial progenitor cells, or microglia) that contains a polynucleotide encoding the amino acid sequence of SEQ ID NO. 1, or a polynucleotide encoding a polypeptide having at least 85% sequence identity (e.g., at least 85%, 90%, 95%, 96%, 97%, 98%, 99%, or more, sequence identity) to the amino acid sequence of SEQ ID NO. 1, or a polynucleotide encoding a polypeptide that contains one or more conservative amino acid substitutions relative to SEQ ID NO. 1 (e.g., 1, 2, 3, 4, 5, 6, 7, 8, 9, or 10 or more conservative amino acid substitutions), or a polynucleotide encoding a polypeptide that contains 1 or more GRN domains having an amino acid sequence of any one of SEQ ID NOs. 2-9 or a variant of having at least 85% sequence identity (e.g., at least 85%, 90%, 95%, 96%, 97%, 98%, 99%, or more, sequence identity) to any one of SEQ ID NOs. 2-9, provided that the PGRN or the GRN variant encoded retains the therapeutic function of the wild-type PGRN or GRN. The neurotrophic activity of the wild-type PGRN is important for the neurotrophic support and maintenance of lysosomal function. Loss of PGRN leads to neurodegeneration and lysosomal storage disease in a dose-dependent manner.

### Host Cells

Cells that may be used in conjunction with the compositions and methods described herein include cells (e.g., pluripotent cells, ESCs, iPSCs, CD34+ cells, HSCs, MPCs, BLPCs, monocytes, or microglial progenitor cells) or differentiated cells (e.g., macrophages or microglia). For example, one type of cell that can be used in conjunction with the compositions and methods described herein is a pluripotent cell. A pluripotent cell is a cell that possesses the ability to develop into more than one differentiated cell type. Examples of pluripotent cells are ESCs and iPSCs. ESCs and iPSCs have the ability to differentiate into cells of the ectoderm, which gives rise to the skin and nervous system, endoderm, which forms the gastrointestinal and respiratory tracts, endocrine glands, liver, and pancreas, and mesoderm, which forms bone, cartilage, muscles, connective tissue, and most of the circulatory system. Another type of cell that can be used in conjunction with the compositions and methods described herein is a multipotent cell. A multipotent cell is a cell that possesses the ability to differentiate into multiple, but not all cell types. A non-limiting example of a multipotent cell is a CD34+ cell (e.g., HSCs or MPC).

Cells that may be used in conjunction with the compositions and methods described herein include HSCs and MPCs. HSCs are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Human HSCs are CD34+. In addition, HSCs also refer to long term repopulating HSC (LT-HSC) and short-term repopulating HSC (ST-HSC). Any of these HSCs can be used in conjunction with the compositions and methods described herein.

HSCs can differentiate into myeloid progenitor cells, which are also CD34+. Myeloid progenitors can further differentiate into granulocytes (e.g., promyelocytes, neutrophils, eosinophils, and basophils), erythrocytes (e.g., reticulocytes, erythrocytes), thrombocytes (e.g., megakaryoblasts, platelet producing megakaryocytes, and platelets), monocytes (e.g., monocytes and macrophages), dendritic cells, and microglia. Common myeloid progenitors can be characterized by cell surface molecules and are known to be lin−, SCA1−, c-kit+, CD34+, and CD16/32^{mid}.

HSCs and myeloid progenitors can be obtained from blood products. A blood product is a product obtained from the body or an organ of the body containing cells of hematopoietic origin. Such sources include unfractionated bone marrow, umbilical cord, placenta, peripheral blood, or mobilized peripheral blood. All of the aforementioned crude or unfractionated blood products can be enriched for cells having HSC or myeloid progenitor cell characteristics in a number of ways. For example, the more mature, differentiated cells can be selected against based on cell surface molecules they express. The blood product may be fractionated by positively selecting for CD34+ cells, which include a subpopulation of hematopoietic stem cells capable of self-renewal, multi-potency, and that can be re-introduced into a transplant recipient whereupon they home to the hematopoietic stem cell niche and reestablish productive and sustained hematopoiesis. Such selection is accomplished using, for example, commercially available magnetic anti-CD34 beads (Dynal, Lake Success, NY). Myeloid progenitor cells can also be isolated based on the markers they express. Unfractionated blood products can be obtained directly from a donor or retrieved from cryopreservative storage. HSCs and myeloid progenitor cells can also be obtained from by differentiation of ES cells, iPS cells or other reprogrammed mature cells types.

Cells that may be used in conjunction with the compositions and methods described herein include allogeneic cells and autologous cells. All of the aforementioned cell types are capable of differentiating into microglia. Cells described herein may also differentiate into microglial progenitors or microglial stem cells. Differentiation may occur ex vivo or in vivo. Methods for ex vivo differentiation of human ESCs and iPSCs are known by those of skill in the art and are described in Muffat et al., Nature Medicine 22:1358-1367 (2016) and Pandya et al., Nature Neuroscience (2017) epub ahead of print, the disclosures of which are incorporated herein by reference as they pertain to methods of differentiating cells into microglia.

### Microglia

Cells that may be used in conjunction with the compositions and methods described herein include those that are capable of differentiating into microglial cells or cells that are differentiated microglial cells. Microglia are myeloid-derived cells that serve as the immune cells, or resident macrophages, of the central nervous system. Microglia are highly similar to macrophages, both genetically and functionally, and share the ability to shift dynamically between pro-inflammatory and anti-inflammatory states. The pro-inflammatory state is known as classical activation, or M1, and the anti-inflammatory state is called alternative activation, or M2. Microglia can be made to shift between the two states by extracellular signals, e.g., signals from neighboring neurons or astrocytes, cell debris, toxins, infection, ischemia, and traumatic injury, among others. M1 microglia are often observed in the diseased brain, particularly in diseases involving neuroinflammation, such as FTLD or NCL. Classically activated M1 phenotypes have also been observed in mouse models of FTLD and NCL, such as the progranulin null mouse, the CLN3(Δex7/8) mouse, and the CLN6^{ncif} mouse. It is unclear whether M1 microglia are a cause or consequence of neuroinflammation, but once microglia are classically activated, they can secrete pro-inflammatory cytokines, e.g., TNF-α, IL-1β, and IL-6, chemokines, and nitric oxide, which can lead to sustained inflammation, neuronal damage, and further activation of M1 microglia. This positive feedback loop can be harmful to brain tissue; therefore, methods of reducing M1 activation and/or increasing M2 activation may help patients with diseases featuring neuroinflammation, e.g., an NCD.

### Expression of Progranulin or Granulin in Mammalian Cells

PGRN activity is reduced in patients with FTLD and NCL, and FTLD brains contain classically activated M1 microglia. The compositions and methods described herein target these dysfunctions by administering cells (e.g., pluripotent cells, ESCs, iPSCs, multipotent cells, CD34+ cells, HSCs, MPCs, BLPCs, monocytes, macrophages, microglial progenitor cells, or microglia) containing a transgene (e.g., a transgene capable of expression in macrophages or microglial cells) encoding a PGRN or a GRN. In order to utilize these agents for therapeutic application in the treatment of an NCD (e.g., FTLD or NCL), these agents can be directed to the interior of the cell, and in particular examples, to particular organelles. A wide array of methods has been established for the delivery of such proteins to mammalian cells and for the stable expression of genes encoding such proteins in mammalian cells.

### Polynucleotides Encoding Progranulin or Granulin

One platform that can be used to achieve therapeutically effective intracellular concentrations of PGRN or GRN in mammalian cells (e.g., pluripotent cells, ESCs, iPSCs, multipotent cells, CD34+ cells, HSCs, MPCs, BLPCs, monocytes, macrophages, microglial progenitor cells, or microglia) is via the stable expression of genes encoding these agents (e.g., by integration into the nuclear or mitochondrial genome of a mammalian cell). These genes are polynucleotides that encode the primary amino acid sequence of the corresponding protein. In order to introduce such exogenous genes into a mammalian cell, these genes can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

The PGRN or the GRN can also be introduced into a mammalian cell by targeting a vector containing a gene encoding such an agent to cell membrane phospholipids. For example, vectors can be targeted to the phospholipids on the extracellular surface of the cell membrane by linking the vector molecule to a VSV-G protein, a viral protein with affinity for all cell membrane phospholipids. Such, a construct can be produced using methods well known to those of skill in the field.

Recognition and binding of the polynucleotide encoding the PGRN or the GRN by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith et al., Mol. Sys. Biol., 3:73 (2007), online publication, the disclosure of which is incorporated herein by reference.

Polynucleotides suitable for use with the compositions and methods described herein also include those that encode the PGRN or the GRN downstream of a mammalian promoter. Promoters that are useful for the expression of the PGRN or the GRN in mammalian cells include, e.g., elongation factor 1-alpha (EF1α) promoter, phosphoglycerate kinase 1 (PGK) promoter, CD68 molecule (CD68) promoter (see Dahl et al., Molecular Therapy 23:835 (2015), incorporated herein by reference as it pertains to the use of PGK and CD68 promoters to express PGRN), CD11 b promoter, PGRN promoter, C-X3-C motif chemokine receptor 1 (CX3CR1) promoter, allograft inflammatory factor 1 (AIF1) promoter, purinergic receptor P2Y12 (P2Y12) promoter, transmembrane protein 119 (TMEM119) promoter, and colony stimulating factor 1 receptor (CSF1R) promoter. Alternatively, promoters derived from viral genomes can also be used for the stable expression of these agents in mammalian cells. Examples of functional viral promoters that can be used to promote mammalian expression of these agents are adenovirus late promoter, vaccinia virus 7.5K promoter, simian virus 40 (SV40) promoter, cytomegalovirus promoter, tk promoter of herpes simplex virus (HSV), mouse mammary tumor virus (MMTV) promoter, long terminal repeat (LTR) promoter of human immunodeficiency virus (HIV), promoter of moloney virus, Epstein barr virus (EBV), Rous sarcoma virus (RSV), and the cytomegalovirus (CMV) promoter. Alternatively, synthetic promoters optimized for use in mammalian cells can be employed for stable expression of PGRN or GRN transgenes.

Once a polynucleotide encoding the PGRN or the GRN has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, Calif.) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode the PGRN or the GRN and additionally include a mammalian enhancer sequence. Many enhancer sequences are now known from mammalian genes, and examples are enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982). An enhancer may be spliced into a vector containing a polynucleotide encoding a water-forming NADH oxidase, for example, at a position 5′ or 3′ to this gene. In a preferred orientation, the enhancer is positioned at the 5′ side of the promoter, which in turn is located 5′ relative to the polynucleotide encoding the PGRN or the GRN.

### Cell-Specific Gene Expression

Interfering RNA (RNAi) are widely used to knock down the expression of endogenous genes by delivering small interfering RNA (siRNA) into cells triggering the degradation of complementary mRNA. An additional application is to utilize the diversity of endogenous micro RNAs (miRNA) to negatively regulate the expression of exogenously introduced transgenes tagged with artificial miRNA target sequences. These miRNA target tagged transgenes can be negatively regulated according to the activity of a given miRNA which can be tissue, lineage, activation, or differentiation stage specific. These artificial miRNA target sequences (miRTs) can be recognized as targets by a specific miRNA thus inducing post-transcriptional gene silencing. While robust transgene expression in targeted cells can have beneficial therapeutic results, off target expression, such as the ectopic or non-regulated transgene expression in HSPCs or other progenitor cells, can have cytotoxic effects, which can result in counter-selection of transgene-containing cells leading to altered cellular behavior and reduced therapeutic efficacy. The incorporation of miRTs for miRNAs widely expressed in HSPCs and progenitors, but absent in cells of the myeloid lineage can allow for repressed transgene expression in HSPCs and other progenitor cells allowing for silent, long-term reservoir transgene-containing hematopoietic progeny, while allowing for robust transgene expression in differentiated, mature target cells. miR-126 is highly expressed in HSPCs, other progenitor cells, and cells of the erythroid lineage, but absent from those of the myeloid lineage (e.g., macrophages and microglia) (Gentner et al., Science Translational Medicine 2:58ra34 (2010)). A miR-126 targeting sequence, for example, incorporated within a transgene would allow for targeted expression of the transgene in cells of the myeloid lineage and repressed expression in HSPCs and other progenitor cells, thus minimizing off-target cytotoxic effects. In some embodiments, the transgene encoding the PGRN or the GRN agent may include a miR-126 targeting sequence.

### Secretory Signal Peptides

Polynucleotides encoding the PGRN or the GRN may include one or more polynucleotides encoding a secretory signal peptide. Secretory signal peptides may have amino acid sequences of 5-30 residues in length, and may be located upstream of (i.e., 5′ to) a polynucleotide encoding the PGRN or the GRN. These secretory signal peptides allow for the recognition of the nascent polypeptides during synthesis by signal recognition particles resulting in translocation to the ER, packaging into transport vesicles, and finally, secretion. Exemplary secretory signal peptides for protein secretion are those from PGRN, IGF-II, alpha-1 antitrypsin, IL-2, IL-6, CD5, immunoglobulins, trypsinogen, serum albumin, prolactin, elastin, tissue plasminogen activator signal peptide (tPA-SP), and insulin. In some embodiments, cells (e.g., pluripotent cells, ESCs, iPSCs, multipotent cells, CD34+ cells, HSCs, MPCs, BLPCs, monocytes, macrophages, microglial progenitor cells, or microglia) containing a transgene encoding a secreted form of the PGRN or the GRN may be utilized as a therapeutic strategy to correct a protein deficiency (e.g., PGRN or GRN) by infusing the missing protein into the bloodstream. As the blood perfuses patient tissues, the PGRN or the GRN is taken up by cells and transported to its site of action.

### ApoE Tag for Blood-Brain Barrier Penetrance of Secreted Progranulin or Granulin

In some embodiments, the PGRN or the GRN (e.g., the PGRN or the GRN fusion protein) is modified to penetrate the blood-brain barrier (BBB). Modifications for mediating BBB penetrance are well known in the art. Exemplary modifications are the use of tags containing the Rb domain (amino acid residues 148-173 of SEQ ID NO. 11) of ApoE. The complete ApoE peptide sequence is shown below.

(SEQ ID NO. 11)
MKVLWAALLVTFLAGCQAKVEQAVETEPEPELRQQTEWQSGQRWELALGRF
WDYLRWVQTLSEQVQEELLSSQVTQELRALMDETMKELKAYKSELEEQLTP
VAEETRARLSKELQAAQARLGADMEDVCGRLVQYRGEVQAMLGQSTEELRV
RLASHLRKLRKRLLRDADDLQKRLAVYQAGAREGAERGLSAIRERLGPLVE
QGRVRAATVGSLAGQPLQERAQAWGERLRARMEEMGSRTRDRLDEVKEQVA
EVRAKLEEQAQQIRLQAEAFQARLKSWFEPLVEDMQRQWAGLVEKVQAAVG
TSAAPVPSDNH

ApoE is an important protein involved in lipid transport, and its cellular internalization is mediated by several members of the low-density lipoprotein (LDL) receptor gene family, including the LDL receptor, very low-density lipoprotein receptor (VLDLR), and LDL receptor-related proteins (LRPs, including LRP1, LRP2, and LRP8). The LDL receptor is found to be highly expressed in brain capillary endothelial cells (BCECs), with down-regulated expression observed in peripheral vessels. Restricted expressions of LRPs and VLDLR have also been shown prominently in the liver and brain when they have been detected in BCECs, neurons, and glial cells. Several members of the low-density lipoprotein receptor family (LDLRf) proteins, including LRP1 and VLDLR, but not LDLR, are highly expressed in BBB-forming BCECs. These proteins can bind ApoE to facilitate their transcytosis into the abluminal side of the BBB.

In addition, receptor-associated protein (RAP), an antagonist as well as a ligand for both LRP1 and VLDLR, has been shown to have higher permeability across the BBB than transferrin in vivo and in vitro (Pan et al., J. Cell Sci. 117:5071-8 (2004)), indicating that these lipoprotein receptors (LDLRf) can represent efficient BBB delivery targets despite their lower expression than the transferrin receptor. As described herein, a potent receptor-binding peptide (Rb) derived from ApoE, has the ability to translocate protein across the BBB into the brain when engineered as fusion proteins. This method can therefore function to selectively open the BBB for therapeutic agents when engineered as a fusion protein. This peptide can be readily attached to diagnostic or therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Regardless of application strategies, e.g., enzyme replacement therapy or cell-based, gene-based therapy, both the quantity and distribution of therapeutics within the brain parenchyma will have a significant impact on the clinical outcome of disease treatment. The development of and a detailed description of the use of the Rb domain of ApoE in targeted delivery of proteins across the BBB can be found in U.S. Publication No. 20140219974, which is hereby incorporated by reference in its entirety.

In some embodiments, the PGRN or the GRN fusion protein has a peptide sequence containing the LDLRf Rb domain of SEQ ID NO. 11, or a fragment, variant, or oligomer thereof. An exemplary receptor-binding domain can be found in the N-terminus of ApoE, for example, between amino acid residues 1 to 191 of SEQ ID NO. 11, between amino acid residues 25 to 185 of SEQ ID NO. 11, between amino acid residues 50 to 180 of SEQ ID NO. 11, between amino acid residues 75 to 175 of SEQ ID NO. 11, between amino acid residues 100 to 170 of SEQ ID NO. 11, or between amino acid residues 125 to 165 of SEQ ID NO. 11. An exemplary receptor binding domain has the amino acid sequence of residues 159 to 167 of SEQ ID NO. 11.

In some embodiments, the peptide sequence containing the receptor-binding domain of ApoE can include at least one amino acid mutation, deletion, addition, or substitution. In some embodiments, the amino acid substitutions can be a combination of two or more mutations, deletions, additions, or substitutions. In some embodiments, the at least one substation is a conservative substitution. In some embodiments, the at least one amino acid addition includes addition of a selected sequence already found in the Rb domain of ApoE. A person of ordinary skill in the art will recognize suitable modifications that can be made to the sequence while retaining some degree of the biochemical activity for transport across the BBB.

### Glycosylation Independent Lysosomal Targeting

Glycosylation Independent Lysosomal Targeting (GILT) technology can be utilized to target therapeutic enzymes (e.g., PGRN or GRN) to lysosomes. Specifically, the GILT technology uses a peptide tag instead of M6P to engage the CI-MPR for lysosomal targeting. Typically, a GILT tag is a protein, peptide, or other moiety that binds the CI-MPR in a mannose-6-phosphateindependent manner. Advantageously, this technology mimics the normal biological mechanism for uptake of lysosomal enzymes yet does so in a manner independent of mannose-6-phosphate. In some embodiments, the PGRN or GRN is secreted as a PGRN or GRN fusion protein containing PGRN or GRN and a GILT tag. In some embodiments, the GILT tag is fused to the N-terminus of the PGRN or GRN protein. In some embodiments, the GILT tag is fused to the C-terminus of the PGRN or GRN protein. In some embodiments, a GILT tag is derived from human insulin-like growth factor II (IGFII). Human IGF-II is a high affinity ligand for the CI-MPR; also referred to as IGF-II receptor. Binding of GILT-tagged therapeutic enzymes to the M6P/IGF-II receptor targets the protein to the lysosome via the endocytic pathway. A detailed description of GILT technology and the GILT tag can be found in U.S. Publication Nos. 20030082176, 20040006008, 20040005309, 20050281805, and 2009043207 the teachings of all of which are hereby incorporated by references in their entireties.

### Furin-Resistant GILT Tag

The IGF-II derived GILT tag may be subjected to proteolytic cleavage by furin during production in mammalian cells. Furin protease typically recognizes and cleaves a cleavage site having a consensus sequence Arg-X-X-Arg, where X is any amino acid. The cleavage site is positioned after the carboxy-terminal arginine (Arg) residue in the sequence. In some embodiments, a furin cleavage site has a consensus sequence Lys/Arg-X-X-X-Lys/Arg-Arg, where X is any amino acid. The cleavage site is positioned after the carboxy terminal arginine (Arg) residue in the sequence. The mature human IGF-II peptide sequence is shown below.

(SEQ ID NO. 12)
AYRPSETLCGGELVDTLQFVCGDRGFYFSRPASRVSRRSRGIVEECCFRSC
DLALLETYCATPAKSE

The mature human IGF-II contains two potential overlapping furin cleavage sites between residues 34-40 (bolded). Modified GILT tags that are resistant to cleavage by furin still retain ability to bind to the CI-MPR in a mannose-6-phosphate-independent manner. Specifically, furin-resistant GILT tags can be designed by mutating the amino acid sequence at one or more furin cleavage sites such that the mutation abolishes at least one furin cleavage site. Thus, in some embodiments, a furin-resistant GILT tag is a furin-resistant IGF-II mutein containing a mutation that abolishes at least one furin protease cleavage site or changes a sequence adjacent to the furin protease cleavage site such that the furin cleavage is prevented, inhibited, reduced or slowed down as compared to a wild-type IGF-II peptide (e.g., wild-type human mature IGF-II). A suitable mutation does not impact the ability of the furin-resistant GILT tag to bind to the human cation-independent mannose-6-phosphate receptor. In some embodiments, a furin-resistant IGF-II mutein suitable for use in conjunction with the compositions and methods described herein binds to the human cation-independent mannose-6-phosphate receptor in a mannose-6-phosphate-independent manner with a dissociation constant of 10-7 M or less (e.g., 10-8, 10-9, 10-10, 10-11, or less) at pH 7.4. In some embodiments, a furin-resistant IGF-II mutein contains a mutation within a region corresponding to amino acids 30-40 (e.g., 31-40, 32-40, 33-40, 34-40, 30-39, 31-39, 32-39, 34-37, 32-39, 33-39, 34-39, 35-39, 36-39, 37-40, 34-40) of SEQ ID NO. 12. In some embodiments, a suitable mutation abolishes at least one furin protease cleavage site. A mutation can be amino acid substitutions, deletions, or insertions. For example, any one amino acid within the region corresponding to residues 30-40 (e.g., 31-40, 32-40, 33-40, 34-40, 30-39, 31-39, 32-39, 34-37, 32-39, 33-39, 34-39, 35-39, 36-39, 37-40, 34-40) of SEQ ID NO. 12 can be substituted with any other amino acid or deleted. For example, substitutions at position 34 may affect furin recognition of the first cleavage site. Insertion of one or more additional amino acids within each recognition site may abolish one or both furin cleavage sites. Deletion of one or more of the residues in the degenerate positions may also abolish both furin cleavage sites.

In some embodiments, a furin-resistant IGF-II mutein contains amino acid substitutions at positions corresponding to Arg37 or Arg40 of SEQ ID NO. 12. In some embodiments, a furin-resistant IGF-II mutein contains a Lys or Ala substitution at positions Arg37 or Arg40. Other substitutions are possible, including combinations of Lys and/or Ala mutations at both positions 37 and 40, or substitutions of amino acids other than Lys or Ala. In some embodiments, the furin-resistant IGF-II mutein suitable for use in conjunction with the compositions and methods described herein may contain additional mutations. For example, up to 30% or more of the residues of SEQ ID NO. 12 may be changed (e.g., up to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or more residues may be changed). Thus, a furin-resistant IGF-II mutein suitable for use in conjunction with the compositions and methods described herein may have an amino acid sequence at least 70%, including at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO. 12. In some embodiments, a furin-resistant IGF-II mutein suitable for use in conjunction with the compositions and methods described herein is targeted specifically to the CI-MPR. Particularly useful are mutations in the IGF-II polypeptide that result in a protein that binds the CI-MPR with high affinity (e.g., with a dissociation constant of 10-7 M or less at pH 7.4) while binding other receptors known to be bound by IGF-II with reduced affinity relative to native IGF-II. For example, a furin-resistant IGF-II mutein suitable for use in conjunction with the compositions and methods described herein can be modified to have diminished binding affinity for the IGF-I receptor relative to the affinity of naturally-occurring human IGF-II for the IGF-I receptor. Additional mutational strategies have been utilized and are discussed at length in the U.S. Publication No. 2009043207, which is hereby incorporated by reference. For example, substitution of IGF-II residues Tyr 27 with Leu, Leu 43 with Val, or Ser 26 with Phe diminishes the affinity of IGF-II for the IGF-I receptor by 94-, 56-, and 4-fold respectively (Torres et al., J. Mol. Biol. 248(2):385-401 (1995)). Deletion of residues 1-7 of human IGF-II resulted in a 30-fold decrease in affinity for the human IGF-I receptor and a concomitant 12-fold increase in affinity for the rat IGF-II receptor (Hashimoto et al., J. Biol. Chem. 270(30)18013-8 (1995)). The NMR structure of IGF-II shows that Thr 7 is located near residues 48 Phe and 50 Ser, as well as near the 9 Cys-4 7 Cys disulfide bridge. It is thought that interaction of Thr 7 with these residues can stabilize the flexible N-terminal hexapeptide required for IGF-I receptor binding (Terasawa et al., EMBO J. 13(23)5590-7 (1994)). At the same time, this interaction can modulate binding to the IGF-II receptor. Truncation of the C-terminus of IGF-II (residues 62-67) also appears to lower the affinity of IGF-II for the IGF-I receptor by 5-old (Roth et al., Biochem. Biophys. Res. Commun. 181(2):907-14 (1991)). The binding surfaces for the IGF-I and cation-independent M6P receptors are on separate faces of IGF-II. Based on structural and mutational data, functional cation-independent M6P binding domains can be constructed that are substantially smaller than human IGF-II. For example, the amino terminal amino acids (e.g., 1-7 or 2-7) and/or the carboxy terminal residues 62-67 can be deleted or replaced. Additionally, amino acids 29-40 can likely be eliminated or replaced without altering the folding of the remainder of the polypeptide or binding to the cation-independent M6P receptor. Thus, a targeting moiety including amino acids 8-28 and 41-61 can be constructed. These stretches of amino acids could perhaps be joined directly or separated by a linker. Alternatively, amino acids 8-28 and 41-61 can be provided on separate polypeptide chains. Comparable domains of insulin, which are homologous to IGF-II and have a tertiary structure closely related to the structure of IGF-II, have sufficient structural information to permit proper refolding into the appropriate tertiary structure, even when present in separate polypeptide chains (Wang et al., Trends Biochem. Sci. 16(8):279-281 (1991)). Thus, for example, amino acids 8-28, or a conservative substitution variant thereof, could be fused to a lysosomal enzyme; the resulting fusion protein could be admixed with amino acids 41-61, or a conservative substitution variant thereof, and administered to a patient. IGF-II can also be modified to minimize binding to serum IGF-binding proteins (Baxter, Am. J. Physiol Endocrinol Metab. 278(6):967-76(2000)) to avoid sequestration of IGF-II/GILT constructs. A number of studies have localized residues in IGF-II necessary for binding to IGF-binding proteins. Constructs with mutations at these residues can be screened for retention of high affinity binding to the M6P/IGF-II receptor and for reduced affinity for IGF binding proteins. For example, replacing Phe 26 of IGF-II with Ser is reported to reduce affinity of IGF-II for IGFBP-1 and -6, with no effect on binding to the M6P/IGF-II receptor (Bach et al., J. Biol. Chem. 268(13):9246-54 (1993)). Other substitutions, such as Lys for Glu 9, can also be advantageous. The analogous mutations, separately or in combination, in a region of IGF-I that is highly conserved with IGF-II result in large decreases in IGF-BP binding (Magee et al., Biochemistry 38(48):15863-70 (1999)).

An alternate approach is to identify minimal regions of IGF-II that can bind with high affinity to the M6P/IGF-II receptor. The residues that have been implicated in IGF-II binding to the M6P/IGF-II receptor mostly cluster on one face of IGF-II (Terasawa et al., EMBO J. 13(23):5590-7 (1994)). Although IGF-II tertiary structure is normally maintained by three intramolecular disulfide bonds, a peptide incorporating the amino acid sequence on the M6P/IGF-II receptor binding surface of IGF-II can be designed to fold properly and have binding activity. Such a minimal binding peptide is a highly preferred lysosomal targeting domain. For example, a preferred lysosomal targeting domain is amino acids 8-67 of human IGF-II. Designed peptides, based on the region around amino acids 48-55, which bind to the M6P/IGF-II receptor, are also desirable lysosomal targeting domains. Alternatively, a random library of peptides can be screened for the ability to bind the M6P/IGF-II receptor either via a yeast two hybrid assay, or via a phage display type assay.

Many furin-resistant IGF-II muteins described herein have reduced or diminished binding affinity for the insulin receptor. Thus, in some embodiments, a peptide tag suitable for use in conjunction with the compositions and methods described herein has reduced or diminished binding affinity for the insulin receptor relative to the affinity of naturally occurring human IGF-II for the insulin receptor. In some embodiments, peptide tags with reduced or diminished binding affinity for the insulin receptor suitable for use in conjunction with the compositions and methods described herein include peptide tags having a binding affinity for the insulin receptor that is more than 1.5-fold, 2-fold, 3-fold, 4-fold, 5-fold, 6-fold, 7-fold, 8-fold, 9-fold, 10-fold, 12-fold, 14-fold, 16-fold, 18-fold, 20-fold, 50-fold, 100-fold less than that of the wild-type mature human IGF-II. The binding affinity for the insulin receptor can be measured using various in vitro and in vivo assays known in the art.

In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 13, as shown below.

(SEQ ID NO. 13)
GGGGAGGGGAGGGGAGGGGAGGGPSLCGGELVDTLQFVCGDRGFYFSRPAS
RVSARSRGIVEECCFRSCDLALLETYCATPAKSE

In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 14, as shown below.

(SEQ ID NO. 14)
GAPGGGSPAPAPTPAPAPTPAPAGGGPSGAPLCGGELVDTLQFVCGDRGFY
FSRPASRVSARSRGIVEECCFRSCDLALLETYCATPAKSE

In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 15, as shown below.

(SEQ ID NO. 15)
GAPGGSPAGSPTSTEEGTSESATPESGPGTSTEPSEGSAPGSPAGSPTSTG
PSGAPLCGGELVDTLQFVCGDRGFYFSRPASRVSARSRGIVEECCFRSCDL
ALLETYCATPAKSE

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO. 16, as shown below.

(SEQ ID NO. 16)
GGCGGAGGCGGAGCTGGTGGCGGCGGAGCAGGCGGTGGTGGTGCAGGCGGC
GGAGGTGCTGGCGGAGGACCATCTCTTTGTGGCGGAGAACTGGTGGACACC
CTGCAGTTCGTGTGTGGCGACAGAGGCTTCTACTTTAGCAGACCCGCCAGC
AGAGTGTCCGCCAGATCTAGAGGAATCGTGGAAGAGTGCTGCTTCAGAAGC
TGCGACCTGGCACTGCTGGAAACCTACTGTGCCACACCAGCCAAGAGCGAG
TGATG

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO. 17, as shown below.

(SEQ ID NO. 17)
GGAGCACCAGGCGGAGGATCTCCAGCTCCTGCTCCTACACCAGCTCCAGCA
CCGACGCCTGCTCCAGCTGGCGGAGGACCTTCTGGTGCACCTCTTTGTGGC
GGAGAGCTGGTGGATACCCTGCAGTTCGTGTGTGGCGACCGGGGCTTCTAC
TTTAGCAGACCTGCCAGCAGAGTGTCCGCCAGATCTAGAGGCATCGTGGAA
GAGTGCTGCTTCAGAAGCTGCGACCTGGCACTGCTGGAAACCTACTGTGCC
ACACCAGCCAAGAGCGAGTGATGA

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO. 18, as shown below.

(SEQ ID NO. 18)
GGAGCACCAGGCGGATCTCCAGCAGGATCTCCAACCTCTACCGAGGAAGGC
ACAAGCGAGTCTGCCACACCTGAGTCTGGACCTGGCACAAGCACAGAGCCT
AGCGAAGGATCTGCCCCAGGTTCTCCTGCCGGCTCTCCTACAAGTACAGGA
CCTTCTGGCGCTCCACTGTGTGGCGGAGAACTGGTGGATACCCTGCAGTTC
GTGTGCGGCGACAGAGGCTTCTACTTTAGCAGACCCGCCAGCAGAGTGTCC
GCCAGATCTAGAGGAATCGTGGAAGAGTGCTGCTTCAGAAGCTGCGATCTG
GCACTGCTGGAAACCTACTGTGCCACACCAGCCAAGAGCGAGTGATGA

### Vectors for the Expression of Progranulin or Granulin

In addition to achieving high rates of transcription and translation, stable expression of an exogenous gene in a mammalian cell (e.g., pluripotent cell, ESC, iPSC, multipotent cell, CD34+ cell, HSC, MPC, BLPC, monocyte, macrophage, microglial progenitor cell, or microglial cell) can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide sequence that encodes a PGRN or a GRN, as well as, e.g., additional sequence elements used for the expression of these agents and/or the integration of these polynucleotide sequences into the genome of a mammalian cell. Certain vectors that can be used for the expression of the PGRN or the GRN include plasmids that contain regulatory sequences, such as promoter and enhancer regions, which direct gene transcription. Other useful vectors for expression of the PGRN or the GRN contain polynucleotide sequences that enhance the rate of translation of these genes or improve the stability or nuclear export of the mRNA that results from gene transcription. These sequence elements include, e.g., 5′- and 3′-untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin.

### Viral Vectors for Expression of Progranulin or Granulin

Viral genomes provide a rich source of vectors that can be used for the efficient delivery of exogenous genes into a mammalian cell (e.g., pluripotent cell, ESC, iPSC, multipotent cell, CD34+ cell, HSC, MPC, BLPC, monocyte, macrophage, microglial progenitor cell, or microglial cell). Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, The Journal of Gene Medicine 6:S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). It will be readily apparent to one skilled in the art that optionally one or more of these regions is substituted with another region performing a similar function.

PGRN or GRN is required to be expressed at sufficiently high levels. Transgene expression is mediated by a promoter sequence. Optionally, the LV includes a CMV promoter. The promoter may also be EF1α or PGK promoter. In another embodiment, the promoter is a microglia-specific promoter, e.g., CD68 promoter, CX3CR1 promoter, CD11 b promoter, AIF1 promoter, P2Y12 promoter, TMEM119 promoter, or CSF1R promoter. Optionally, the LV includes a synthetic promoter optimized for use in mammalian cells. A person skilled in the art will be familiar with a number of promoters that will be suitable in the vector constructs described herein.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al. Gene Ther. 8:811 (2001), Osborn et al. Molecular Therapy 12:569 (2005), Szymczak and Vignali Expert Opin Biol Ther. 5:627 (2005), and Szymczak et al. Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

### Viral Regulatory Elements

The viral regulatory elements are components of delivery vehicles used to introduce nucleic acid molecules into a host cell (e.g., pluripotent cell, ESC, iPSC, multipotent cell, CD34+ cell, HSC, MPC, BLPC, monocyte, macrophage, microglial progenitor cell, or microglial cell). The viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions. One skilled in the art would also appreciate that as other viral regulatory elements are identified, these may be used with the nucleic acid molecules described herein.

### Adeno-Associated Viral Vectors for Nucleic Acid Delivery

Nucleic acids of the compositions and methods described herein may be incorporated into rAAV vectors and/or virions in order to facilitate their introduction into a cell (e.g., pluripotent cell, ESC, iPSC, multipotent cell, CD34+ cell, HSC, MPC, BLPC, monocyte, macrophage, microglial progenitor cell, or microglial cell). AAV vectors can be used in the central nervous system, and appropriate promoters and serotypes are discussed in Pignataro et al., J Neural Transm (2017), epub ahead of print, the disclosure of which is incorporated herein by reference as it pertains to promoters and AAV serotypes useful in CNS gene therapy. rAAV vectors useful in the compositions and methods described herein are recombinant nucleic acid constructs that include (1) a heterologous sequence to be expressed (e.g., a polynucleotide encoding the PGRN or the GRN) and (2) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10 and rh74. For targeting cells located in or delivered to the central nervous system, AAV2, AAV9, and AAV10 may be particularly useful. Construction and use of AAV vectors and AAV proteins of different serotypes are described, for example, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype pseudotyped with a capsid gene derived from a serotype other than the given serotype (e.g., AAV1, AAV2, AAV3, AAV4, AAV5, AAV6, AAV7, AAV8, AAV9, and AAV10, among others). Techniques involving the construction and use of pseudotyped rAAV virions are known in the art and are described, for example, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet. 25:436 (2000) and Kolman and Stemmer, Nat. Biotechnol. 19:423 (2001).

### Methods for the Delivery of Exogenous Nucleic Acids to Target Cells

Techniques that can be used to introduce a polynucleotide, such as codon-optimized DNA or RNA (e.g., mRNA, tRNA, siRNA, miRNA, shRNA, chemically modified RNA) into a mammalian cell (e.g., pluripotent cell, ESC, iPSC, multipotent cell, CD34+ cell, HSC, MPC, BLPC, monocyte, macrophage, microglial progenitor cell, or microglial cell) are well known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:25 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Modulation of Gene Expression Using Gene Editing Techniques

### Disruption of Endogenous Progranulin or Granulin

In some embodiments, endogenous PGRN or GRN is disrupted (e.g., in a subject undergoing treatment, such as in a population of neurons in a subject undergoing treatment, or in the cells (e.g., pluripotent cells, ESCs, iPSCs, multipotent cells, CD34+ cells, HSCs, MPCs, BLPCs, monocytes, macrophages, microglial progenitor cells, or microglia) to be administered to the subject). Exemplary methods for disrupting the endogenous PGRN or GRN expression are those in which an inhibitory RNA molecule is administered to the subject, or contacted with a population of neurons in the subject or the population of cells to be administered to the subject. The inhibitory RNA molecule may function to disrupt the endogenous PGRN or GRN, for example, act by way of the RNA interference (RNAi) pathway. An inhibitory RNA molecule can decrease the expression level (e.g., protein level or mRNA level) of the endogenous PGRN or GRN. For example, an inhibitory RNA molecule includes a short interfering RNA, short hairpin RNA, and/or a micro RNA that targets a full-length endogenous PGRN or GRN. A siRNA is a double-stranded RNA molecule that typically has a length of about 19-25 base pairs. A shRNA is a RNA molecule including a hairpin turn that decreases expression of target genes via RNAi. shRNAs can be delivered to cells in the form of plasmids, e.g., viral or bacterial vectors, e.g., by transfection, electroporation, or transduction). A micro RNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability or decrease immunogenicity.

In some embodiments, the inhibitory RNA molecule decreases the level and/or activity or function of the endogenous PGRN or GRN. In embodiments, the inhibitory RNA molecule inhibits expression of the endogenous PGRN or GRN. In other embodiments, the inhibitor RNA molecule increases degradation of the endogenous PGRN or GRN and/or decreases the stability of the endogenous PGRN or GRN. The inhibitory RNA molecule can be chemically synthesized or transcribed in vitro.

In some embodiments, the endogenous PGRN or GRN is disrupted in the cells containing the PGRN or the GRN transgene using, for example, the gene editing techniques described herein. In some embodiments, the endogenous PGRN or GRN is globally disrupted in the subject using, for example, the gene editing techniques described herein. In some embodiments, the endogenous PGRN or GRN is disrupted in a population of neurons in the subject using, for example, the gene editing techniques described herein. In some embodiments, disruption of the endogenous PGRN or GRN in the subject, neurons, and/or cells containing the PGRN or the GRN transgene occurs prior to administration of the cells to the subject.

The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology), Humana Press (2010).

### Nuclease-Mediated Gene Regulation

Another useful tool for the disruption and/or integration of target genes into the genome of a cell (e.g., pluripotent cell, ESC, iPSC, multipotent cell, CD34+ cell, HSC, MPC, BLPC, monocyte, macrophage, microglial progenitor cell, or microglial cell) is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and a CRISPR-associated protein (Cas; e.g., Cas9 or Cas12a). This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas nuclease to this site. In this manner, highly site-specific Cas-mediated DNA cleavage can be engendered in a foreign polynucleotide because the interaction that brings Cas within close proximity of the target DNA molecule is governed by RNA: DNA hybridization. As a result, one can theoretically design a CRISPR/Cas system to cleave any target DNA molecule of interest (e.g., the endogenous PGRN or GRN). This technique has been exploited in order to edit eukaryotic genomes (Hwang et al. Nature Biotechnology 31:227 (2013), the disclosure of which is incorporated herein by reference) and can be used as an efficient means of site-specifically editing cell genomes in order to cleave DNA prior to the incorporation of a gene encoding a target gene. The use of CRISPR/Cas to modulate gene expression has been described in, e.g., U.S. Pat. No. 8,697,359, the disclosure of which is incorporated herein by reference. Alternative methods for disruption of a target DNS by site-specifically cleaving genomic DNA prior to the incorporation of a gene of interest in a cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. Nature Reviews Genetics 11:636 (2010); and in Joung et al. Nature Reviews Molecular Cell Biology 14:49 (2013), the disclosure of both of which are incorporated herein by reference. In some embodiments, the endogenous PGRN or GRN may be disrupted in the cells containing the PGRN or the GRN transgene using these gene editing techniques described herein.

### Transposon-Mediated Gene Regulation

In addition to viral vectors, a variety of additional tools have been developed that can be used for the incorporation of exogenous genes into cells (e.g., pluripotent cells, ESCs, iPSCs, multipotent cells, CD34+ cells, HSCs, MPCs, BLPCs, monocytes, macrophages, microglial progenitor cells, or microglia). One such method that can be used for incorporating polynucleotides encoding target genes into cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Transposon systems include the piggybac transposon (described in detail in, e.g., WO 2010/085699) and the sleeping beauty transposon (described in detail in, e.g., US 2005/0112764), the disclosures of each of which are incorporated herein by reference.

### Methods of Diagnosis

Subjects may be diagnosed as having an NCD (e.g., FTLD or NCL) using methods well-known in the art, such as, e.g., the methods described in The Diagnostic and Statistical Manual of Mental Disorders, Fifth Edition and the International Classification of Diseases, 11^{th} Revision. For example, diagnosis of NCDs in a subject may be guided by neuropsychological testing to assess the degree of cognitive impairment in a subject. The subject's cognitive function may be assessed by performing cognitive tests that evaluate performance across one or more cognitive domains including but not limited to complex attention, executive function, learning and memory, language, perceptual-motor function, and social cognition. Comparison of cognitive function in the subject relative to a norm appropriate for the subjects age, medical history, education, socioeconomic status, and lifestyle (e.g., a reference population, such as, e.g., a general population) may be done to determine the diagnosis with respect to an NCD in the subject. The subject may be diagnosed as having a major NCD or a mild NCD. Major NCD is characterized by significant cognitive decline that interferes with personal independence and normal daily functioning and is not due to delirium or other mental disorder. Mild NCD is characterized by moderate cognitive decline that does not interfere with personal independence and normal daily functioning and is not due to delirium or other mental disorder. Major NCD can be characterized by a score obtained on a cognitive test by a subject that is more than two standard deviations away from the mean score of a reference population (e.g., the mean score of a general population) or a score that is in the third percentile of the distribution of scores of the reference population. Mild NCD can be characterized by a score obtained on a cognitive test by a subject that is between one to two standard deviations away from the mean score of a reference population (e.g., the mean score of a general population) or a score that is between the 3^{rd} and 16^{th} percentile of the distribution of scores of the reference population. Non-limiting examples of cognitive tests include Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE). Additionally or alternatively, the use of F18-fluorodeoxyglucose PET scans or MRI scans may be used to determine the presence of neurodegeneration in a subject with an NCD.

Furthermore, the subject may be tested for the presence of biomarkers specific to the particular NCD of interest. For example, a subject may be tested for the presence of biomarkers that indicate that the subject has FTLD, such as, e.g., the presence of tau-positive neuronal and glial inclusions, ub-positive and TDP43-positive but tau-negative inclusions, ub and FUS-positive but tau-negative inclusions, mutations in the PGRN gene disclosed herein and/or mutations on chromosome 17q21 described herein. A subject may also be tested for the presence of lipofuscin inclusions within cells of the body, such as neuronal, liver, spleen, myocardium, and kidney cells, and mutations in one or more CLN genes or the PGRN gene to determine whether the subject has NCL.

### Methods of Treatment

### Selection of Subjects

Subjects that may be treated as described herein are subjects having or at risk of developing an NCD (e.g., FTLD or NCL). The type of FTLD may be PGRN-associated FTLD, including but not limited to behavioral-variant frontotemporal dementia, semantic dementia, and progressive nonfluent aphasia variants of FTLD. The type of NCL may be PGRN-associated NCL, including but not limited to Santavuori-Haltia disease, Jansky-Bielschowsky disease, Batten disease, Kuf's disease, Finnish late infantile NCL, variant late infantile NCL, CLN7 NCL, CLN8 NCL, Turkish late infantile NCL, type 9 NCL, CLN10 NCL, and CLN11 NCL.

Additionally, the type of NCD may be sporadic, NCD caused by an environmental toxin, e.g., herbicides or pesticides, or an NCD associated with a non-PG RN mutation, e.g., a mutation in one or more of the genes associated with the NCD. The compositions and methods described herein can be used to treat subjects with normal PGRN or GRN activity, reduced PGRN or GRN activity, and subjects whose PGRN mutational status and/or PGRN or GRN activity level is unknown. The compositions and methods described herein may also be administered as a preventative treatment to subjects at risk of developing an NCD, e.g., subjects with a PGRN mutation, subjects with reduced PGRN or GRN activity, subjects with a mutation in one or more of the genes associated with an NCD, or subjects exposed to an environmental toxin associated with the NCD. Subjects at risk for an NCD may show early symptoms may not yet be symptomatic when treatment is administered.

In some embodiments, the methods and compositions described herein may be administered to subjects with PGRN mutations that include, for example, frameshift mutations (e.g., p.C31LfsX35, p.C31LfsX35, p.S82VfsX174, p.L271LfsX174, and/or p.T382NfsX32 mutations), missense mutations (p.C521Y, p.A9D, p.P248L, p.R432C, p.C139R, p.C521Y, and/or p.C139R mutations), nonsense mutations (e.g., p.Q125X or p.R493X mutation), insertion mutations (e.g., c.1145insA mutation), and/or transversion mutation (e.g., p.0(IVS1+5G>C mutation). In some embodiments, the methods and compositions described herein may be administered to subjects carrying any other pathogenic mutation in the PGRN gene. For example, pathogenic mutations in the PGRN gene may be any of the mutations discussed in Gijselinck et al., Human Mutation 29: 1373-1386, (2012), the disclosure of which is incorporated herein by reference as it pertains to human PGRN mutations.

### Routes of Administration

The cells and compositions described herein may be administered to a subject with an NCD (e.g., FTLD or NCL) by a variety of routes, such as intracerebroventricularly, intrathecally, intraparenchymally, stereotactically, intravenously, intraosseously, or by means of a bone marrow transplant. In some embodiments, the cells and compositions described herein may be administered to a subject systemically (e.g., intravenously), directly to the central nervous system (CNS) (e.g., intracerebroventricularly, intrathecally, intraparenchymally, or stereotactically), or directly into the bone marrow (e.g., intraosseously). In some embodiments, the cells and compositions described herein are administered to a subject intracerebroventricularly into the cerebral lateral ventricles (a description of this method can be found in Capotondo et al., Science Advances 3:e1701211 (2017), incorporated herein by reference as it pertains to intracerebroventricular injection of hematopoietic stem and progenitor cells into the cerebral lateral ventricles of mouse models). The most suitable route for administration in any given case will depend on the particular cell or composition administered, the subject, pharmaceutical formulation methods, administration methods (e.g., administration time and administration route), the subject's age, body weight, sex, severity of the diseases being treated, the subject's diet, and the subjects excretion rate. Multiple routes of administration may be used to treat a single subject, e.g., intracerebroventricular or stereotactic injection and intravenous injection, intracerebroventricular or stereotactic injection and intraosseous injection, intracerebroventricular or stereotactic injection and bone marrow transplant, intracerebroventricular or stereotactic injection and intraparenchymal injection, intrathecal injection and intravenous injection, intrathecal injection and intraosseous injection, intrathecal injection and bone marrow transplant, intrathecal injection and intraparenchymal injection, intraparenchymal injection and intravenous injection, intraparenchymal injection and intraosseous injection, or intraparenchymal injection and bone marrow transplant. Multiple routes of administration may be used to treat a single subject at one time, or the subject may receive treatment via one route of administration first, and receive treatment via another route of administration during a second appointment, e.g., 1 week later, 2 weeks later, 1 month later, 6 months later, or 1 year later. Cells may be administered to a subject once, or cells may be administered one or more times (e.g., 2-10 times) per week, month, or year to a subject treatment for an NCD.

### Conditioning

Prior to administration of cells (e.g., pluripotent cells, ESCs, iPSCs, multipotent cells, CD34+ cells, HSCs, MPCs, BLPCs, monocytes, macrophages, microglial progenitor cells, or microglia) or compositions, it may be advantageous to deplete or ablate endogenous microglia and/or hematopoietic stem and progenitor cells. Microglia and/or hematopoietic stem and progenitor cells can be ablated through the use of chemical agents (e.g., busulfan, treosulfan, PLX3397, PLX647, PLX5622, or clodronate liposomes), irradiation, or a combination thereof. The agents used for cell ablation may be BBB penetrating (e.g., busulfan) or may lack the ability to cross the BBB (e.g., treosulfan). Exemplary microglia and/or hematopoietic stem and progenitor cells ablating agents are busulfan (Capotondo et al., PNAS 109:15018 (2012), the disclosure of which is incorporated by reference as it pertains to the use of busulfan to ablate microglia), treosulfan, PLX3397, PLX647, PLX5622, or clodronate liposomes. Other agents for the depletion of endogenous microglia and/or hematopoietic stem and progenitor cells include cytotoxins covalently conjugated to antibodies or antigen binding fragments thereof capable of binding antigens expressed by hematopoietic stem cells so as to form an antibody-drug conjugate. Cytotoxins suitable for antibody drug conjugates include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin and derivatives thereof), agents capable of disrupting protein biosynthesis (e.g., agents that exhibit rRNA N-glycosidase activity, such as saporin and ricin A-chain), among others known in the art. Ablation may eliminate all microglia and/or hematopoietic stem and progenitor cells, or it may reduce microglia and/or hematopoietic stem and progenitor cells numbers by at least 5% (e.g., at least 5%, 10%, 20%, 30%, 40%, 50%, 60%, 70%, 80%, 90%, 95%, or more). One or more agents to ablate microglia and/or hematopoietic stem and progenitor cells may be administered at least one week (e.g., 1, 2, 3, 4, 5, or 6 weeks or more) before administration of the cells or compositions described herein. Cells administered in the methods described herein may replace the ablated microglia and/or hematopoietic stem and progenitor cells, and may repopulate the brain following intracerebroventricular, stereotactic, intravenous, or intraosseous injection, or following bone marrow transplant. Cells administered intravenously, intraosseously, or by bone marrow transplant may cross the blood-brain barrier to enter the brain and differentiate into microglia. Cells administered to the brain, e.g., cells administered intracerebroventricularly or stereotactically, can differentiate into microglia in vivo or can be differentiated into microglia ex vivo.

### Stem Cell Rescue

The methods described herein may include administering to a subject a population of cells (e.g., pluripotent cells, ESCs, iPSCs, multipotent cells, CD34+ cells, HSCs, MPCs, BLPCs, monocytes, macrophages, microglial progenitor cells, or microglia). These cells may be cells that have not been modified to express the transgene encoding PGRN or GRN. These cells may have disrupted endogenous PGRN or GRN. The cells may be administered systemically (e.g., intravenously), or by bone marrow transplantation to reconstitute the bone marrow compartment following conditioning as described herein. For example, these cells may migrate to a stem cell niche and increase the quantity of cells of the hematopoietic lineage at such a site by, for example, 1%, 2%, 3%, 4%, 5%, 6%, 7%, 8%, 9%, 10%, 11%, 12%, 13%, 14%, 15%, 16%, 35 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more. Administration may occur prior to or following administration of the composition of the described herein.

### Selection of Donor Cells

In some embodiments, the subject is the donor. In such cases, withdrawn cells (e.g., pluripotent cells, ESCs, iPSCs, multipotent cells, CD34+ cells, HSCs, MPCs, BLPCs, monocytes, macrophages, microglial progenitor cells, or microglia) may be re-infused into the subject following modification (e.g., incorporation of the transgene encoding the PGRN or the GRN, and/or disruption of the endogenous PGRN or GRN), such that the cells may subsequently home to hematopoietic tissue and establish productive hematopoiesis, thereby populating or repopulating a line of cells that is defective or deficient in the subject (e.g., a population of microglia). In this scenario, the transplanted cells are least likely to undergo graft rejection, as the infused cells are derived from the subject and express the same HLA class me and class II antigens as expressed by the subject. Alternatively, the subject and the donor may be distinct. In some embodiments, the subject and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign and are thus less likely to mount an immune response against the transplant. Exemplary HLA-matched donor-recipient pairs are donors and recipients that are genetically related, such as familial donor-recipient pairs (e.g., sibling donor-recipient pairs). In some embodiments, the subject and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

### Pharmaceutical Compositions and Dosing

The number of cells administered to a subject for the treatment of an NCD (e.g., FTLD or NCL, such as, e.g., PGRN-associated FTLD or NCL) as described herein may depend, for example, on the expression level of PGRN or GRN, the subject, pharmaceutical formulation methods, administration methods (e.g., administration time and administration route), the subjects age, body weight, sex, severity of the disease being treated, and whether or not the subject has been treated with agents to ablate endogenous microglia. The number of cells administered may be, for example, from 1×10⁶ cells/kg to 1×10¹² cells/kg, or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more). Cells may be administered in an undifferentiated state, or after partial or complete differentiation into microglia. The number of cells may be administered in any suitable dosage following conditioning. Non-limiting examples of dosages are about 1×10⁵ as cells/kg of recipient to about 1×10⁷ cells/kg (e.g., from about 2×10⁵ as cells/kg to about 9×10⁶ cells/kg, from about 3×10⁵ as cells/kg to about 8×10⁶ cells/kg, from about 4×10⁵ as cells/kg to about 7×10⁶ cells/kg, from about 5×10⁵ as cells/kg to about 6×10⁶ cells/kg, from about 5×10⁵ as cells/kg to about 1×10⁷ cells/kg, from about 6×10⁵ as cells/kg to about 1×10⁷ cells/kg, from about 7×10⁵ as cells/kg to about 1×10⁷ cells/kg, from about 8×10⁵ as cells/kg to about 1×10⁷ cells/kg, from about 9×10⁵ as cells/kg to about 1×10⁷ cells/kg, and from about 1×10⁶ cells/kg to about 1×10⁷ cells/kg). Additional exemplary dosages are from about 1×10¹⁰ cells/kg of recipient to about 1×10¹² cells/kg (e.g., from about 2×10¹⁰ cells/kg to about 9×10¹¹ cells/kg, from about 3×10¹⁰ cells/kg to about 8×10¹¹ cells/kg, from about 4×10¹⁰ cells/kg to about 7×10¹¹ cells/kg, from about 5×10¹⁰ cells/kg to about 6×10¹¹ cells/kg, from about 5×10¹⁰ cells/kg to about 1×10¹² cells/kg, from about 6×10¹⁰ cells/kg to about 1×10¹² cells/kg, from about 7×10¹⁰ cells/kg to about 1×10¹² cells/kg, from about 8×10¹⁰ cells/kg to about 1×10¹² cells/kg, from about 9×10¹⁰ cells/kg to about 1×10¹² cells/kg, and from about 1×10¹¹ cells/kg to about 1×10¹² cells/kg), among others.

The cells and compositions described herein can be administered in an amount sufficient to improve one or more pathological features in the NCD. Administration of the cells or compositions described herein may increase the quantity of M2 microglia in the brain of the subject relative to the quantity of M1 microglia in the brain of the subject, decrease the level of pro-inflammatory cytokines in the brain of the subject, increase the level of anti-inflammatory cytokines in the brain of the subject, improve the cognitive performance of the subject, improve the motor function of the subject, reduce α-synuclein protein levels, tau-positive neuronal inclusion levels, TAR DNA-binding protein 43 (TDP-43)-positive inclusion levels, fused in sarcoma (FUS)-positive inclusion levels, and/or ubiquitin-positive inclusion levels or aggregation thereof in the subject, reduce loss of brain tissue in the subject, improve vision, improve language skills, and/or reduce the severity or frequency of occurrence of epileptic seizures. The numbers of M1 and M2 microglia may be assessed using ELISAs to compare the level of cytokines, chemokines, and other pro- and anti-inflammatory mediators in the cerebrospinal fluid (CSF) of subjects before and after treatment, by using PET imaging to view translocator protein (TSPO), a protein highly expressed in classically activated M1 microglia, before and after treatment, e.g., using TSPO radioligand ¹¹C-(R)PK11195, or by analyzing the levels of M1- and M2-associated genes and proteins in a tissue sample using standard techniques, e.g., western blot analysis, immunohistochemical analyses, or quantitative RT-PCR. Cognition and motor function can be assessed using standard neurological tests before and after treatment, and monomeric and oligomeric α-synuclein can be detected in plasma and CSF using ELISA. Neurodegeneration can be assessed using F18-fluorodeoxyglucose PET scans or MRI scans. The subject may be evaluated 1 month, 2 months, 3 months, 4 months, 5 months, 6 months or more following administration of the population of cells depending on the route of administration used for treatment. Depending on the outcome of the evaluation, the subject may receive additional treatments.

### Kits

The compositions described herein can be provided in a kit for use in treating an NCD (e.g., FTLD or NCL). Compositions may include host cells described herein (e.g., pluripotent cells, ESCs, iPSCs, multipotent cells, CD34+ cells, HSCs, MPCs, BLPCs, monocytes, macrophages, microglial progenitor cells, or microglia) that contain a transgene (e.g., a transgene capable of expression in macrophages or microglia) encoding a PGRN or a GRN, and, optionally, may have disrupted endogenous PGRN or GRN. Cells may be cryopreserved, e.g., in dimethyl sulfoxide (DMSO), glycerol, or another cryoprotectant. The kit can include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Generation of a Cell Containing a Transgene Encoding Progranulin or Granulin

An exemplary method for making cells (e.g., pluripotent cells, embryonic stem cells (ESCs), induced pluripotent stem cells (ISPCs), multipotent cells, CD34+ cells, hematopoietic stem cells (HSCs), myeloid precursor cells (MPCs), blood lineage progenitor cells, monocytes, macrophages, microglial progenitor cells, or microglia) containing a transgene encoding a progranulin (PGRN) or a granulin (GRN) for use in the compositions and methods described herein is by way of transduction. Retroviral vectors (e.g., a lentiviral vector, alpharetroviral vector, or gammaretroviral vector) containing a microglia-specific promoter, such as the CD68 promoter, and the polynucleotide encoding the PGRN or the GRN can be engineered using standard techniques known in the art. After the retroviral vector is engineered, the retrovirus can be used to transduce cells to generate a population of cells that express the PGRN or the GRN.

Additional exemplary methods for making cells containing a transgene encoding the PGRN or the GRN for use in the compositions and methods described herein is transfection. Using molecular biology techniques known in the art, plasmid DNA containing a promoter, such as a microglia-specific promoter, (e.g., the CD68 promoter), and the polynucleotide encoding the PGRN or the GRN can be produced. For example, the PGRN gene may be amplified from a human cell line using PCR-based techniques known in the art, or a PGRN or a GRN gene may be synthesized, for example, using solid-phase polynucleotide synthesis procedures. The PGRN or the GRN gene and promoter can then be ligated into a plasmid of interest, for example, using suitable restriction endonuclease-mediated cleavage and ligation protocols. After the plasmid DNA is engineered, the plasmid can be used to transfect the cells using, for example, electroporation or another transfection technique described herein to generate a population of cells that express the PGRN or the GRN. In both exemplary methods described herein, the PGRN or the GRN may be expressed as a PGRN or a GRN fusion protein. The PGRN or the GRN fusion protein may contain a peptide sequence containing the LDLRf Rb domain of ApoE to allow for the penetrance of the PGRN or the GRN fusion protein across the blood-brain barrier. Alternatively, the fusion protein may contain PGRN or GRN and a glycosylation independent lysosomal targeting (GILT) tag. Exemplary GILT tags are muteins derived from human insulin-like growth factor II (IGF-II) having an amino acid sequence that is at least 70% identical to the amino acid sequence of mature human IGF-II. These IGF-II muteins have diminished binding activity for the insulin receptor relative to the affinity of naturally-occurring human IGF-II for the insulin receptor, are resistant to furin cleavage, and bind to the human cation-independent mannose-6-phosphate receptor in a mannose-6-phosphate-independent manner. The GILT tag facilitates delivery of the secreted GBA fusion protein to the lysosome.

### Example 2. Administration of a Population of Cells Containing a Transgene Encoding Progranulin or Granulin to a Subject Suffering from a Neurocognitive Disorder

According to the methods disclosed herein, a physician of skill in the art can treat a subject, such as a human subject, so as to reduce or alleviate symptoms of a neurocognitive disorder (NCD), such as, e.g., frontotemporal lobar degeneration (FTLD) or neuronal ceroid lipofuscinosis (NCL). To this end, a physician of skill in the art can administer to the human subject a population of cells (e.g., pluripotent cells, ESCs, iPSCs, multipotent cells, CD34+ cells, HSCs, MPCs, BLPCs, monocytes, macrophages, microglial progenitor cells, or microglia) containing a transgene (e.g., a transgene capable of expression in macrophages or microglia) encoding the PGRN or the GRN. The cells can be transduced or transfected ex vivo to express the PGRN or the GRN using techniques described herein or known in the art. The population of cells containing a transgene encoding the PGRN or the GRN may be administered to the subject, for example, systemically (e.g., intravenously), directly to the CNS (e.g., intracerebroventricularly or stereotactically), or directly into the bone marrow (e.g., intraosseously), to treat the NCD. The cells can also be administered to the subject by multiple routes of administration, for example, intravenously and intracerebroventricularly. The cells are administered in a therapeutically effective amount, such as from 1×10⁶ cells/kg to 1×10¹² cells/kg or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more).

Before the population of cells is administered to the subject, one or more agents may be administered to the subject to ablate the subject's endogenous microglia and/or hematopoietic stem and progenitor cells, for example, busulfan, treosulfan, PLX3397, PLX647, PLX5622, and/or clodronate liposomes. Other methods of cell ablation well known in the art, such as irradiation, may be used alone or in combination with one or more of the aforementioned agents to ablate the subject's microglia and/or hematopoietic stem and progenitor cells. These agents and/or treatments may ablate endogenous microglia and/or hematopoietic stem and progenitor cells by at least 5% (e.g., at least 5%, 10%, 15%, 20%, 25%, 30%, 35%, 40%, 45%, 50%, 60%, 70%, 80%, 90%, 99%, or more), as assessed by PET imaging techniques known in the art. If the population of cells is administered to the subject after microglial ablation, the cells can repopulate the brain, differentiating into microglia. The population of cells can be administered to the subject from, for example, 1 week to 1 month (e.g., 1 week, 2 weeks, 3 weeks, 4, weeks) or more after microglial ablation.

Following ablation of the subject's endogenous microglia and/or hematopoietic stem and progenitor cells, a population of cells may be administered to the subject systemically (e.g., intravenously), or by bone marrow transplantation to reconstitute the bone marrow compartment. The number of cells may be administered in any suitable dosage following conditioning. Non-limiting examples of dosages are about 1×10⁵ as cells/kg of recipient to about 1×10⁷ cells/kg (e.g., from about 2×10⁵ as cells/kg to about 9×10⁶ cells/kg, from about 3×10⁵ as cells/kg to about 8×10⁶ cells/kg, from about 4×10⁵ as cells/kg to about 7×10⁶ cells/kg, from about 5×10⁵ as cells/kg to about 6×10⁶ cells/kg, from about 5×10⁵ as cells/kg to about 1×10⁷ cells/kg, from about 6×10⁵ as cells/kg to about 1×10⁷ cells/kg, from about 7×10⁵ as cells/kg to about 1×10⁷ cells/kg, from about 8×10⁵ as cells/kg to about 1×10⁷ cells/kg, from about 9×10⁵ as cells/kg to about 1×10⁷ cells/kg, or from about 1×10⁶ cells/kg to about 1×10⁷ cells/kg, among others). Administration may occur prior to or following administration of the cells containing the transgene encoding the PGRN or the GRN. The population of cells can be administered to the subject in an amount sufficient to treat one or more of the pathological features of the NCD. For example, the population of cells containing the transgene encoding the PGRN or the GRN can be administered in an amount sufficient to increase the quantity of M2 microglia in the brain of the subject relative to the quantity of M1 microglia in the brain of the subject. The relative increase can be measured using conventional techniques known in the art, such as by performing an ELISA on subject CSF before and after treatment to assess the level of pro-inflammatory and anti-inflammatory cytokines secreted by M1 and M2 microglia at both time points. A standard neurological examination can also be performed by the physician before and after treatment to evaluate changes in cognitive performance and motor function. The subject may be evaluated, for example, 1 month, 2 months, 3 months, 4 months, 5 months, 6 months or more following administration of the population of cells depending on the route of administration used for treatment. A finding of reduced pro-inflammatory cytokines, increased anti-inflammatory cytokines, improved cognitive or motor function, improved vision, improved language skills, and/or reduced the severity or frequency of occurrence of epileptic seizures following administration of a population of cells containing a transgene encoding the PGRN or the GRN provides an indication that the treatment has successfully treated the NCD.

### Example 3. Disruption of Endogenous Progranulin or Granulin in Cells Prior to Administration to a Subject Suffering from a Neurocognitive Disorder

In any of the methods disclosed herein, the cells (e.g., pluripotent cells, ESCs, iPSCs, multipotent cells, CD34+ cells, HSCs, MPCs, BLPCs, monocytes, macrophages, microglial progenitor cells, or microglia) to be administered may be treated to disrupt the endogenous PGRN or GRN prior to administration to the subject. An exemplary method of disrupting the endogenous PGRN or GRN in cells is using a CRISPR/Cas system (e.g., CRISPR/Cas9 or CRISPR/Cas12a) with a PGRN-specific guide RNA (gRNA) to induce one or more double-strand breaks (DSB). Following non-homologous end joining (NHEJ) to repair the DSB, the presence of newly-formed indel mutations will result in the endogenous PGRN or GRN disruption. Alternative methods for disruption of endogenous PGRN or GRN by site-specifically cleaving genomic DNA prior to the incorporation of the PGRN or the GRN transgene in a pluripotent stem cell include the use of zinc finger nucleases (ZFNs) and transcription activator-like effector nucleases (TALENs). Unlike the CRISPR/Cas system, these enzymes do not contain a guiding polynucleotide to localize to a specific target sequence, but instead rely on internal DNA biding domains within the enzymes to mediate target specificity. In exemplary embodiments, the cell is manipulated ex vivo by the nuclease to decrease or reduce the expression of the endogenous PGRN or GRN by 10% or more (e.g., 10%, 20%, 30%, 40%, 50%, 60%, 70%, 80%, 90%, 95% or more).

### Example 4. Generation of Mammalian Cell Lines Expressing Progranulin

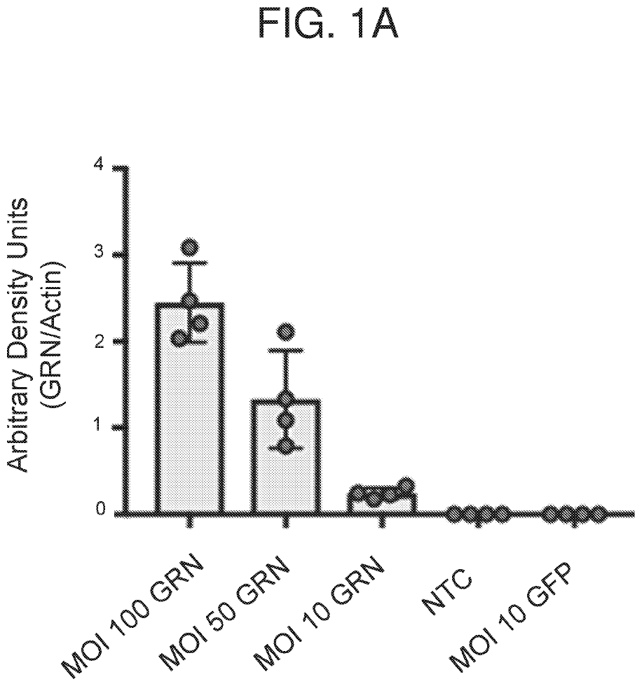

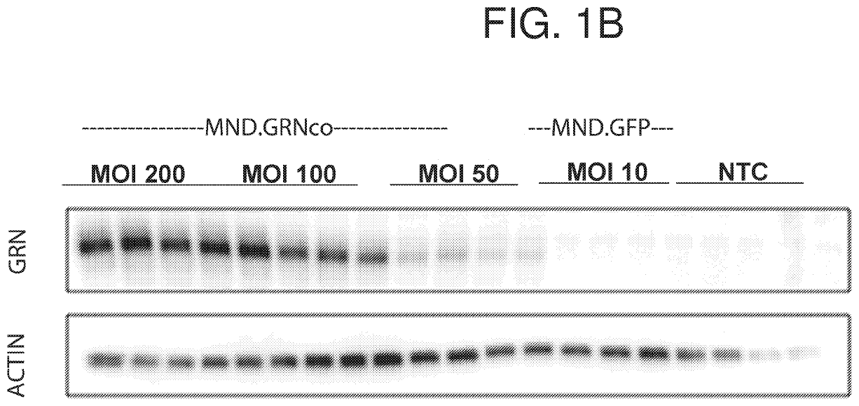

To assess the ability of lentivirally-encoded, codon-optimized PGRN transgenes to stably express in mammalian cell lines, human and murine cells were transduced in vitro. In a first experiment, human 239T cells were transduced with a lentiviral vector containing a transgene encoding a human PGRN protein (MND.GRN) or green fluorescent protein (GFP; MND.GFP) at a multiplicity of infection (MOI) of 10, 50, 100, or 200. A separate set of control cells were not transduced (NTC). Densitometry was used to quantify PGRN levels over actin (FIG. 1A). Western blots were performed using an antibody raised against human PGRN protein, demonstrating stable expression of human PGRN in human cells, with the highest expression observed at MOI 200 (FIG. 1B).

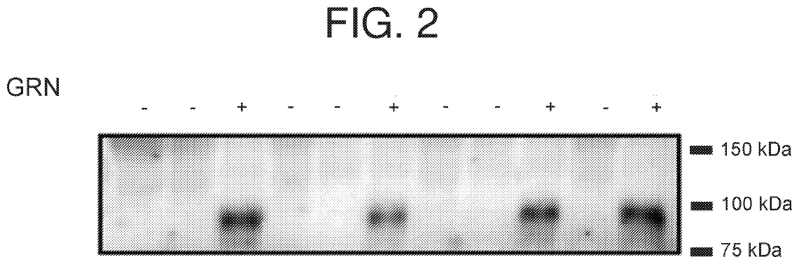

In a separate experiment, murine lineage negative (Lin−) cells were transduced with a lentiviral vector containing a transgene encoding human PGRN protein (i.e., a MND.GRN vector). Conditioned media generated from Lin− mouse cells non-transduced or transduced with MND.GRN lentiviral vector was analyzed using Western blot with an antibody raised against human PGRN protein, showing release of human PGRN protein into the growth media by the transduced cells (FIG. 2).

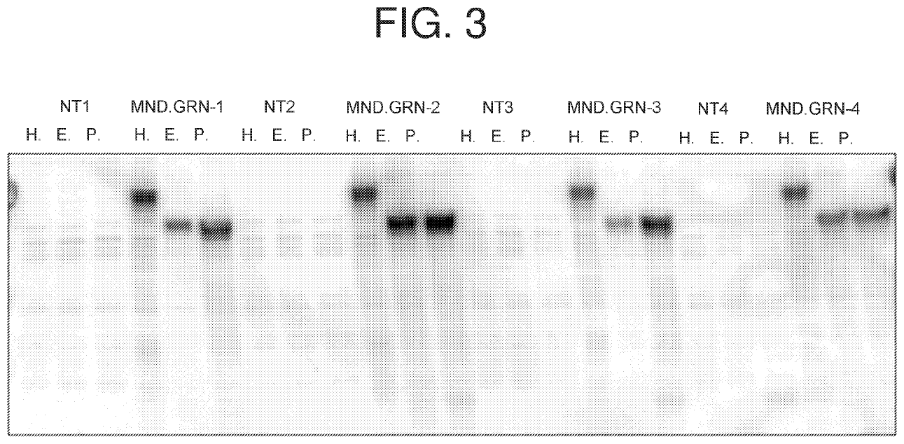

In another experiment, human 239T cells were transduced with a lentiviral vector containing a transgene encoding a human PGRN protein in four independent rounds of transduction. Cell lysates were generated from 239T non-transduced cells or cell lines transduced with a lentiviral vector encoding human PG RN. Cell lysates were then enzymatically digested with EndoH or PNGase enzymes, or heated, and analyzed using Western blot with an antibody raised against human PGRN protein (FIG. 3). Enzymatic digestion by EndoH and PNGase indicate that the human PGRN protein produced by the transduced cells is N-linked glycosylated.

Combined together, the above results show that lentivirally-mediated transduction of human and murine cells with transgenes encoding a human PGRN protein achieves stable PGRN expression in cells in which PGRN expression is otherwise absent. Transduction of murine primary Lin− cells with lentivirally-encoded PGRN results in the release of PGRN protein into the growth media. Furthermore, the PGRN protein produced by the lentiviral vector described above is N-linked glycosylated. These findings demonstrate that lentiviral transduction with the PGRN-encoding vector described above increases PGRN levels and enables the release of PGRN by hematopoietic cells, thereby suggesting a potential therapeutic approach for diseases caused by or linked to mutations in the PGRN gene.

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

1. A method of treating a subject diagnosed as having a neurocognitive disorder (NCD), the method comprising administering to the subject a composition comprising a population of cells comprising a transgene encoding a progranulin (PGRN) or a granulin (GRN).

2. The method of claim 1, wherein the NCD is a major NCD.

3. The method of claim 2, wherein the major NCD interferes with the subject's independence and/or normal daily functioning.

4. The method of claim 2 or 3, wherein the major NCD is associated with a score obtained by the subject on a cognitive test that is at least two standard deviations away from the mean score of a reference population.

5. The method of claim 1, wherein the NCD is a mild NCD.

6. The method of claim 5, wherein the mild NCD does not interfere with the subject's independence and/or normal daily functioning.

7. The method of claim 5 or 6, wherein the mild NCD is associated with a score obtained by the subject on a cognitive test that is between one to two standard deviations away from the mean score of a reference population.

8. The method of claim 4 or 7, wherein the reference population is a general population.

9. The method of claim 4, 7, or 8, wherein the cognitive test is selected from the group consisting of Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE).

10. The method of any one of claims 1-9, wherein the NCD is associated with impairment in one or more of complex attention, executive function, learning and memory, language, perceptual-motor function, and social cognition.

11. The method of any one of claims 1-10, wherein the NCD is not due to delirium or other mental disorder.

12. The method of any one of claims 1-11, wherein the NCD is a frontotemporal NCD.

13. The method of claim 12, wherein the frontotemporal NCD is frontotemporal lobar degeneration (FTLD).

14. The method of any one of claims 1-11, wherein the NCD is due to a lysosomal disease.

15. The method of claim 14, wherein the lysosomal disease is neuronal ceroid lipofuscinosis (NCL).

16. The method of any one of claims 1-15, wherein the PGRN or the GRN comprises a secretory signal peptide.

17. The method of claim 16, wherein the secretory signal peptide is a PGRN secretory signal peptide.

18. The method of any one of claims 1-17, wherein the cells comprise a transgene encoding the PGRN.

19. The method of claim 18, wherein the PGRN comprises at least 2 GRN domains, optionally wherein the PGRN comprises at least 3 GRN domains, optionally wherein the PGRN comprises at least 4 GRN domains, optionally wherein the PGRN comprises at least 5 GRN domains, optionally wherein the PGRN comprises at least 6 GRN domains, optionally wherein the PGRN comprises at least 7 GRN domains, or optionally wherein the PGRN comprises at least 8 GRN domains.

20. The method of any one of claims 1-19, wherein the PGRN comprises from 2 to 16 GRN domains, optionally wherein the PGRN comprises from 2 to 12 GRN domains, optionally wherein the PGRN comprises from 2 to 8 GRN domains, optionally wherein the PGRN comprises from 2 to 4 GRN domains, or optionally wherein the PGRN comprises 2 GRN domains.

21. The method of any one of claims 1-20, wherein the PGRN comprises a para-GRN domain having an amino acid sequence that is at least 85% identical to the amino acid sequence of SEQ ID NO. 2, optionally wherein the para-GRN domain has an amino acid sequence that is at least 90% identical to the amino acid sequence of SEQ ID NO. 2, optionally wherein the para-GRN domain has an amino acid sequence that is at least 95% identical to the amino acid sequence of SEQ ID NO. 2, or optionally wherein the para-GRN domain has an amino acid sequence of SEQ ID NO. 2.

22. The method of any one of claims 1-21, wherein the PGRN comprises a GRN-1 domain having an amino acid sequence that is at least 85% identical to the amino acid sequence of SEQ ID NO. 3, optionally wherein the GRN-1 domain has an amino acid sequence that is at least 90% identical to the amino acid sequence of SEQ ID NO. 3, optionally wherein the GRN-1 domain has an amino acid sequence that is at least 95% identical to the amino acid sequence of SEQ ID NO. 3, or optionally wherein the GRN-1 domain has the amino acid sequence of SEQ ID NO. 3.

23. The method of any one of claims 1-22, wherein the PGRN comprises a GRN-2 domain having an amino acid sequence that is at least 85% identical to the amino acid sequence of SEQ ID NO. 4, optionally wherein the GRN-2 domain has an amino acid sequence that is at least 90% identical to the amino acid sequence of SEQ ID NO. 4, optionally wherein the GRN-2 domain has an amino acid sequence that is at least 95% identical to the amino acid sequence of SEQ ID NO. 4, or optionally wherein the GRN-2 domain has an amino acid sequence of SEQ ID NO. 4.

24. The method of any one of claims 1-23, wherein the PGRN comprises a GRN-3 domain having an amino acid sequence that is at least 85% identical to the amino acid sequence of SEQ ID NO. 5, optionally wherein the GRN-3 domain has an amino acid sequence that is at least 90% identical to the amino acid sequence of SEQ ID NO. 5, optionally wherein the GRN-3 domain has an amino acid sequence that is at least 95% identical to the amino acid sequence of SEQ ID NO. 5, or optionally wherein the GRN-3 domain has an amino acid sequence of SEQ ID NO. 5.

25. The method of any one of claims 1-24, wherein the PGRN comprises a GRN-4 domain having an amino acid sequence that is at least 85% identical to the amino acid sequence of SEQ ID NO. 6, optionally wherein GRN-4 domain has an amino acid sequence that is at least 90% identical to the amino acid sequence of SEQ ID NO. 6, optionally wherein the GRN-4 domain has an amino acid sequence that is at least 95% identical to the amino acid sequence of SEQ ID NO. 6, or optionally wherein the GRN-4 domain has an amino acid sequence of SEQ ID NO. 6.

26. The method of any one of claims 1-25, wherein the PGRN comprises a GRN-5 domain having an amino acid sequence that is at least 85% identical to the amino acid sequence of SEQ ID NO. 7, optionally wherein the GRN-5 domain has an amino acid sequence that is at least 90% identical to the amino acid sequence of SEQ ID NO. 7, optionally wherein the GRN-5 domain has an amino acid sequence that is at least 95% identical to the amino acid sequence of SEQ ID NO. 7, or optionally wherein the GRN-5 domain has an amino acid sequence of SEQ ID NO. 7.

27. The method of any one of claims 1-26, wherein the PGRN comprises a GRN-6 domain having an amino acid sequence that is at least 85% identical to the amino acid sequence of SEQ ID NO. 8, optionally wherein the GRN-6 domain has an amino acid sequence that is at least 90% identical to the amino acid sequence of SEQ ID NO. 8, optionally wherein the GRN-6 domain has an amino acid sequence that is at least 95% identical to the amino acid sequence of SEQ ID NO. 8, or optionally wherein the GRN-6 domain has an amino acid sequence of SEQ ID NO. 8.

28. The method of any one of claims 1-27, wherein the PGRN comprises a GRN-7 domain having an amino acid sequence that is at least 85% identical to the amino acid sequence of SEQ ID NO. 9, optionally wherein the GRN-7 domain has an amino acid sequence that is at least 90% identical to the amino acid sequence of SEQ ID NO. 9, optionally wherein the GRN-7 domain has an amino acid sequence that is at least 95% identical to the amino acid sequence of SEQ ID NO. 9, or optionally wherein the GRN-7 domain has an amino acid sequence of SEQ ID NO. 9.

29. The method of any one of claims 1-28, wherein the PGRN has an amino acid sequence that is at least 85% identical to the amino acid sequence of SEQ ID NO. 1, optionally wherein the PGRN has an amino acid sequence that is at least 90% identical to the amino acid sequence of SEQ ID NO. 1, optionally wherein the PGRN has an amino acid sequence that is at least 95% identical to the amino acid sequence of SEQ ID NO. 1, or optionally wherein the PGRN has an amino acid sequence of SEQ ID NO. 1.

30. The method of any one of claims 1-29, wherein the PGRN is a full-length PGRN.

31. The method of any one of claims 1-30, wherein the cells comprise a transgene encoding the GRN.

32. The method of any one of claims 1-31, wherein the GRN is a para-GRN domain having an amino acid sequence that is at least 85% identical to the amino acid sequence of SEQ ID NO. 2, optionally wherein the para-GRN domain has an amino acid sequence that is at least 90% identical to the amino acid sequence of SEQ ID NO. 2, optionally wherein the para-GRN domain has an amino acid sequence that is at least 95% identical to the amino acid sequence of SEQ ID NO. 2, or optionally wherein the para-GRN domain has an amino acid sequence of SEQ ID NO. 2.

33. The method of any one of claims 1-32, wherein the GRN is a GRN-1 domain having an amino acid sequence that is at least 85% identical to the amino acid sequence of SEQ ID NO. 3, optionally wherein the GRN-1 domain has an amino acid sequence that is at least 90% identical to the amino acid sequence of SEQ ID NO. 3, optionally wherein the GRN-1 domain has an amino acid sequence that is at least 95% identical to the amino acid sequence of SEQ ID NO. 3, or optionally wherein the GRN-1 domain has an amino acid sequence of SEQ ID NO. 3.

34. The method of any one of claims 1-33, wherein the GRN is a GRN-2 domain having an amino acid sequence that is at least 85% identical to the amino acid sequence of SEQ ID NO. 4, optionally wherein the GRN-2 domain has an amino acid sequence that is at least 90% identical to the amino acid sequence of SEQ ID NO. 4, optionally wherein the GRN-2 domain has an amino acid sequence that is at least 95% identical to the amino acid sequence of SEQ ID NO. 4, or optionally wherein the GRN-2 domain has an amino acid sequence of SEQ ID NO. 4.

35. The method of any one of claims 1-34, wherein the GRN is a GRN-3 domain having an amino acid sequence that is at least 85% identical to the amino acid sequence of SEQ ID NO. 5, optionally wherein the GRN-3 domain has an amino acid sequence that is at least 90% identical to the amino acid sequence of SEQ ID NO. 5, optionally wherein the GRN-3 domain has an amino acid sequence that is at least 95% identical to the amino acid sequence of SEQ ID NO. 5, or optionally wherein the GRN-3 domain has an amino acid sequence of SEQ ID NO. 5.

36. The method of any one of claims 1-35, wherein the GRN is a GRN-4 domain having an amino acid sequence that is at least 85% identical to the amino acid sequence of SEQ ID NO. 6, optionally wherein the GRN-4 domain has an amino acid sequence that is at least 90% identical to the amino acid sequence of SEQ ID NO. 6, optionally wherein the GRN-4 domain has an amino acid sequence that is at least 95% identical to the amino acid sequence of SEQ ID NO. 6, or optionally wherein the GRN-4 domain has an amino acid sequence of SEQ ID NO. 6.

37. The method of any one of claims 1-36, wherein the GRN is a GRN-5 domain having an amino acid sequence that is at least 85% identical to the amino acid sequence of SEQ ID NO. 7, optionally wherein the GRN-5 domain has an amino acid sequence that is at least 90% identical to the amino acid sequence of SEQ ID NO. 7, optionally wherein the GRN-5 domain has an amino acid sequence that is at least 95% identical to the amino acid sequence of SEQ ID NO. 7, or optionally wherein the GRN-5 domain has an amino acid sequence of SEQ ID NO. 7.

38. The method of any one of claims 1-37, wherein the GRN is a GRN-6 domain having an amino acid sequence that is at least 85% identical to the amino acid sequence of SEQ ID NO. 8, optionally wherein the GRN-6 domain has an amino acid sequence that is at least 90% identical to the amino acid sequence of SEQ ID NO. 8, optionally wherein the GRN-6 domain has an amino acid sequence that is at least 95% identical to the amino acid sequence of SEQ ID NO. 8, or optionally wherein the GRN-6 domain has an amino acid sequence of SEQ ID NO. 8.

39. The method of any one of claims 1-38, wherein the GRN is a GRN-7 domain having an amino acid sequence that is at least 85% identical to the amino acid sequence of SEQ ID NO. 9. optionally wherein the GRN-7 domain has an amino acid sequence that is at least 90% identical to the amino acid sequence of SEQ ID NO. 9, optionally wherein the GRN-7 domain has an amino acid sequence that is at least 95% identical to the amino acid sequence of SEQ ID NO. 9, or optionally wherein the GRN-7 domain has an amino acid sequence of SEQ ID NO. 9.

40. The method of any one of claims 1-39, wherein the GRN comprises a full-length GRN.

41. The method of any one of claims 1-40, wherein the cells comprise a PGRN transgene having at least 85% sequence identity to the nucleic acid sequence of SEQ ID NO. 10, optionally wherein the cells comprise a PGRN transgene having at least 90% sequence identity to the nucleic acid sequence of SEQ ID NO. 10, optionally wherein the cells comprise a PGRN transgene having at least 95% sequence identity to the nucleic acid sequence of SEQ ID NO. 10, optionally wherein the cells comprise a PGRN transgene having the nucleic acid sequence of SEQ ID NO. 10.

42. The method of any one of claims 1-40, wherein the cells comprise a codon-optimized PGRN transgene.

43. The method of claim 42, wherein the codon-optimized transgene has at least 85% sequence identity to the nucleic acid sequence of SEQ ID NO. 19, optionally, wherein the codon-optimized transgene has at least 90% sequence identity to the nucleic acid sequence of SEQ ID NO. 19, optionally, wherein the codon-optimized transgene has at least 95% sequence identity to the nucleic acid sequence of SEQ ID NO. 19, or optionally, wherein the codon-optimized transgene has the nucleic acid sequence of SEQ ID NO. 19.

44. The method of any one of claims 1-43, wherein the PGRN or the GRN is a PGRN or a GRN fusion protein.

45. The method of claim 44, wherein the PGRN or the GRN fusion protein comprises a receptor-binding (Rb) domain of apolipoprotein E (ApoE).

46. The method of claim 45, wherein the Rb domain comprises a portion of ApoE having the amino acid sequence of residues 25-185, 50-180, 75-175, 100-170, 125-160, or 130-150 of SEQ ID NO. 11.

47. The method of claim 45 or 46, wherein the Rb domain comprises a region having at least 70% sequence identity to the amino acid sequence of residues 159-167 of SEQ ID NO. 11.

48. The method of claim 44, wherein the PGRN or the GRN fusion protein comprises PGRN or GRN and a glycosylation independent lysosomal targeting (GILT) tag.

49. The method of claim 48, wherein the GILT tag comprises a human IGF-II mutein having an amino acid sequence that is at least 70% identical to the amino acid sequence of mature human IGF-II (SEQ ID NO. 12), and having diminished binding affinity for the insulin receptor relative to the affinity of naturally-occurring human IGF-II for the insulin receptor, wherein the IGF-II mutein is resistant to furin cleavage and binds to the human cation-independent mannose-6-phosphate receptor in a mannose-6-phosphate-independent manner.

50. The method of claim 49, wherein the IGF-II mutein comprises a mutation within a region corresponding to amino acids 30-40 of SEQ ID NO. 12, and wherein the mutation abolishes at least one furin protease cleavage site.

51. The method of claim 50, wherein the mutation is an amino acid substitution, deletion, and/or insertion.

52. The method of claim 51, wherein the mutation is a Lys or Ala amino acid substitution at a position corresponding to Arg37 or Arg40 of SEQ ID NO. 12.

53. The method of claim 51, wherein the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO. 12, and combinations thereof.

54. The method of any one of claims 48-53, wherein the GILT tag has an amino acid sequence that is at least 70% identical to the amino acid sequence of SEQ ID NO. 13, optionally wherein the GILT tag has an amino acid sequence that is at least 80% identical to the amino acid sequence of SEQ ID NO. 13, optionally wherein the GILT tag has an amino acid sequence that is at least 90% identical to the amino acid sequence of SEQ ID NO. 13, optionally wherein the GILT tag has the amino acid sequence of SEQ ID NO. 13.

55. The method of any one of claims 48-53, wherein the GILT tag has an amino acid sequence that is at least 70% identical to the amino acid sequence of SEQ ID NO. 14, optionally wherein the GILT tag has an amino acid sequence that is at least 80% identical to the amino acid sequence of SEQ ID NO. 14, optionally wherein the GILT tag has an amino acid sequence that is at least 90% identical to the amino acid sequence of SEQ ID NO. 14, optionally wherein the GILT tag has the amino acid sequence of SEQ ID NO. 14.

56. The method of any one of claims 48-53, wherein the GILT tag has an amino acid sequence that is at least 70% identical to the amino acid sequence of SEQ ID NO. 15, optionally wherein the GILT tag has an amino acid sequence that is at least 80% identical to the amino acid sequence of SEQ ID NO. 15, optionally wherein the GILT tag has an amino acid sequence that is at least 90% identical to the amino acid sequence of SEQ ID NO. 15, optionally wherein the GILT tag has the amino acid sequence of SEQ ID NO. 15.

57. The method of any one of claims 48-53, wherein the GILT tag is encoded by a polynucleotide having a nucleic acid sequence that is at least 85% identical to the nucleic acid sequence of SEQ ID NO. 16, optionally wherein the GILT tag is encoded by a polynucleotide having a nucleic acid sequence that is at least 90% identical to the nucleic acid sequence of SEQ ID NO. 16, optionally wherein the GILT tag is encoded by a polynucleotide having a nucleic acid sequence that is at least 95% identical to the nucleic acid sequence of SEQ ID NO. 16, optionally wherein the GILT tag is encoded by a polynucleotide having the nucleic acid sequence of SEQ ID NO. 16.

58. The method of any one of claims 48-53, wherein the GILT tag is encoded by a polynucleotide having a nucleic acid sequence that is at least 85% identical to the nucleic acid sequence of SEQ ID NO. 17, optionally wherein the GILT tag is encoded by a polynucleotide having a nucleic acid sequence that is at least 90% identical to the nucleic acid sequence of SEQ ID NO. 17, optionally wherein the GILT tag is encoded by a polynucleotide having a nucleic acid sequence that is at least 95% identical to the nucleic acid sequence of SEQ ID NO. 17, optionally wherein the GILT tag is encoded by a polynucleotide having the nucleic acid sequence of SEQ ID NO. 17.

59. The method of any one of claims 48-53, wherein the GILT tag is encoded by a polynucleotide having a nucleic acid sequence that is at least 85% identical to the nucleic acid sequence of SEQ ID NO. 18, optionally wherein the GILT tag is encoded by a polynucleotide having a nucleic acid sequence that is at least 90% identical to the nucleic acid sequence of SEQ ID NO. 18, optionally wherein the GILT tag is encoded by a polynucleotide having a nucleic acid sequence that is at least 95% identical to the nucleic acid sequence of SEQ ID NO. 18, optionally wherein the GILT tag is encoded by a polynucleotide having the nucleic acid sequence of SEQ ID NO. 18.

60. The method of any one of claims 1-59, wherein the transgene encoding the PGRN or the GRN further comprises a micro RNA (miRNA)-126 (miR-126) targeting sequence in the 3′-UTR.

61. The method of any one of claims 1-60, wherein upon administration of the composition to the subject, the PGRN or the GRN penetrates the blood-brain barrier in the subject.

62. The method of any one of claims 13-61, wherein the FTLD or NCL is PGRN-associated FTLD or NCL.

63. The method of claim 62, wherein the PGRN-associated FTLD is the behavioral-variant frontotemporal dementia variant of FTLD.

64. The method of claim 62, wherein the PGRN-associated FTLD is the semantic dementia variant of FTLD.

65. The method of claim 62, wherein the PGRN-associated FTLD is the progressive nonfluent aphasia variant of FTLD.

66. The method of claim 62, wherein the PGRN-associated NCL is Batten disease.

67. The method of any one of claims 1-54, wherein the cells are pluripotent cells or multipotent cells.

68. The method of claim 67, wherein the multipotent cells are CD34+ cells.

69. The method of claim 68, wherein the CD34+ cells are hematopoietic stem cells (HSCs) or myeloid progenitor cells (MPCs).

70. The method of claim 67, wherein the pluripotent cells are embryonic stem cells (ESCs) or induced pluripotent stem cells (iPSCs),

71. The method of any one of claims 1-66, wherein the cells are blood lineage progenitor cells (BLPCs), microglial progenitor cells, monocytes, macrophages, or microglia.

72. The method of claim 71, wherein the BLPCs are monocytes.

73. The method of any one of claims 1-72, wherein a population of endogenous microglia in the subject has been ablated prior to administration of the composition.

74. The method of any one of claims 1-72, the method comprising ablating a population of endogenous microglia in the subject prior to administering the composition to the subject.

75. The method of claim 72 or 73 wherein the microglia are ablated using an agent selected from the group consisting of busulfan, PLX3397, PLX647, PLX5622, treosulfan, and clodronate liposomes, by radiation therapy, or a combination thereof.

76. The method of any one of claims 1-75, wherein the composition is administered to the subject by way of systemic administration, by way of direct administration to the central nervous system of the subject, by way of direct administration to the bone marrow of the subject, or by way of bone marrow transplant comprising the composition.

77. The method of any one of claims 1-76, the method further comprising administering to the subject a population of cells.

78. The method of claim 77, wherein the population of cells is administered to the subject prior to administration of the composition or following administration of the composition.

79. The method of claim 77 or 78, wherein the cells are pluripotent cells or multipotent cells.

80. The method of claim 79, wherein the multipotent cells are CD34+ cells.

81. The method of claim 80, wherein the CD34+ cells are hematopoietic stem cells (HSCs) or myeloid progenitor cells (MPCs).

82. The method of claim 79, wherein the pluripotent cells are embryonic stem cells (ESCs) or induced pluripotent stem cells (iPSCs),

83. The method of any one of claims 77-79, wherein the cells are blood lineage progenitor cells (BLPCs), microglial progenitor cells, monocytes, macrophages, or microglia.

84. The method of claim 83, wherein the BLPCs are monocytes.

85. The method of any one of claims 77-84, wherein the cells are not modified to express a transgene encoding the PGRN or the GRN.

86. The method of any one of claims 1-85, wherein, prior to administration of the composition to the subject, endogenous PGRN or GRN is disrupted in the cells, in the subject, or in a population of neurons in the subject.

87. The method of claim 86, wherein the endogenous PGRN or GRN is disrupted by contacting the cells with a nuclease that catalyzes cleavage of an endogenous PGRN or GRN nucleic acid in the cells.

88. The method of claim 87, wherein the nuclease is a clustered regularly interspaced short palindromic repeats (CRISPR)-associated protein 9 (Cas9), CRISPR-associated protein is CRISPR-associated protein 12a (Cas12a), a transcription activator-like effector nuclease, a meganuclease, or a zinc finger nuclease.

89. The method of any one of claims 86-88, wherein the endogenous PGRN or GRN is disrupted by administering an inhibitory RNA molecule to the cells, the subject, or the population of neurons.

90. The method of claim 89, wherein the inhibitory RNA molecule is a short interfering RNA, a short hairpin RNA, or a miRNA.

91. The method of any one of claims 1-90, wherein the cells are autologous cells or allogeneic cells.

92. The method of any one of claims 1-91, wherein the cells are transfected or transduced ex vivo to express the PGRN or the GRN.

93. The method of claim 92, wherein the cells are transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

94. The method of claim 93, wherein the viral vector is a Retroviridae family viral vector.

95. The method of claim 94, wherein the Retroviridae family viral vector is a lentiviral vector, alpharetroviral vector, or gamma retroviral vector.

96. The method of claim 94 or 95, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

97. The method of claim 93, wherein the viral vector is an AAV selected from the group consisting of AAV1, AAV2, AAV3, AAV4, AAV5, AAV6, AAV7, AAV8, AAV9, AAV10, and AAVrh74.

98. The method of any one of claims 93-97, wherein the viral vector is a pseudotyped viral vector.

99. The method of claim 98, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

100. The method of claim 92, wherein the cells are transfected using: a) an agent selected from the group consisting of a cationic polymer, diethylaminoethyldextran, polyethylenimine, a cationic lipid, a liposome, calcium phosphate, an activated dendrimer, and a magnetic bead; or b) a technique selected from the group consisting of electroporation, Nucleofection, squeeze-poration, sonoporation, optical transfection, Magnetofection, and impalefection.

101. The method of any one of claims 1-100, wherein expression of the PGRN or the GRN in the cells is mediated by a ubiquitous promoter, a cell lineage-specific promoter, or a synthetic promoter.

102. The method of claim 101, wherein the ubiquitous promoter is selected from the group consisting of an elongation factor 1-alpha promoter and a phosphoglycerate kinase 1 promoter.

103. The method of claim 101, wherein the cell lineage-specific promoter is selected from the group consisting of a PGRN promoter, CD11 b promoter, CD68 promoter, a C-X3-C motif chemokine receptor 1 promoter, an allograft inflammatory factor 1 promoter, a purinergic receptor P2Y12 promoter, a transmembrane protein 119 promoter, and a colony stimulating factor 1 receptor promoter.

104. A pharmaceutical composition comprising a population of cells comprising a transgene encoding a PGRN or a GRN, the pharmaceutical composition further comprising one or more pharmaceutically acceptable carriers, diluent, or excipients.

105. The pharmaceutical composition of claim 104, wherein the PGRN or the GRN comprises a PGRN secretory signal peptide.

106. The pharmaceutical composition of claim 104 or 105, wherein the cells comprise a transgene encoding the PG RN.

107. The pharmaceutical composition of claim 106, wherein the PGRN comprises at least 2 GRN domains, optionally wherein the PGRN comprises at least 3 GRN domains, optionally wherein the PGRN comprises at least 4 GRN domains, optionally wherein the PGRN comprises at least 5 GRN domains, optionally wherein the PGRN comprises at least 6 GRN domains, optionally wherein the PGRN comprises at least 7 GRN domains, or optionally wherein the PGRN comprises at least 8 GRN domains.

108. The pharmaceutical composition of any one of claims 104-107, wherein the PGRN comprises from 2 to 16 GRN domains, optionally wherein the PG RN comprises from 2 to 12 GRN domains, optionally wherein the PGRN comprises from 2 to 8 GRN domains, optionally wherein the PGRN comprises from 2 to 4 GRN domains, or optionally wherein the PGRN comprises 2 GRN domains.

109. The pharmaceutical composition of any one of claims 104-108, wherein the PGRN comprises a para-GRN domain having an amino acid sequence that is at least 85% identical to the amino acid sequence of SEQ ID NO. 2, optionally wherein the para-GRN domain has an amino acid sequence that is at least 90% identical to the amino acid sequence of SEQ ID NO. 2, optionally wherein the para-GRN domain has an amino acid sequence that is at least 95% identical to the amino acid sequence of SEQ ID NO. 2, or optionally wherein the para-GRN domain has an amino acid sequence of SEQ ID NO. 2.

110. The pharmaceutical composition of any one of claims 104-109, wherein the PGRN comprises a GRN-1 domain having an amino acid sequence that is at least 85% identical to the amino acid sequence of SEQ ID NO. 3, optionally wherein the GRN-1 domain has an amino acid sequence that is at least 90% identical to the amino acid sequence of SEQ ID NO. 3, optionally wherein the GRN-1 domain has an amino acid sequence that is at least 95% identical to the amino acid sequence of SEQ ID NO. 3, or optionally wherein the GRN-1 domain has the amino acid sequence of SEQ ID NO. 3.

111. The pharmaceutical composition of any one of claims 104-110, wherein the PGRN comprises a GRN-2 domain having an amino acid sequence that is at least 85% identical to the amino acid sequence of SEQ ID NO. 4, optionally wherein the GRN-2 domain has an amino acid sequence that is at least 90% identical to the amino acid sequence of SEQ ID NO. 4, optionally wherein the GRN-2 domain has an amino acid sequence that is at least 95% identical to the amino acid sequence of SEQ ID NO. 4, or optionally wherein the GRN-2 domain has an amino acid sequence of SEQ ID NO. 4.

112. The pharmaceutical composition of any one of claims 104-111, wherein the PGRN comprises a GRN-3 domain having an amino acid sequence that is at least 85% identical to the amino acid sequence of SEQ ID NO. 5, optionally wherein the GRN-3 domain has an amino acid sequence that is at least 90% identical to the amino acid sequence of SEQ ID NO. 5, optionally wherein the GRN-3 domain has an amino acid sequence that is at least 95% identical to the amino acid sequence of SEQ ID NO. 5, or optionally wherein the GRN-3 domain has an amino acid sequence of SEQ ID NO. 5.

113. The pharmaceutical composition of any one of claims 104-112, wherein the PGRN comprises a GRN-4 domain having an amino acid sequence that is at least 85% identical to the amino acid sequence of SEQ ID NO. 6, optionally wherein the GRN-4 domain has an amino acid sequence that is at least 90% identical to the amino acid sequence of SEQ ID NO. 6, optionally wherein the GRN-4 domain has an amino acid sequence that is at least 95% identical to the amino acid sequence of SEQ ID NO. 6, or optionally wherein the GRN-4 domain has an amino acid sequence of SEQ ID NO. 6.

114. The pharmaceutical composition of any one of claims 104-113, wherein the PGRN comprises a GRN-5 domain having an amino acid sequence that is at least 85% identical to the amino acid sequence of SEQ ID NO. 7, optionally wherein the GRN-5 domain has an amino acid sequence that is at least 90% identical to the amino acid sequence of SEQ ID NO. 7, optionally wherein the GRN-5 domain has an amino acid sequence that is at least 95% identical to the amino acid sequence of SEQ ID NO. 7, or optionally wherein the GRN-5 domain has an amino acid sequence of SEQ ID NO. 7.

115. The pharmaceutical composition of any one of claims 104-114, wherein the PGRN comprises a GRN-6 domain having an amino acid sequence that is at least 85% identical to the amino acid sequence of SEQ ID NO. 8, optionally wherein the GRN-6 domain has an amino acid sequence that is at least 90% identical to the amino acid sequence of SEQ ID NO. 8, optionally wherein the GRN-6 domain has an amino acid sequence that is at least 95% identical to the amino acid sequence of SEQ ID NO. 8, or optionally wherein the GRN-6 domain has an amino acid sequence of SEQ ID NO. 8.

116. The pharmaceutical composition of any one of claims 104-115, wherein the PGRN comprises a GRN-7 domain having an amino acid sequence that is at least 85% identical to the amino acid sequence of SEQ ID NO. 9, optionally wherein the GRN-7 domain has an amino acid sequence that is at least 90% identical to the amino acid sequence of SEQ ID NO. 9, optionally wherein the GRN-7 domain has an amino acid sequence that is at least 95% identical to the amino acid sequence of SEQ ID NO. 9, or optionally wherein the GRN-7 domain has an amino acid sequence of SEQ ID NO. 9.

117. The pharmaceutical composition of any one of claims 104-116, wherein the PGRN is a full-length PGRN.

118. The pharmaceutical composition of claim 117, wherein the PGRN has an amino acid sequence that is at least 85% identical to the amino acid sequence of SEQ ID NO. 1, optionally wherein the PGRN has an amino acid sequence that is at least 90% identical to the amino acid sequence of SEQ ID NO. 1, optionally wherein the PGRN has an amino acid sequence that is at least 95% identical to the amino acid sequence of SEQ ID NO. 1, optionally wherein the PG RN has an amino acid sequence of SEQ ID NO. 1, or optionally wherein the cells comprise a transgene encoding the GRN.

119. The pharmaceutical composition of any one of claims 104-118, wherein the GRN is a para-GRN domain having an amino acid sequence that is at least 85% identical to the amino acid sequence of SEQ ID NO. 2, optionally wherein the para-GRN domain has an amino acid sequence that is at least 90% identical to the amino acid sequence of SEQ ID NO. 2, optionally wherein the para-GRN domain has an amino acid sequence that is at least 95% identical to the amino acid sequence of SEQ ID NO. 2, or optionally wherein the para-GRN domain has an amino acid sequence of SEQ ID NO. 2.

120. The pharmaceutical composition of any one of claims 104-119, wherein the GRN is a GRN-1 domain having an amino acid sequence that is at least 85% identical to the amino acid sequence of SEQ ID NO. 3, optionally wherein the GRN-1 domain has an amino acid sequence that is at least 90% identical to the amino acid sequence of SEQ ID NO. 3, optionally wherein the GRN-1 domain has an amino acid sequence that is at least 95% identical to the amino acid sequence of SEQ ID NO. 3, or optionally wherein the GRN-1 domain has an amino acid sequence of SEQ ID NO. 3.

121. The pharmaceutical composition of any one of claims 104-120, wherein the GRN is a GRN-2 domain having an amino acid sequence that is at least 85% identical to the amino acid sequence of SEQ ID NO. 4, optionally wherein the GRN-2 domain has an amino acid sequence that is at least 90% identical to the amino acid sequence of SEQ ID NO. 4, optionally wherein the GRN-2 domain has an amino acid sequence that is at least 95% identical to the amino acid sequence of SEQ ID NO. 4, or optionally wherein the GRN-2 domain has an amino acid sequence of SEQ ID NO. 4.

122. The pharmaceutical composition of any one of claims 104-121, wherein the GRN is a GRN-3 domain having an amino acid sequence that is at least 85% identical to the amino acid sequence of SEQ ID NO. 5, optionally wherein the GRN-3 domain has an amino acid sequence that is at least 90% identical to the amino acid sequence of SEQ ID NO. 5, optionally wherein the GRN-3 domain has an amino acid sequence that is at least 95% identical to the amino acid sequence of SEQ ID NO. 5, or optionally wherein the GRN-3 domain has an amino acid sequence of SEQ ID NO. 5.

123. The pharmaceutical composition of any one of claims 104-122, wherein the GRN-4 domain has an amino acid sequence that is at least 85% identical to the amino acid sequence of SEQ ID NO. 6, optionally wherein the GRN-4 domain has an amino acid sequence that is at least 90% identical to the amino acid sequence of SEQ ID NO. 6, optionally wherein the GRN-4 domain has an amino acid sequence that is at least 95% identical to the amino acid sequence of SEQ ID NO. 6, optionally wherein the GRN-4 domain has an amino acid sequence of SEQ ID NO. 6.

124. The pharmaceutical composition of any one of claims 104-123, wherein the GRN is a GRN-5 domain having an amino acid sequence that is at least 85% identical to the amino acid sequence of SEQ ID NO. 7, optionally wherein the GRN-5 domain has an amino acid sequence that is at least 90% identical to the amino acid sequence of SEQ ID NO. 7, optionally wherein the GRN-5 domain has an amino acid sequence that is at least 95% identical to the amino acid sequence of SEQ ID NO. 7, or optionally wherein the GRN-5 domain has an amino acid sequence of SEQ ID NO. 7.

125. The pharmaceutical composition of any one of claims 104-124, wherein the GRN is a GRN-6 domain having an amino acid sequence that is at least 85% identical to the amino acid sequence of SEQ ID NO. 8, optionally wherein the GRN-6 domain has an amino acid sequence that is at least 90% identical to the amino acid sequence of SEQ ID NO. 8, optionally wherein the GRN-6 domain has an amino acid sequence that is at least 95% identical to the amino acid sequence of SEQ ID NO. 8, or optionally wherein the GRN-6 domain has an amino acid sequence of SEQ ID NO. 8.

126. The pharmaceutical composition of any one of claims 104-125, wherein the GRN is a GRN-7 domain having an amino acid sequence that is at least 85% identical to the amino acid sequence of SEQ ID NO. 9, optionally wherein the GRN-7 domain has an amino acid sequence that is at least 90% identical to the amino acid sequence of SEQ ID NO. 9, optionally wherein the GRN-7 domain has an amino acid sequence that is at least 95% identical to the amino acid sequence of SEQ ID NO. 9, or optionally wherein the GRN-7 domain has an amino acid sequence of SEQ ID NO. 9.

127. The pharmaceutical composition of any one of claims 104-126, wherein the GRN is a full-length GRN.

128. The pharmaceutical composition of any one of claims 104-126, wherein the cells comprise a PGRN transgene having at least 85% sequence identity to the nucleic acid sequence of SEQ ID NO. 10, optionally wherein the PGRN transgene has at least 90% sequence identity to the nucleic acid sequence of SEQ ID NO. 10, optionally wherein the PGRN transgene has at least 95% sequence identity to the nucleic acid sequence of SEQ ID NO. 10, or optionally wherein the PGRN transgene has the nucleic acid sequence of SEQ ID NO. 10.

129. The pharmaceutical composition of any one of claims 104-128, wherein the cells comprise a codon-optimized PGRN transgene.

130. The pharmaceutical composition of claim 129, wherein the codon-optimized transgene has at least 85% sequence identity to the nucleic acid sequence of SEQ ID NO. 19, optionally, wherein the codon-optimized transgene has at least 90% sequence identity to the nucleic acid sequence of SEQ ID NO. 19, optionally, wherein the codon-optimized transgene has at least 95% sequence identity to the nucleic acid sequence of SEQ ID NO. 19, or optionally, wherein the codon-optimized transgene has the nucleic acid sequence of SEQ ID NO. 19.

131. The pharmaceutical composition of any one of claims 104-130, wherein the PGRN or the GRN is a PGRN or a GRN fusion protein.

132. The pharmaceutical composition of claim 131, wherein the PGRN or the GRN fusion protein comprises a Rb domain of ApoE.

133. The pharmaceutical composition of claim 132, wherein the Rb domain comprises a portion of ApoE having the amino acid sequence of residues 25-185, 50-180, 75-175, 100-170, 125-160, or 130-150 of SEQ ID NO. 11.

134. The pharmaceutical composition of claim 132 or 133, wherein the Rb domain comprises a region having at least 70% sequence identity to the amino acid sequence of residues 159-167 of SEQ ID NO. 11.

135. The pharmaceutical composition of claim 131, wherein the PGRN OR GRN fusion protein comprises PGRN OR GRN and a glycosylation independent lysosomal targeting (GILT) tag.

136. The pharmaceutical composition of claim 135, wherein the GILT tag comprises a human IGF-II mutein having an amino acid sequence that is at least 70% identical to the amino acid sequence of mature human IGF-II (SEQ ID NO. 12), and having diminished binding affinity for the insulin receptor relative to the affinity of naturally-occurring human IGF-II for the insulin receptor, wherein the IGF-II mutein is resistant to furin cleavage and binds to the human cation-independent mannose-6-phosphate receptor in a mannose-6-phosphate-independent manner.

137. The pharmaceutical composition of claim 136, wherein the IGF-II mutein comprises a mutation within a region corresponding to amino acids 30-40 of SEQ ID NO. 12, and wherein the mutation abolishes at least one furin protease cleavage site.

138. The pharmaceutical composition of claim 137, wherein the mutation is an amino acid substitution, deletion, and/or insertion.

139. The pharmaceutical composition of claim 138, wherein the mutation is a Lys or Ala amino acid substitution at a position corresponding to Arg37 or Arg40 of SEQ ID NO. 12.

140. The pharmaceutical composition of claim 138, wherein the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO. 12, and combinations thereof.

141. The pharmaceutical composition of any one of claims 135-140, wherein the GILT tag has an amino acid sequence that is at least 70% identical to the amino acid sequence of SEQ ID NO. 13, optionally wherein the GILT tag has an amino acid sequence that is at least 80% identical to the amino acid sequence of SEQ ID NO. 13, optionally wherein the GILT tag has an amino acid sequence that is at least 90% identical to the amino acid sequence of SEQ ID NO. 13, optionally wherein the GILT tag has the amino acid sequence of SEQ ID NO. 13.

142. The pharmaceutical composition of any one of claims 135-140, wherein the GILT tag has an amino acid sequence that is at least 70% identical to the amino acid sequence of SEQ ID NO. 14, optionally wherein the GILT tag has an amino acid sequence that is at least 80% identical to the amino acid sequence of SEQ ID NO. 14, optionally wherein the GILT tag has an amino acid sequence that is at least 90% identical to the amino acid sequence of SEQ ID NO. 14, optionally wherein the GILT tag has the amino acid sequence of SEQ ID NO. 14.

143. The pharmaceutical composition of any one of claims 135-140, wherein the GILT tag has an amino acid sequence that is at least 70% identical to the amino acid sequence of SEQ ID NO. 15, optionally wherein the GILT tag has an amino acid sequence that is at least 80% identical to the amino acid sequence of SEQ ID NO. 15, optionally wherein the GILT tag has an amino acid sequence that is at least 90% identical to the amino acid sequence of SEQ ID NO. 15, optionally wherein the GILT tag has the amino acid sequence of SEQ ID NO. 15.

144. The pharmaceutical composition of any one of claims 135-140, wherein the GILT tag is encoded by a polynucleotide having a nucleic acid sequence that is at least 85% identical to the nucleic acid sequence of SEQ ID NO. 16, optionally wherein the GILT tag is encoded by a polynucleotide having a nucleic acid sequence that is at least 90% identical to the nucleic acid sequence of SEQ ID NO. 16, optionally wherein the GILT tag is encoded by a polynucleotide having a nucleic acid sequence that is at least 95% identical to the nucleic acid sequence of SEQ ID NO. 16, optionally wherein the GILT tag is encoded by a polynucleotide having the nucleic acid sequence of SEQ ID NO. 16.

145. The pharmaceutical composition of any one of claims 135-140, wherein the GILT tag is encoded by a polynucleotide having a nucleic acid sequence that is at least 85% identical to the nucleic acid sequence of SEQ ID NO. 17, optionally wherein the GILT tag is encoded by a polynucleotide having a nucleic acid sequence that is at least 90% identical to the nucleic acid sequence of SEQ ID NO. 17, optionally wherein the GILT tag is encoded by a polynucleotide having a nucleic acid sequence that is at least 95% identical to the nucleic acid sequence of SEQ ID NO. 17, optionally wherein the GILT tag is encoded by a polynucleotide having the nucleic acid sequence of SEQ ID NO. 17.

146. The pharmaceutical composition of any one of claims 135-140, wherein the GILT tag is encoded by a polynucleotide having a nucleic acid sequence that is at least 85% identical to the nucleic acid sequence of SEQ ID NO. 18, optionally wherein the GILT tag is encoded by a polynucleotide having a nucleic acid sequence that is at least 90% identical to the nucleic acid sequence of SEQ ID NO. 18, optionally wherein the GILT tag is encoded by a polynucleotide having a nucleic acid sequence that is at least 95% identical to the nucleic acid sequence of SEQ ID NO. 18, optionally wherein the GILT tag is encoded by a polynucleotide having the nucleic acid sequence of SEQ ID NO. 18.

147. The pharmaceutical composition of any one of claims 104-146, wherein the transgene encoding PGRN or GRN further comprises a miR-126 targeting sequence in the 3′-UTR.

148. The composition of any one of claims 104-147, wherein the cells are pluripotent cells or multipotent cells.

149. The composition of claim 148, wherein the multipotent cells are CD34+ cells.

150. The composition of claim 149, wherein the CD34+ cells are HSCs or MPCs.

151. The composition of claim 148, wherein the pluripotent cells are ESCs or iPSCs.

152. The composition of any one of claims 104-147, wherein the cells are BLPCs, microglial progenitor cells, macrophages, or microglia.

153. The composition of claim 152, wherein the BLPCs are monocytes.

154. The pharmaceutical composition of any one of claims 104-153, wherein the cells are transfected or transduced ex vivo to express the PGRN or the GRN.

155. A kit comprising the pharmaceutical composition of any one of claims 104-154 and a package insert.

156. The kit of claim 155, wherein the package insert instructs a user of the kit to perform the method of any one of claims 1-103.
